(19) United States
(12) Patent Application Publication
(10) Pub. No.: US 2022/0193205 A1
(43) Pub. Date: Jun. 23, 2022

(54) METHODS OF TREATING CANCER

(71) Applicant: Foghorn Therapeutics Inc., Cambridge, MA (US)

(72) Inventors: Qianhe ZHOU, Winchester, MA (US); Ho Man CHAN, Carlisle, MA (US); Luis SOARES, Cambridge, MA (US); Cong ZHU, Newton, MA (US)

(21) Appl. No.: 17/420,296

(22) PCT Filed: Jan. 7, 2020
(86) PCT No.: PCT/US2020/012615

§ 371 Date: Jul. 1, 2021

Related U.S. Application Data
(60) Provisional application No. 62/789,346, filed on Jan. 7, 2019.

(51) Int. Cl. A61K 38/46; A61K 31/7105; A61K 31/704; A61K 33/243; A61K 31/675; A61K 31/519; A61K 31/166; A61K 31/175; A61K 31/475; A61K 31/495; A61K 38/12; A61P 35/00

(52) U.S. Cl. CPC A61K 38/465; A61K 31/7105; A61K 31/704; A61K 33/243; A61K 31/675; A61P 35/00; A61K 31/166; A61K 31/175; A61K 31/475; A61K 31/495; A61K 38/12; A61K 31/519

(57) ABSTRACT

The present disclosure features useful methods to treat an ALT-positive cancer and/or a cancer having a deficiency in ATRX and/or DAXX, e.g., in a subject in need thereof. In some embodiments, the methods described herein are useful in the treatment of cancer in combination with anti-cancer therapies.

### SEQUENCE LISTING

The instant application contains a Sequence Listing which has been submitted electronically in ASCII format and is hereby incorporated by reference in its entirety. Said ASCII copy, created on Mar. 4, 2022 is named 51121-015002_Sequence_Listing_2_22_22_ST25 and is 111,691 bytes in size.

### BACKGROUND

Cancer remains one of the deadliest threats to human health and is the second leading cause of mortality. In 2012, there were an estimated 14.1 million cases of cancer diagnosed around the world and 8.2 million cancer deaths. By 2030, the global burden is expected to reach 21.6 million new cancer cases and 13.0 million cancer deaths annually. Thus, there is a need to develop new approaches for the treatment of cancer.

### SUMMARY OF THE INVENTION

The present invention features methods to treat alternative lengthening of telomeres (ALT)-positive cancer. The present invention also features methods to treat cancer having mutations in the ATRX and/or DAXX genes, e.g., in a subject in need thereof.

In one aspect, the invention features a method of treating an ALT-positive cancer in a subject in need thereof. This method includes administering to the subject an effective amount of an agent that reduces the level and/or activity of SWI/SNF-related matrix-associated actin-dependent regulator of chromatin subfamily A-like protein 1 (SMARCAL1) in a cell in the subject.

In another aspect, the invention features a method of reducing the level and/or activity of SMARCAL1 in an ALT-positive cancer cell in a subject. This method includes contacting the cell with an effective amount of an agent that reduces the level and/or activity of SMARCAL1 in the cell.

In another aspect, the invention features a method of reducing tumor growth of an ALT-positive-cancer in a subject. This method includes administering to the subject an effective amount of an agent that reduces the level and/or activity of SMARCAL1 in a cell in the subject.

In some embodiments of any of the above aspects, the ALT-positive-cancer is associated with a mutation in the ATRX gene. In some embodiments, the mutation in the ATRX gene is a mutation that results in a loss of function of ATRX. In some embodiments, the ALT-positive cancer is associated with a mutation in the DAXX gene. In some embodiments, the mutation in the DAXX gene is a mutation that results in a loss of function of DAXX.

In another aspect, the invention features a method of treating a cancer having a mutation that results in a loss of function of ATRX and/or DAXX in a subject in need thereof. This method includes administering to the subject an effective amount of an agent that reduces the level and/or activity of SMARCAL1 in the subject.

In another aspect, the invention features a method of reducing the level and/or activity of SMARCAL1 in a cancer cell having a mutation that results in a loss of function of ATRX and/or DAXX in a subject. This method includes contacting the cell with an effective amount of an agent that reduces the level and/or activity of SMARCAL1 in the cell.

In another aspect, the invention features a method of reducing tumor growth of a cancer having a mutation that results in a loss of function of ATRX and/or DAXX in a subject. This method includes administering to a subject an effective amount of an agent that reduces the level and/or activity of SMARCAL1 in a cell in the subject.

In some embodiments of any of the above aspects, the agent that reduces the level and/or activity of SMARCAL1 is a nuclease. In some embodiments of any of the above aspects, the agent that reduces the level and/or activity of SMARCAL1 is a polynucleotide. In some embodiments of any of the above aspects, the agent that reduces the level and/or activity of SMARCAL1 is a small-molecule compound. In some embodiments of any of the above aspects, the agent that reduces the level and/or activity of SMARCAL1 is an antibody. In some embodiments of any of the above aspects, the agent that reduces the level and/or activity of SMARCAL1 is an enzyme.

In some embodiments, the agent that reduces the level and/or activity of SMARCAL1 is a nuclease. In some embodiments, the nuclease is a clustered regularly interspaced short palindromic repeats (CRISPR)-associated protein. In some embodiments, the CRISPR-associated protein is CRISPR-associated protein 9 (Cas9). In some embodiments, the nuclease is a transcription activator-like effector nuclease (TALEN). In some embodiments, the nuclease is a meganuclease. In some embodiments, the nuclease is a zinc finger nuclease (ZFN).

In some embodiments, the agent that reduces the level and/or activity of SMARCAL1 is a polynucleotide. In some embodiments, the polynucleotide is an antisense nucleic acid. In some embodiments, the polynucleotide is a CRISPR/Cas 9 nucleotide. In some embodiments, the polynucleotide is a short interfering RNA (siRNA). In some embodiments, the polynucleotide is a short hairpin RNA (shRNA). In some embodiments, the polynucleotide is a micro RNA (miRNA). In some embodiments, the polynucleotide is a ribozyme. In some embodiments, the polynucleotide comprises a sequence having at least 85% sequence identity (e.g., 85%, 86%, 87%, 88%, 89%, 90%, 91%, 92%, 93%, 94%, 95%, 96%, 97%, 98%, 99%, 99.9% identity, or more) to the nucleic acid sequence of any one of SEQ ID NOs: 7-45. In other embodiments, the polynucleotide comprises a sequence having at least 85% sequence identity (e.g., 85%, 86%, 87%, 88%, 89%, 90%, 91%, 92%, 93%, 94%, 95%, 96%, 97%, 98%, 99%, 99.9% identity, or more) to the nucleic acid sequence of any one of SEQ ID NOs: 7-9.

In some embodiments of any of the above aspects, the method further comprises administering to the subject an anti-cancer therapy. In some embodiments, the anti-cancer therapy is a telomerase inhibitor. In some embodiments, the telomerase inhibitor is a vaccine (e.g., a peptide vaccine such as RIAVAX™ (tertomotide)) that activates an immune response against telomerase. In other embodiments, the telomerase inhibitor binds to and inhibits telomerase activity (e.g., the oligonucleotide-lipid conjugate imetelstat). In some embodiments, the anti-cancer therapy is a small molecule that induces DNA damage and/or modulates a DNA-repair pathway and/or a replication stress pathway. Such anti-cancer therapies include, for example, calactin, PARP inhibitors (e.g., olaparib, niraparib, rucaparib, veliparib, CEP9722 (i.e., 11-methoxy-2-((4-methylpiperazin-1-yl(methyl)-4,5,6,7-tetrahydro-1H-cyclopenta[a]pyrrolo[3,4-c]carbazole-1,3(2H)-dione), E7016 (i.e., 10-((4-Hydroxypiperidin-1-yl(methyl)chromeno[4,3,2-de]phthalazin-3(2H)-one), iniparib, talazoparib, pamiparib, or 3-aminobenzamide), CHK1/2 inhibitors (e.g., prexasertib, UCN-01, CHIR-124, AZD7762, PF477736, PD-321852, SAR-020106, CCT244747, SCH900776, LY2603618, V158411, NSC109555, PV1019, VRX0466617, or CCT241533), DNA-PKCS inhibitors (e.g., NU7441 (also known as KU-57788), AZD7648, PI-103, PIK-75 HCI, NU7026, PP121, KU-0060648, CC-115, SF2523, samotolisib, YU238259, or LTURM34), ATR inhibitor (AZD6738, schisandrin B, ETP-46464, NU6027, VE-821, VE-822, or AZ20) and CDK4/6 inhibitors (e.g., palbociclib, arcyriaflavin, NSC625987, PD 0332991 isethionate, ribociclib, ryuvidine, BSJ-03-123, BSJ-03-204, or BSJ-04-132). In some embodiments, e.g., if the cancer is an osteosarcoma, the anti-cancer therapy is doxorubicin, cisplatin, ifosfamide, or high-dose methotrexate (MTX) with leucovorin rescue, or combinations thereof. In some embodiments, e.g., if the cancer is an astrocytoma such as a diffuse of infiltrating astrocytoma, the anti-cancer therapy is surgery, temozolomide, radiation therapy, procarbazine, lomustine, or vincristine, or combinations thereof. In some embodiments, e.g., if the cancer is an astrocytoma such as an anaplastic astrocytoma, the anti-cancer therapy is surgery, radiation therapy, temozolomide, carmustine, lomustine, cisplatin, procarbazine, or vincristine, or combinations thereof. In some embodiments, e.g., if the cancer is a rhabdomyosarcoma, the anti-cancer therapy is vincristine, dactinomycin, or cyclophosphamide, or combinations thereof.

In some embodiments, the agent that reduces the level and/or of SMARCAL1 is administered systemically to the subject. In some embodiments, the agent that reduces the level and/or of SMARCAL1 is administered intratumorally to the subject.

In some embodiments of any of the above aspects, the subject has a cancer that is refractory to an anti-cancer therapy. In some embodiments, the anti-cancer therapy is a telomerase inhibitor.

In some embodiments, the cancer is a soft tissue sarcoma. In some embodiments, the cancer is an osteosarcoma. In some embodiments, the cancer is a rhabdomyosarcoma. In some embodiments, the cancer is a pancreatic neuroendocrine tumor (PanNET). In some embodiments, the cancer is a glioma. In some embodiments, the cancer is a glioblastoma. In some embodiments, the cancer is a pediatric glioblastoma. In some embodiments, the cancer is an astrocytoma. In some embodiments, the cancer is an endometrial cancer. In some embodiments, the cancer is an adrenocortical carcinoma. In some embodiments, the cancer is a neuroepithelial tumor. In some embodiments, the cancer is a non-small cell lung cancer. In some embodiments, the cancer is a bladder cancer. In some embodiments, the cancer is an esophagogastric cancer. In some embodiments, the cancer is a melanoma. In some embodiments, the cancer is a head and neck cancer. In some embodiments, the cancer is a cervical cancer. In some embodiments, the cancer is a Non-Hodgkin lymphoma. In some embodiments, the cancer is a colorectal cancer. In some embodiments, the cancer is a pancreatic cancer. In some embodiments, the cancer is a germ cell tumor. In some embodiments, the cancer is a breast cancer. In some embodiments, the cancer is an ovarian cancer. In some embodiments, the cancer is a hepatobiliary cancer. In some embodiments, the cancer is a renal cell carcinoma. In some embodiments, the cancer is a pheochromocytoma. In some embodiments, the cancer is a prostate cancer. In some embodiments, the cancer is a thyroid cancer. In some embodiments, the cancer is an adrenal gland/peripheral nervous system cancer. In some embodiments, the cancer is a central nervous system cancer. In some embodiments, the cancer is a gall bladder cancer. In some embodiments, the cancer is a hematopoietic neoplasm. In some embodiments, the cancer is a larynx cancer. In some embodiments, the cancer is a liver cancer. In some embodiments, the cancer is an oral cavity cancer. In some embodiments, the cancer is a pleural cancer. In some embodiments, the cancer is a salivary gland carcinoma. In some embodiments, the cancer is a skin cancer. In some embodiments, the cancer is a small intestine cancer. In some embodiments, the cancer is a stomach cancer. In some embodiments, the cancer is a tendon sheath cancer. In some embodiments, the cancer is a testicular cancer. In some embodiments, the cancer is a uterine cancer. In some embodiments, the cancer is a ganglioneuroblastoma. In some embodiments, the cancer is a diffuse astrocytoma. In some embodiments, the cancer is an anaplastic astrocytoma. In some embodiments, the cancer is a glioblastoma multiforme. In some embodiments, the cancer is an oligodendroglioma. In some embodiments, the cancer is an anaplastic medulloblastoma. In some embodiments, the cancer is a paraganglioma. In some embodiments, the cancer is an undifferentiated pleomorphic sarcoma. In some embodiments, the cancer is a fibrosarcoma. In some embodiments, the cancer is a leiomyosarcoma. In some embodiments, the cancer is a liposarcoma. In some embodiments, the cancer is an angiosargoma. In some embodiments, the cancer is an epithelioid sarcoma. In some embodiments, the cancer is a nonseminoumatous germ cell tumor.

In some embodiments of any of the above aspects, the subject is a human.

In another aspect, the invention features a kit including a pharmaceutical composition including an agent that reduces the level and/or activity of SMARCAL1 in a cell in a subject and a package insert with instructions to perform any of the methods described herein. In some embodiments, the kit additionally includes an additional therapeutic agent (e.g., an anti-cancer agent, e.g., a telomerase inhibitor).

In another aspect, the invention features a method of reducing growth of an ALT-positive cancer cell. This method includes contacting the cell with an effective amount of an agent that reduces the level and/or activity of SMARCAL1 in the cell.

In another aspect, the invention features a method of reducing growth of cancer cell having a mutation that results in a loss of function of ATRX and/or DAXX. This method includes contacting the cell with an effective amount of an agent that reduces the level and/or activity of SMARCAL1 in the cell.

In some embodiments of either of the above aspects, the cancer cell is a soft tissue sarcoma cell. In some embodiments, the cancer cell is an osteosarcoma cell. In some embodiments, the cancer cell is a rhabdomyosarcoma cell. In some embodiments, the cancer cell is a PanNET cell. In some embodiments, the cancer cell is a glioma cell. In some embodiments, the cancer cell is a glioblastoma cell. In some embodiments, the cancer cell is a pediatric glioblastoma cell. In some embodiments, the cancer cell is an astrocytoma cell. In some embodiments, the cancer cell is an endometrial cancer cell. In some embodiments, the cancer cell is an adrenocortical carcinoma cell. In some embodiments, the cancer cell is a neuroepithelial tumor cell. In some embodiments, the cancer cell is a non-small cell lung cancer cell. In some embodiments, the cancer cell is a bladder cancer cell. In some embodiments, the cancer cell is an esophagogastric cancer cell. In some embodiments, the cancer cell is a melanoma cell. In some embodiments, the cancer cell is a head and neck cancer cell. In some embodiments, the cancer cell is a cervical cancer cell. In some embodiments, the cancer cell is a Non-Hodgkin lymphoma cell. In some embodiments, the cancer cell is a colorectal cancer cell. In some embodiments, the cancer cell is a pancreatic cancer cell. In some embodiments, the cancer cell is a germ cell tumor cell. In some embodiments, the cancer cell is a breast cancer cell. In some embodiments, the cancer cell is an ovarian cancer cell. In some embodiments, the cancer cell is a hepatobiliary cancer cell. In some embodiments, the cancer cell is a renal cell carcinoma cell. In some embodiments, the cancer cell is a pheochromocytoma cell. In some embodiments, the cancer cell is a prostate cancer cell. In some embodiments, the cancer cell is a thyroid cancer cell. In some embodiments, the cancer cell is an adrenal gland/peripheral nervous system cancer cell. In some embodiments, the cancer cell is a central nervous system cancer cell. In some embodiments, the cancer cell is a gall bladder cancer cell. In some embodiments, the cancer cell is a hematopoietic neoplasm cell. In some embodiments, the cancer cell is a larynx cancer cell. In some embodiments, the cancer cell is a liver cancer cell. In some embodiments, the cancer cell is an oral cavity cancer cell. In some embodiments, the cancer cell is a pleural cancer cell. In some embodiments, the cancer cell is a salivary gland carcinoma cell. In some embodiments, the cancer cell is a skin cancer cell. In some embodiments, the cancer cell is a small intestine cancer cell. In some embodiments, the cancer cell is a stomach cancer cell. In some embodiments, the cancer cell is a tendon sheath cancer cell. In some embodiments, the cancer cell is a testicular cancer cell. In some embodiments, the cancer cell is a uterine cancer cell. In some embodiments, the cancer cell is a ganglioneuroblastoma cell. In some embodiments, the cancer cell is a diffuse astrocytoma cell. In some embodiments, the cancer cell is an anaplastic astrocytoma cell. In some embodiments, the cancer cell is a glioblastoma multiforme cell. In some embodiments, the cancer cell is an oligodendroglioma cell. In some embodiments, the cancer cell is an anaplastic medulloblastoma cell. In some embodiments, the cancer cell is a paraganglioma cell. In some embodiments, the cancer cell is an undifferentiated pleomorphic sarcoma cell. In some embodiments, the cancer cell is a fibrosarcoma cell. In some embodiments, the cancer cell is a leiomyosarcoma cell. In some embodiments, the cancer cell is a liposarcoma cell. In some embodiments, the cancer cell is an angiosargoma cell. In some embodiments, the cancer cell is an epithelioid sarcoma cell. In some embodiments, the cancer cell is a nonseminoumatous germ cell tumor cell. In any of these embodiments, the cancer cell may be a human cancer cell.

Definitions

In this application, unless otherwise clear from context, (i) the term “a” may be understood to mean “at least one”; (ii) the term “or” may be understood to mean “and/or”; and (iii) the terms “including” and “includes” may be understood to encompass itemized components or steps whether presented by themselves or together with one or more additional components or steps.

As used herein, the terms “about” and “approximately” refer to a value that is within 10% above or below the value being described. For example, the term “about 5 nM” indicates a range of from 4.5 to 5.5 nM.

As used herein, the term “administration” refers to the administration of a composition (e.g., a compound or a preparation that includes a therapeutic agent as described herein e.g., an anti-SMARCAL1 agent) to a subject or system. Administration to an animal subject (e.g., to a human) may be by any appropriate route. For example, in some embodiments, administration may be systemic (including intravenous), intratumoral, bronchial, buccal, enteral, interdermal, intra-arterial, intradermal, intragastric, intramedullary, intramuscular, intranasal, intraperitoneal, intrathecal, intraventricular, mucosal, nasal, oral, rectal, subcutaneous, sublingual, topical, tracheal, transdermal, vaginal, or vitreal.

The term “cancer” refers to a condition caused by the proliferation of malignant neoplastic cells, such as tumors, neoplasms, carcinomas, sarcomas, leukemias, and lymphomas.

As used herein, a “combination therapy” and “administered in combination” mean that two (or more) different agents or treatments are administered to a subject as part of a defined treatment regimen for a particular disease or condition. The treatment regimen defines the doses and periodicity of administration of each agent such that the effects of the separate agents on the subject overlap. In some embodiments, the delivery of the two or more agents is simultaneous or concurrent and the agents may be co-formulated. In some embodiments, the two or more agents are not co-formulated and are administered in a sequential manner as part of a prescribed regimen. In some embodiments, administration of two or more agents or treatments in combination is such that the reduction in a symptom, or other parameter related to the disorder is greater than what would be observed with one agent or treatment delivered alone or in the absence of the other. The effect of the two treatments can be partially additive, wholly additive, or greater than additive (e.g., synergistic). Sequential or substantially simultaneous administration of each therapeutic agent can be effected by any appropriate route including, but not limited to, oral routes, intravenous routes, intramuscular routes, and direct absorption through mucous membrane tissues. The therapeutic agents can be administered by the same route or by different routes. For example, a first therapeutic agent of the combination may be administered by intravenous injection while a second therapeutic agent of the combination may be administered orally.

By “determining the level of a protein” is meant the detection of a protein, or an mRNA encoding the protein, by methods known in the art either directly or indirectly. “Directly determining” means performing a process (e.g., performing an assay or test on a sample or “analyzing a sample” as that term is defined herein) to obtain the physical entity or value. “Indirectly determining” refers to receiving the physical entity or value from another party or source (e.g., a third-party laboratory that directly acquired the physical entity or value). Methods to measure protein level generally include, but are not limited to, western blotting, immunoblotting, enzyme-linked immunosorbent assay (ELISA), radioimmunoassay (RIA), immunoprecipitation, immunofluorescence, surface plasmon resonance, chemiluminescence, fluorescent polarization, phosphorescence, immunohistochemical analysis, matrix-assisted laser desorption/ionization time-of-flight (MALDI-TOF) mass spectrometry, liquid chromatography (LC)-mass spectrometry, microcytometry, microscopy, fluorescence activated cell sorting (FACS), and flow cytometry, as well as assays based on a property of a protein including, but not limited to, enzymatic activity or interaction with other protein partners. Methods to measure mRNA levels are known in the art. By “level” is meant a level or activity of a protein, or mRNA encoding the protein, as compared to a reference. The reference can be any useful reference, as defined herein. By a “decreased level” or an “increased level” of a protein is meant a decrease or increase in protein level, as compared to a reference (e.g., a decrease or an increase by about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 100%, about 150%, about 200%, about 300%, about 400%, about 500%, or more; a decrease or an increase of more than about 10%, about 15%, about 20%, about 50%, about 75%, about 100%, or about 200%, as compared to a reference; a decrease or an increase by less than about 0.01-fold, about 0.02-fold, about 0.1-fold, about 0.3-fold, about 0.5-fold, about 0.8-fold, or less; or an increase by more than about 1.2-fold, about 1.4-fold, about 1.5-fold, about 1.8-fold, about 2.0-fold, about 3.0-fold, about 3.5-fold, about 4.5-fold, about 5.0-fold, about 10-fold, about 15-fold, about 20-fold, about 30-fold, about 40-fold, about 50-fold, about 100-fold, about 1000-fold, or more). A level of a protein may be expressed in mass/vol (e.g., g/dL, mg/mL, μg/mL, or ng/mL) or percentage relative to total protein in a sample.

As used herein, “alternative lengthening of telomeres” and “ALT” refer to a recombination-based mechanism of telomere maintenance characterized by heterogeneous fluctuating telomere lengths, high levels of telomere sister chromatid exchanges, abundant extrachromosomal telomeric repeat DNA, and specialized telomeric DNA nuclear structures termed ALT-associated promyelocytic leukemia bodies. ALT can also be described as telomerase-independent telomere maintenance. ALT refers to a mechanism of maintaining the length of telomeres (preventing telomere shortening) that is independent of the activity of telomerase. An “ALT-positive cancer” refers to a cancer that displays or possesses ALT activity, or is characterized by the activation of the ALT mechanism. Methods of determining the ALT status of a tumor are well known in the art and can involve combined promyelocytic leukemia (PML) immunofluorescence/telomere fluorescence in situ hybridization (TEL-FISH), analysis of tumor sections for ALT-associated PML bodies or C-circle detection (e.g., by q_PCR).

As used herein, the term “SMARCAL1” refers to SWI/SNF-related matrix-associated actin-dependent regulator of chromatin subfamily A-like protein 1, an ATP-dependent annealing helicase, and a member of the SWI/SNF family of proteins. SMARCAL1 is involved in restarting stalled replication forks by catalyzing branch migration and fork regression. SMARCAL1 is encoded by the SMARCAL1 gene. The amino acid sequence of an exemplary protein encoded by human SMARCAL1 is shown under UniProt Accession No. Q9NZC9 or in SEQ ID NO: 1. The nucleic acid sequence of an exemplary human SMARCAL1 is shown under NCBI Reference Sequence: NM_001127207.1 or in SEQ ID NO: 2. The term “SMARCAL1” also refers to natural variants of the wild-type SMARCAL1 protein, such as proteins having at least 85% identity (e.g., 85%, 86%, 87%, 88%, 89%, 90%, 91%, 92%, 93%, 94%, 95%, 96%, 97%, 98%, 99%, 99.9% identity, or more) to the amino acid sequence of wild-type SMARCAL1, an example of which is set forth in SEQ ID NO: 1.

By “reducing the activity of SMARCAL1” is meant decreasing the level of an activity related to a SMARCAL1, or a related downstream effect. The activity level of SMARCAL1 may be measured using any method known in the art. In some embodiments, an agent which reduces the activity of SMARCAL1 is a polynucleotide. In some embodiments, an agent which reduces the activity of SMARCAL1 is a nuclease.

By “reducing the level of SMARCAL1” is meant decreasing the level of SMARCAL1 in a cell or subject, e.g., by administering a polynucleotide to the cell or subject. The level of SMARCAL1 may be measured using any method known in the art.

As used herein, the term “ATRX” refers to alpha-thalassemia/mental retardation, X-linked, a member of the SWI/SNF family of chromatin-remodeling proteins. ATRX is involved in histone H3.3 deposition at telomeres and other genomic repeats. ATRX is encoded by the ATRX gene. The amino acid sequence of an exemplary protein encoded by human ATRX is shown under UniProt Accession No. P46100 or in SEQ ID NO: 3. The nucleic acid sequence of an exemplary human ATRX is shown under NCBI Reference Sequence: NM_000489.5 or in SEQ ID NO: 4. The term “ATRX” also refers to natural variants of the wild-type ATRX protein, such as proteins having at least 85% identity (e.g., 85%, 86%, 87%, 88%, 89%, 90%, 91%, 92%, 93%, 94%, 95%, 96%, 97%, 98%, 99%, 99.9% identity, or more) to the amino acid sequence of wild-type ATRX, an example of which is set forth in SEQ ID NO: 3.

As used herein, the term “DAXX” refers to death domain associated protein, an H3.3 histone chaperone. DAXX is encoded by the DAXX gene. The amino acid sequence of an exemplary protein encoded by human DAXX is shown under UniProt Accession No. Q9UER7 or in SEQ ID NO: 5. The nucleic acid sequence of an exemplary human DAXX is shown under NCBI Reference Sequence: NM_001141970.1 or in SEQ ID NO: 6. The term “DAXX” also refers to natural variants of the wild-type DAXX protein, such as proteins having at least 85% identity (e.g., 85%, 86%, 87%, 88%, 89%, 90%, 91%, 92%, 93%, 94%, 95%, 96%, 97%, 98%, 99%, 99.9% identity, or more) to the amino acid sequence of wild-type DAXX, which is set forth in SEQ ID NO: 5.

As used herein, the terms “SMARCAL1 inhibitor” and “anti-SMARCAL1 agent” refer to any agent which reduces the level and/or activity of SMARCAL1. Non-limiting examples of anti-SMARCAL1 agents include nucleases, polynucleotides (e.g., siRNA), small-molecule compounds, antibodies, and enzymes.

As used herein, the terms “effective amount,” “therapeutically-effective amount,” and “a “sufficient amount” of an agent that reduces the level and/or activity of SMARCAL1 in a cell in a subject described herein refer to a quantity sufficient to, when administered to the subject, including a human, effect beneficial or desired results, including clinical results, and, as such, an “effective amount” or synonym thereto depends on the context in which it is being applied. For example, in the context of treating cancer, it is an amount of the agent that reduces the level and/or activity of SMARCAL1 in a cell in a subject sufficient to achieve a treatment response as compared to the response obtained without administration of the agent that reduces the level and/or activity of SMARCAL1. The amount of a given agent that reduces the level and/or activity of SMARCAL1 described herein that will correspond to such an amount will vary depending upon various factors, such as the given agent, the pharmaceutical formulation, the route of administration, the type of disease or disorder, the identity of the subject (e.g., age, sex, and/or weight) or host being treated, and the like, but can nevertheless be routinely determined by one of skill in the art. Also, as used herein, a “therapeutically-effective amount” of an agent that reduces the level and/or activity of SMARCAL1 of the present disclosure is an amount which results in a beneficial or desired result in a subject as compared to a control. As defined herein, a therapeutically-effective amount of an agent that reduces the level and/or activity of SMARCAL1 of the present disclosure may be readily determined by one of ordinary skill by routine methods known in the art. Dosage regimen may be adjusted to provide the optimum therapeutic response. As used herein, the term “reducing tumor growth” refers to an inhibition or a reduction in tumor growth or metastasis of a cancer as compared to its growth prior to treatment. The reduction of tumor growth may be a reduction of about 5% or greater (e.g., 5%, 10%, 20%, 30%, 40%, 50%, 60%, 70%, 80%, 90%, or greater), and can be measured by any suitable means known in the art.

The term “inhibitory RNA agent” refers to an RNA, or analog thereof, having sufficient sequence complementarity to a target RNA to direct RNA interference. Examples also include a DNA that can be used to make the RNA. RNA interference (RNAi) refers to a sequence-specific or selective process by which a target molecule (e.g., a target gene, protein, or RNA) is down-regulated. Generally, an interfering RNA (“iRNA”) is a double-stranded short-interfering RNA (siRNA), short hairpin RNA (shRNA), or single-stranded micro-RNA (miRNA) that results in catalytic degradation of specific mRNAs, and also can be used to lower or inhibit gene expression.

The terms “short interfering RNA” and “siRNA” (also known as “small interfering RNAs”) refer to an RNA agent, preferably a double-stranded agent, of about 10-50 nucleotides in length, the strands optionally having overhanging ends comprising, for example 1, 2 or 3 overhanging nucleotides (or nucleotide analogs), which is capable of directing or mediating RNA interference. Naturally-occurring siRNAs are generated from longer dsRNA molecules (e.g., >25 nucleotides in length) by a cell's RNAi machinery (e.g., Dicer or a homolog thereof).

The term “shRNA,” as used herein, refers to an RNA agent having a stem-loop structure, comprising a first and second region of complementary sequence, the degree of complementarity and orientation of the regions being sufficient such that base pairing occurs between the regions, the first and second regions being joined by a loop region, the loop resulting from a lack of base pairing between nucleotides (or nucleotide analogs) within the loop region.

The terms “miRNA” and “microRNA” refer to an RNA agent, preferably a single-stranded agent, of about 10-50 nucleotides in length, preferably between about 15-25 nucleotides in length, which is capable of directing or mediating RNA interference. Naturally-occurring miRNAs are generated from stem-loop precursor RNAs (i.e., pre-miRNAs) by Dicer. The term “Dicer,” as used herein, includes Dicer as well as any Dicer ortholog or homolog capable of processing dsRNA structures into siRNAs, miRNAs, siRNA-like or miRNA-like molecules. The term microRNA (“miRNA”) is used interchangeably with the term “small temporal RNA” (“stRNA”) based on the fact that naturally-occurring miRNAs have been found to be expressed in a temporal fashion (e.g., during development).

The term “antisense,” as used herein, refers to a nucleic acid comprising a polynucleotide that is sufficiently complementary to all or a portion of a gene, primary transcript, or processed mRNA, so as to interfere with expression of the endogenous gene (e.g., SMARCAL1). “Complementary” polynucleotides are those that are capable of base pairing according to the standard Watson-Crick complementarity rules. Specifically, purines will base pair with pyrimidines to form a combination of guanine paired with cytosine (G:C) and adenine paired with either thymine (A:T) in the case of DNA, or adenine paired with uracil (A:U) in the case of RNA. It is understood that two polynucleotides may hybridize to each other even if they are not completely complementary to each other, provided that each has at least one region that is substantially complementary to the other.

The term “antisense nucleic acid” includes single-stranded RNA as well as double-stranded DNA expression cassettes that can be transcribed to produce an antisense RNA. “Active” antisense nucleic acids are antisense RNA molecules that are capable of selectively hybridizing with a primary transcript or mRNA encoding a polypeptide having at least 80% sequence identity (e.g., 80%, 85%, 86%, 87%, 88%, 89%, 90%, 91%, 92%, 93%, 94%, 95%, 96%, 97%, 98%, 99%, 99.9% identity, or more) with the targeted polypeptide sequence (e.g., a SMARCAL1 polypeptide sequence). The antisense nucleic acid can be complementary to an entire coding strand, or to only a portion thereof. In some embodiments, an antisense nucleic acid molecule is antisense to a “coding region” of the coding strand of a nucleotide sequence. The term “coding region” refers to the region of the nucleotide sequence comprising codons that are translated into amino acid residues. In some embodiments, the antisense nucleic acid molecule is antisense to a “noncoding region” of the coding strand of a nucleotide sequence. The term “noncoding region” refers to 5′ and 3′ sequences that flank the coding region that are not translated into amino acids (i.e., also referred to as 5′ and 3′ untranslated regions). The antisense nucleic acid molecule can be complementary to the entire coding region of mRNA, or can be antisense to only a portion of the coding or noncoding region of an mRNA. For example, the antisense oligonucleotide can be complementary to the region surrounding the translation start site. An antisense oligonucleotide can be, for example, about 5, 10, 15, 20, 25, 30, 35, 40, 45, or 50 nucleotides in length.

“Percent (%) sequence identity,” with respect to a reference polynucleotide or polypeptide sequence, is defined as the percentage of nucleic acids or amino acids in a candidate sequence that are identical to the nucleic acids or amino acids in the reference polynucleotide or polypeptide sequence, after aligning the sequences and introducing gaps, if necessary, to achieve the maximum percent sequence identity. Alignment for purposes of determining percent nucleic acid or amino acid sequence identity can be achieved in various ways that are within the capabilities of one of skill in the art, for example, using publicly available computer software, such as BLAST, BLAST-2, or Megalign software. Those skilled in the art can determine appropriate parameters for aligning sequences, including any algorithms needed to achieve maximal alignment over the full length of the sequences being compared. For example, percent sequence identity values may be generated using the sequence comparison computer program BLAST. As an illustration, the percent sequence identity of a given nucleic acid or amino acid sequence, A, to, with, or against a given nucleic acid or amino acid sequence, B, (which can alternatively be phrased as a given nucleic acid or amino acid sequence, A that has a certain percent sequence identity to, with, or against a given nucleic acid or amino acid sequence, B) is calculated as follows:

100 multiplied by (the fraction X/Y)

where X is the number of nucleotides or amino acids scored as identical matches by a sequence alignment program (e.g., BLAST) in that program's alignment of A and B, and where Y is the total number of nucleic acids in B. It will be appreciated that where the length of nucleic acid or amino acid sequence A is not equal to the length of nucleic acid or amino acid sequence B, the percent sequence identity of A to B will not equal the percent sequence identity of B to A.

As used herein, the term “sample” refers to a specimen (e.g., a tissue sample (e.g., a tumor tissue sample), cells, urine, blood, saliva, amniotic fluid, or cerebrospinal fluid) isolated from a subject.

By a “reference” is meant any useful reference used to compare protein or mRNA levels or activity. The reference can be any sample, standard, standard curve, or level that is used for comparison purposes. The reference can be a normal reference sample or a reference standard or level. A “reference sample” can be, for example, a control, e.g., a predetermined negative control value, such as a “normal control” or a prior sample taken from the same subject; a sample from a normal healthy subject, such as a normal cell or normal tissue; a sample (e.g., a cell or tissue) from a subject not having a disease; a sample from a subject that is diagnosed with a disease, but not yet treated with a therapeutic agent described herein; a sample from a subject that has been treated by a therapeutic agent described herein; or a sample of a purified protein (e.g., any described herein) at a known normal concentration. By “reference standard or level” is meant a value or number derived from a reference sample. A “normal control value” is a pre-determined value indicative of non-disease state, e.g., a value expected in a healthy control subject. Typically, a normal control value is expressed as a range (“between X and Y”), a high threshold (“no higher than X”), or a low threshold (“no lower than X”). A subject having a measured value within the normal control value for a particular biomarker is typically referred to as “within normal limits” for that biomarker. A normal reference standard or level can be a value or number derived from a normal subject not having a disease or disorder (e.g., cancer); or a subject that has been treated with a therapeutic agent described herein. In particular embodiments, the reference sample, standard, or level is matched to the sample subject sample by at least one of the following criteria: age, weight, sex, disease stage, and overall health. A standard curve of levels of a purified protein, e.g., as described herein, within the normal reference range can also be used as a reference.

As used interchangeably herein, the terms “subject,” “patient,” and “individual” refer to any organism to which a therapeutic agent in accordance with the invention may be administered, e.g., for experimental, diagnostic, prophylactic, and/or therapeutic purposes. Typical subjects include any animal (e.g., mammals, such as mice, rats, rabbits, non-human primates, and humans). A subject may seek or be in need of treatment, require treatment, be receiving treatment, be receiving treatment in the future, or be a human or animal who is under care by a trained professional for a particular disease or condition.

As used herein, the terms “treat,” “treated,” and “treating” mean both therapeutic treatment and prophylactic or preventative measures wherein the object is to prevent or slow down (lessen) an undesired physiological condition, disorder, or disease, or obtain beneficial or desired clinical results. Beneficial or desired clinical results include, but are not limited to, alleviation of symptoms; diminishment of the extent of a condition, disorder, or disease; stabilization of the (i.e., not worsening) state of condition, disorder, or disease; delay in onset or slowing of condition, disorder, or disease progression; amelioration of the condition, disorder, or disease state or remission (whether partial or total), whether detectable or undetectable; an amelioration of at least one measurable physical parameter, not necessarily discernible by the patient; or enhancement or improvement of condition, disorder, or disease. Treatment includes eliciting a clinically-significant response without excessive levels of side effects. Treatment also includes prolonging survival as compared to expected survival if not receiving treatment.

The term “telomerase inhibitor,” as used herein, refers to a compound, such as a peptide or a vaccine capable of inhibiting the activity of the protein that in humans is encoded by the TERT gene (UniProt Reference No. O14746). Known telomerase inhibitors include RIAVAX™ (tertomotide) and imetelstat.

The details of one or more embodiments of the invention are set forth in the description below. Other features, objects, and advantages of the invention will be apparent from the description and from the claims.

### BRIEF DESCRIPTION OF THE DRAWINGS

FIG. 1 is an illustration of a PARIS correlation analysis between ATRX deficiency status and gene dependency by CRISPR knock down.

FIG. 2A and FIG. 2B are a pair of graphs illustrating the effect of sgRNA targeting of the SMARCAL1 on G-292 (FIG. 2A) and SAOS-2 (FIG. 2B) osteosarcoma cell growth. The Y-axis indicates the dropout ratio. The X-axis indicates the nucleotide position of the SMARCAL1 open reading frame. The grey box indicates the range of the negative control sgRNAs in the screen. The linear protein sequence is show with SMARCAL1 PFAM domains annotated from the PFAM database.

FIG. 3 is a graph illustrating SMARCAL1 genetic dependency in ATRX null/ALT+osteosarcoma cell line SAOS2.

FIG. 4 is an image illustrating SMARCAL1 genetic dependency in ATRX null/ALT+osteosarcoma cell line CAL72.

FIG. 5 is an image illustrating SMARCAL1 genetic dependency in ATRX null/ALT+astrocytoma cell line TM31.

FIG. 6 is an image illustrating SMARCAL1 genetic dependency in alveolar rhadomyosarcoma cell line RH30 and ATRX null fibroblast cell line HS729.

### DETAILED DESCRIPTION

The present inventors have found that reducing the level and/or activity of the SWI/SNF-related matrix-associated actin-dependent regulator of chromatin subfamily A-like protein 1 (SMARCAL1) in ALT-positive cancer cells and/or in cancer cells having a mutation that results in a loss of function alpha-thalassemia/mental retardation, X-linked (ATRX) and/or death domain associated protein (DAXX) inhibits the proliferation of the cancer cells. Accordingly, the invention features methods for reducing the level and/or activity of SMARCAL1 for the treatment of cancer, e.g., in a subject in need thereof. Exemplary methods are described herein.

### ALT and Cancer

Alternative lengthening of telomeres (ALT) is a recombination—based mechanism of telomere maintenance use by 10-15% of human cancers. ALT activity is most prevalent in cancers arising from mesenchymal tissues, including bone (62%), soft tissues (32%), neuroendocrine systems (40%), peripheral nervous systems (23%), and central nervous system (15%). ALT is characterized by heterogeneous fluctuating telomere lengths, high levels of telomere sister chromatid exchanges, abundant extrachromosomal telomeric repeat DNA, and specialized telomeric DNA nuclear structures termed ALT-associated promyelocytic leukemia bodies.

ALT status of a tumor may be assessed either by combined promyelocytic leukemia (PML) immunofluorescence/telomere fluorescence in situ hybridization (TEL-FISH) analysis of tumor sections for ALT-associated PML bodies or C-circle detection (e.g., by qPCR). Methods of determining ALT status of a tumor are well known in the art.

### SMARCAL1

SWI/SNF-related matrix-associated actin-dependent regulator of chromatin subfamily A-like protein 1 (SMARCAL1) is an ATP-dependent annealing helicase that functions to restart stalled replication forks by catalyzing branch migration and fork regression. It is a member of the SWI/SNF family of chromatin-remodeling proteins, which have helicase and ATPase activities. SMARCAL1 plays a role in the DNA damage response as well as resolving endogenous DNA replication stress (e.g., from telomeres). SMARCAL1 converts Replication Protein A (RPA)-bound, single stranded DNA into double-stranded DNA, an enzyme activity termed “annealing helicase.”

Defects in the SMARCAL1 gene cause Schimke immunoosseous dysplasia, a condition characterized by short stature, kidney disease, and a weakened immune system. An example of wild-type human SMARCAL1 (UNIPROT reference number: Q9NZC9) has the amino acid sequence of:

(SEQ ID NO: 1)
MSLPLTEEQRKKIEENRQKALARRAEKLLAEQHQRTSSGTSIAGNPFQAKQ
GPSQNFPRESCKPVSHGVIFKQQNLSSSSNADQRPHDSHSFQAKGIWKKPE
EMPTACPGHSPRSQMALTGISPPLAQSPPEVPKQQLLSYELGQGHAQASPE
IRFTPFANPTHKPLAKPKSSQETPAHSSGQPPRDAKLEAKTAKASPSGQNI
SYIHSSSESVTPRTEGRLQQKSGSSVQKGVNSQKGKCVRNGDRFQVLIGYN
AELIAVFKTLPSKNYDPDTKTWNFSMNDYSALMKAAQSLPTVNLQPLEWAY
GSSESPSTSSEGQAGLPSAPSLSFVKGRCMLISRAYFEADISYSQDLIALF
KQMDSRRYDVKTRKWSFLLEEHSKLIAKVRCLPQVQLDPLPTTLTLAFASQ
LKKTSLSLTPDVPEADLSEVDPKLVSNLMPFQRAGVNFAIAKGGRLLLADD
MGLGKTIQAICIAAFYRKEWPLLVVVPSSVRFTWEQAFLRWLPSLSPDCIN
VVVTGKDRLTAGLINIVSFDLLSKLEKQLKTPFKVVIIDESHFLKNSRTAR
CRAAMPVLKVAKRVILLSGTPAMSRPAELYTQIIAVKPTFFPQFHAFGLRY
CDAKRMPWGWDYSGSSNLGELKLLLEEAVMLRRLKSDVLSQLPAKQRKIVV
IAPGRINARTRAALDAAAKEMTTKDKTKQQQKDALILFFNRTAEAKIPSVI
EYILDLLESGREKFLVFAHHKVVLDAITQELERKHVQHIRIDGSTSSAERE
DLCQQFQLSERHAVAVLSITAANMGLTFSSADLVVFAELFWNPGVLIQAED
RVHRIGQTSSVGIHYLVAKGTADDYLWPLIQEKIKVLAEAGLSETNFSEMT
ESTDYLYKDPKQQKIYDLFQKSFEKEGSDMELLEAAESFDPGSASGTSGSS
SQNMGDTLDESSLTASPQKKRRFEFFDNWDSFTSPL.

An example of wild-type human SMARCAL1 (GenBank accession number: NM_001127207.1) has the nucleic acid sequence of:

(SEQ ID NO: 2)
GATCGTAGGGGTGGGAGTGGGGCGTGGCGCCCGCTTACCTTGAGGCTGGGT
TGGAAAAAGACTATGTTAGCAAGTGTCACGCCATGCTTTTGCCAACTTTCC
AATTAAAGGTTGACATTCCTGCATAAGCATTTCTCTGTGAAAATGTCCTTG
CCTCTTACAGAGGAGCAGAGGAAAAAGATTGAAGAGAATCGACAAAAGGCT
CTGGCCCGCAGAGCTGAGAAGTTATTGGCAGAACAGCATCAGAGGACTAGC
TCGGGCACCTCCATTGCTGGCAACCCATTCCAGGCCAAGCAAGGCCCATCC
CAAAATTTCCCAAGGGAGTCTTGTAAGCCAGTGAGCCATGGTGTCATTTTC
AAGCAACAGAATCTCAGTAGCTCATCTAATGCTGACCAAAGACCTCATGAT
TCCCACAGTTTTCAGGCAAAGGGAATATGGAAAAAGCCAGAAGAAATGCCC
ACAGCCTGCCCAGGCCACAGTCCACGTAGTCAAATGGCTCTCACTGGAATC
TCTCCTCCCTTGGCACAAAGTCCTCCAGAGGTCCCTAAACAACAGCTCTTG
AGTTATGAGTTAGGTCAAGGTCATGCTCAGGCTTCACCTGAGATCAGGTTC
ACACCCTTTGCTAACCCAACTCATAAGCCTCTGGCCAAACCAAAGAGTTCC
CAAGAGACACCAGCTCATTCCTCTGGACAGCCTCCCAGGGATGCTAAGTTA
GAGGCCAAGACAGCAAAAGCCTCCCCTTCGGGGCAGAACATTTCTTACATC
CATTCTAGCTCAGAGAGTGTAACGCCCAGGACAGAAGGAAGACTCCAGCAG
AAGTCAGGGTCCTCAGTCCAAAAAGGAGTGAACTCTCAGAAGGGAAAGTGC
GTAAGGAACGGCGATCGTTTCCAGGTGTTGATTGGGTACAATGCGGAACTC
ATTGCAGTGTTTAAGACCCTGCCCAGCAAGAATTATGATCCTGACACCAAG
ACGTGGAACTTCAGCATGAATGACTATAGTGCCCTGATGAAAGCAGCCCAG
AGCCTCCCCACGGTCAACCTGCAGCCTCTGGAATGGGCCTATGGCAGCAGC
GAGTCACCCTCCACCAGCAGTGAGGGACAGGCCGGCCTTCCATCAGCTCCA
TCCCTTTCATTTGTCAAAGGGCGATGCATGCTCATCTCCAGGGCCTACTTC
GAGGCAGACATCAGTTATTCACAGGACCTTATTGCGCTTTTTAAACAGATG
GATTCCAGAAGATATGATGTCAAGACCAGGAAGTGGAGCTTTCTCTTGGAA
GAGCACAGTAAACTAATTGCAAAGGTGCGCTGCCTCCCACAAGTTCAGCTG
GACCCTCTGCCCACGACTCTCACCCTGGCGTTTGCTTCTCAGCTCAAGAAG
ACATCTCTCAGTCTCACGCCAGATGTCCCAGAGGCAGACCTTTCTGAAGTG
GACCCCAAGCTCGTGTCTAATCTGATGCCCTTTCAGAGAGCTGGAGTCAAT
TTTGCCATAGCCAAAGGAGGCCGCCTGCTGCTCGCTGACGACATGGGCCTG
GGGAAGACCATCCAAGCCATCTGCATCGCAGCCTTTTACCGGAAGGAGTGG
CCGCTCCTGGTGGTGGTGCCATCCTCCGTGCGCTTCACCTGGGAGCAGGCC
TTCCTTCGGTGGCTGCCATCTCTGAGCCCAGATTGCATCAACGTCGTGGTG
ACTGGGAAGGACCGCCTGACAGCTGGCCTGATCAACATTGTCAGCTTTGAC
CTTCTTAGCAAGTTGGAAAAACAGCTAAAAACCCCTTTTAAAGTTGTCATC
ATTGATGAATCTCACTTCCTCAAAAACAGTAGGACTGCCCGCTGTCGAGCA
GCTATGCCGGTCCTAAAGGTTGCCAAGAGGGTGATCCTGTTGTCGGGCACA
CCAGCCATGTCCCGGCCCGCAGAGCTCTACACGCAGATCATCGCAGTCAAG
CCAACTTTCTTCCCCCAGTTTCATGCCTTTGGACTTCGCTACTGTGATGCC
AAACGGATGCCTTGGGGGTGGGACTACTCAGGTTCCTCCAACCTGGGAGAG
CTGAAGCTCCTGCTGGAGGAAGCAGTCATGCTGCGGCGCCTCAAGTCCGAC
GTCCTTTCCCAGCTGCCTGCCAAGCAGCGCAAGATAGTGGTGATTGCCCCA
GGACGGATCAATGCCAGGACCAGAGCTGCCCTGGATGCTGCAGCCAAGGAA
ATGACCACCAAGGACAAAACTAAACAGCAGCAGAAAGATGCCCTCATTCTC
TTCTTCAACAGAACAGCTGAAGCTAAAATCCCATCTGTCATTGAATATATC
TTGGACCTACTGGAAAGTGGAAGAGAGAAGTTTTTAGTATTTGCACACCAT
AAGGTGGTCCTGGACGCAATTACGCAAGAGCTTGAGAGAAAGCACGTGCAG
CACATCCGCATCGATGGCTCCACCTCATCAGCTGAGCGGGAGGACCTGTGC
CAGCAGTTCCAACTGTCGGAGAGGCATGCTGTGGCCGTGCTGTCCATCACC
GCTGCCAATATGGGCCTCACCTTCTCCTCGGCTGACCTGGTGGTGTTTGCT
GAGCTGTTTTGGAACCCAGGGGTGCTGATCCAGGCTGAGGACCGCGTGCAC
CGCATTGGACAGACCAGCTCCGTGGGCATTCACTACCTCGTGGCAAAGGGC
ACAGCTGATGACTACCTTTGGCCCCTGATTCAAGAGAAGATTAAAGTTCTG
GCAGAAGCCGGGCTTTCTGAGACCAATTTTTCAGAAATGACAGAATCCACT
GATTACCTCTACAAGGACCCAAAGCAGCAGAAGATCTACGACCTATTCCAG
AAGTCCTTTGAGAAAGAAGGAAGTGATATGGAGCTCCTGGAAGCAGCAGAG
TCCTTTGACCCAGGAAGTGCTTCAGGAACATCTGGAAGTAGTTCCCAGAAC
ATGGGAGACACCCTGGATGAAAGCTCATTGACAGCCAGTCCACAGAAGAAA
AGGAGATTTGAATTTTTTGATAACTGGGACAGCTTTACGTCTCCCCTGTAA
AAGGGGCAAAAAGAAAAAAATAAAAAGCATTTTAAAATCATGGAATTGAAA
TAAAATAATGTATTTTGTTTTAAAAAAAAAAAAAA.

### ATRX and DAXX

ATRX is a member of the SWI/SNF family of chromatin-remodeling proteins. ATRX is required for deposition of the histone variant H3.3 at telomeres and other genomic repeats. These interactions are important for maintaining silencing at these sites. ATRX also undergoes cell cycle-dependent phosphorylation, which regulates its nuclear matrix and chromatin association, and suggests its involvement in the gene regulation at interphase and chromosomal segregation in mitosis. Inherited mutations of the ATRX gene are associated with an X-linked mental retardation syndrome most often accompanied by alpha-thalassemia syndrome. Acquired mutations in ATRX have also been reported in a number of human cancers including pancreatic neuroendocrine tumors, gliomas, astrocytomas, osteosarcomas, and malignant pheochromocytomas.

DAXX functions as an H3.3-specific histone chaperone, interacting with an H3.3/H4 dimer. It interacts with a wide variety of proteins, including the apoptosis antigen Fas, centromere protein C, and transcription factor erythroblastosis virus E26 oncogene homolog 1. In the nucleus, the encoded protein functions as a potent transcription repressor that binds to sumoylated transcription factors. Its repression can be relieved by the sequestration of this protein into promyelocytic leukemia nuclear bodies or nucleoli. DAXX also associates with centromeres in G2 phase. In the cytoplasm, the encoded protein may function to regulate apoptosis. The subcellular localization and function of this protein are modulated by post-translational modifications, including sumoylation, phosphorylation, and polyubiquitination.

Together, ATRX and DAXX form a complex involved in depositing the histone H3.3 at pericentric heterochromatin and at telomeres. Mutations in the ATRX/DAXX/H3.3 axis have been identified in ALT-positive tumors. There are no targeted anti-cancer therapies against tumors having mutations in ATRX and/or DAXX.

An example of wild-type human ATRX (UNIPROT reference number: P46100) has the amino acid sequence of:

(SEQ ID NO: 3)
MTAEPMSESKLNTLVQKLHDFLAHSSEESEETSSPPRLAMNQNTDKISGSG
SNSDMMENSKEEGTSSSEKSKSSGSSRSKRKPSIVTKYVESDDEKPLDDET
VNEDASNENSENDITMQSLPKGTVIVQPEPVLNEDKDDFKGPEFRSRSKMK
TENLKKRGEDGLHGIVSCTACGQQVNHFQKDSIYRHPSLQVLICKNCFKYY
MSDDISRDSDGMDEQCRWCAEGGNLICCDFCHNAFCKKCILRNLGRKELST
IMDENNQWYCYICHPEPLLDLVTACNSVFENLEQLLQQNKKKIKVDSEKSN
KVYEHTSRFSPKKTSSNCNGEEKKLDDSCSGSVTYSYSALIVPKEMIKKAK
KLIETTANMNSSYVKFLKQATDNSEISSATKLRQLKAFKSVLADIKKAHLA
LEEDLNSEFRAMDAVNKEKNTKEHKVIDAKFETKARKGEKPCALEKKDISK
SEAKLSRKQVDSEHMHQNVPTEEQRTNKSTGGEHKKSDRKEEPQYEPANTS
EDLDMDIVSVPSSVPEDIFENLETAMEVQSSVDHQGDGSSGTEQEVESSSV
KLNISSKDNRGGIKSKTTAKVTKELYVKLTPVSLSNSPIKGADCQEVPQDK
DGYKSCGLNPKLEKCGLGQENSDNEHLVENEVSLLLEESDLRRSPRVKTTP
LRRPTETNPVTSNSDEECNETVKEKQKLSVPVRKKDKRNSSDSAIDNPKPN
KLPKSKQSETVDQNSDSDEMLAILKEVSRMSHSSSSDTDINEIHTNHKTLY
DLKTQAGKDDKGKRKRKSSTSGSDFDTKKGKSAKSSIISKKKRQTQSESSN
YDSELEKEIKSMSKIGAARTTKKRIPNTKDFDSSEDEKHSKKGMDNQGHKN
LKTSQEGSSDDAERKQERETFSSAEGTVDKDTTIMELRDRLPKKQQASAST
DGVDKLSGKEQSFTSLEVRKVAETKEKSKHLKTKTCKKVQDGLSDIAEKFL
KKDQSDETSEDDKKQSKKGTEEKKKPSDFKKKVIKMEQQYESSSDGTEKLP
EREEICHFPKGIKQIKNGTTDGEKKSKKIRDKTSKKKDELSDYAEKSTGKG
DSCDSSEDKKSKNGAYGREKKRCKLLGKSSRKRQDCSSSDTEKYSMKEDGC
NSSDKRLKRIELRERRNLSSKRNTKEIQSGSSSSDAEESSEDNKKKKQRTS
SKKKAVIVKEKKRNSLRTSTKRKQADITSSSSSDIEDDDQNSIGEGSSDEQ
KIKPVTENLVLSSHTGFCQSSGDEALSKSVPVTVDDDDDDNDPENRIAKKM
LLEEIKANLSSDEDGSSDDEPEEGKKRTGKQNEENPGDEEAKNQVNSESDS
DSEESKKPRYRHRLLRHKLTVSDGESGEEKKTKPKEHKEVKGRNRRKVSSE
DSEDSDFQESGVSEEVSESEDEQRPRTRSAKKAELEENQRSYKQKKKRRRI
KVQEDSSSENKSNSEEEEEEKEEEEEEEEEEEEEEEDENDDSKSPGKGRKK
IRKILKDDKLRTETQNALKEEEERRKRIAEREREREKLREVIEIEDASPTK
CPITTKLVLDEDEETKEPLVQVHRNMVIKLKPHQVDGVQFMWDCCCESVKK
TKKSPGSGCILAHCMGLGKTLQVVSFLHTVLLCDKLDFSTALVVCPLNTAL
NWMNEFEKWQEGLKDDEKLEVSELATVKRPQERSYMLQRWQEDGGVMIIGY
EMYRNLAQGRNVKSRKLKEIFNKALVDPGPDFVVCDEGHILKNEASAVSKA
MNSIRSRRRIILTGTPLQNNLIEYHCMVNFIKENLLGSIKEFRNRFINPIQ
NGQCADSTMVDVRVMKKRAHILYEMLAGCVQRKDYTALTKFLPPKHEYVLA
VRMTSIQCKLYQYYLDHLTGVGNNSEGGRGKAGAKLFQDFQMLSRIWTHPW
CLQLDYISKENKGYFDEDSMDEFIASDSDETSMSLSSDDYTKKKKKGKKGK
KDSSSSGSGSDNDVEVIKVWNSRSRGGGEGNVDETGNNPSVSLKLEESKAT
SSSNPSSPAPDWYKDFVTDADAEVLEHSGKMVLLFEILRMAEEIGDKVLVF
SQSLISLDLIEDFLELASREKTEDKDKPLIYKGEGKWLRNIDYYRLDGSTT
AQSRKKWAEEFNDETNVRGRLFIISTKAGSLGINLVAANRVIIFDASWNPS
YDIQSIFRVYRFGQTKPVYVYRFLAQGTMEDKIYDRQVTKQSLSFRVVDQQ
QVERHFTMNELTELYTFEPDLLDDPNSEKKKKRDTPMLPKDTILAELLQIH
KEHIVGYHEHDSLLDHKEEEELTEEERKAAWAEYEAEKKGLTMRFNIPTGT
NLPPVSFNSQTPYIPFNLGALSAMSNQQLEDLINQGREKVVEATNSVTAVR
IQPLEDIISAVWKENMNLSEAQVQALALSRQASQELDVKRREAlYNDVLTK
QQMLISCVQRILMNRRLQQQYNQQQQQQMTYQQATLGHLMMPKPPNLIMNP
SNYQQIDMRGMYQPVAGGMQPPPLQRAPPPMRSKNPGPSQGKSM.

An example of wild-type human DAXX (UNIPROT reference number: Q9UER7) has the amino acid sequence of:

(SEQ ID NO: 5)
MATANSIIVLDDDDEDEAAAQPGPSHPLPNAASPGAEAPSSSEPHGARGSS
SSGGKKCYKLENEKLFEEFLELCKMQTADHPEVVPFLYNRQQRAHSLFLAS
AEFCNILSRVLSRARSRPAKLYVYINELCTVLKAHSAKKKLNLAPAATTSN
EPSGNNPPTHLSLDPTNAENTASQSPRTRGSRRQIQRLEQLLALYVAEIRR
LQEKELDLSELDDPDSAYLQEARLKRKLIRLFGRLCELKDCSSLTGRVIEQ
RIPYRGTRYPEVNRRIERLINKPGPDTFPDYGDVLRAVEKAAARHSLGLPR
QQLQLMAQDAFRDVGIRLQERRHLDLIYNFGCHLTDDYRPGVDPALSDPVL
ARRLRENRSLAMSRLDEVISKYAMLQDKSEEGERKKRRARLQGTSSHSADT
PEASLDSGEGPSGMASQGCPSASRAETDDEDDEESDEEEEEEEEEEEEEAT
DSEEEEDLEQMQEGQEDDEEEDEEEEAAAGKDGDKSPMSSLQISNEKNLEP
GKQISRSSGEQQNKGRIVSPSLLSEEPLAPSSIDAESNGEQPEELTLEEES
PVSQLFELEIEALPLDTPSSVETDISSSRKQSEEPFTTVLENGAGMVSSTS
FNGGVSPHNWGDSGPPCKKSRKEKKQTGSGPLGNSYVERQRSVHEKNGKKI
CTLPSPPSPLASLAPVADSSTRVDSPSHGLVTSSLCIPSPARLSQTPHSQP
PRPGTCKTSVATQCDPEEIIVLSDSD.

Anti-SMARCAL1 agents

Agents described herein that reduce the level and/or activity of SMARCAL1 in a cell in a subject may be, for example, a polynucleotide, a small-molecule compound, an antibody, and/or an enzyme. The agents reduce the level of SMARCAL1, or reduce the level of an activity related to SMARCAL1 (e.g., SMARCAL1 helicase activity), and/or related downstream effect in a cell or subject. In some embodiments, the agents reduce or inhibit SMARCAL1 helicase activity.

In some embodiments, the agent that reduces the level and/or activity of SMARCAL1 in a cell in a subject is a polynucleotide, a small-molecule compound, an antibody, and/or an enzyme (e.g., a nuclease).

Polynucleotides

In some embodiments, the agent that reduces the level and/or activity of SMARCAL1 is a polynucleotide. In some embodiments, the agent that reduces the level and/or activity of SMARCAL1 is an inhibitory RNA molecule, e.g., that acts by way of the RNA interference (RNAi) pathway. An inhibitory RNA molecule can decrease the expression level (e.g., protein level or mRNA level) of SMARCAL1. For example, an inhibitory RNA molecule includes a short interfering RNA (siRNA), a short hairpin RNA (shRNA), and/or a microRNA (miRNA) that targets full-length SMARCAL1. A siRNA is a double-stranded RNA molecule that typically has a length of about 19-25 base pairs. A shRNA is a RNA molecule including a hairpin turn that decreases expression of target genes via RNAi. A miRNA is a non-coding RNA molecule that typically has a length of about 22 nucleotides. miRNAs bind to target sites on mRNA molecules and silence the mRNA, e.g., by causing cleavage of the mRNA, destabilization of the mRNA, and/or inhibition of translation of the mRNA. Degradation is catalyzed by an enzymatic, RNA-induced silencing complex (RISC).

In some embodiments, the agent that reduces the level and/or activity of SMARCAL1 in a cell in a subject is an antisense nucleic acid. Antisense nucleic acids include antisense RNA (asRNA) and antisense DNA (asDNA) molecules, typically about 10 to 30 nucleotides in length, which recognize polynucleotide target sequences or sequence portions through hydrogen bonding interactions with the nucleotide bases of the target sequences (e.g., SMARCAL1). The target sequences may be single- or double-stranded RNA, or single- or double-stranded DNA.

A polynucleotide of the invention can be modified, e.g., to contain modified nucleotides, e.g., 2′-fluoro, 2′-o-methyl, 2′-deoxy, unlocked nucleic acid, 2′-hydroxy, phosphorothioate, 2′-thiouridine, 4′-thiouridine, 2′-deoxyuridine. Without being bound by theory, it is believed that certain modification can increase nuclease resistance and/or serum stability, or decrease immunogenicity. The polynucleotides mentioned above may also be provided in a specialized form, such as liposomes or microspheres, or may be applied to gene therapy, or may be provided in combination with attached moieties. Such attached moieties include polycations, such as polylysine that act as charge neutralizers of the phosphate backbone, or hydrophobic moieties, such as lipids (e.g., phospholipids, cholesterols, etc.) that enhance the interaction with cell membranes or increase uptake of the nucleic acid. These moieties may be attached to the nucleic acid at the 3′ or 5′ ends and may also be attached through a base, sugar, or intramolecular nucleoside linkage. Other moieties may be capping groups specifically placed at the 3′ or 5′ ends of the nucleic acid to prevent degradation by nucleases, such as exonuclease, RNase, or other nucleases known in the art. Such capping groups include hydroxyl protecting groups known in the art, including glycols, such as polyethylene glycol and tetraethylene glycol. The inhibitory action of the polynucleotide can be examined using a cell-line or animal-based gene expression system of the present invention in vivo and in vitro.

In some embodiments, the polynucleotide decreases the level and/or activity or function of SMARCAL1. In particular embodiments, the polynucleotide inhibits expression of SMARCAL1. In other embodiments, the polynucleotide increases degradation of SMARCAL1 and/or decreases the stability (i.e., half-life) of SMARCAL1. The polynucleotide can be chemically synthesized or transcribed in vitro.

Inhibitory polynucleotides can be designed by methods well known in the art. siRNA, miRNA, shRNA, and asRNA molecules with homology sufficient to provide sequence specificity required to uniquely degrade any RNA can be designed using programs known in the art, including, but not limited to, those maintained on websites for Thermo Fisher Scientific, the German Cancer Research Center, and The Ohio State University Wexner Medical Center. Systematic testing of several designed species for optimization of the inhibitory polynucleotide sequence can be routinely performed by those skilled in the art. Considerations when designing interfering polynucleotides include, but are not limited to, biophysical, thermodynamic, and structural considerations, base preferences at specific positions in the sense strand, and homology. The making and use of inhibitory therapeutic agents based on non-coding RNA, such as ribozymes, RNAse P, siRNAs, and miRNAs are also known in the art, for example, as described in Sioud, RNA Therapeutics: Function, Design, and Delivery (Methods in Molecular Biology). Humana Press 2010. Exemplary inhibitory polynucleotides, for use in the methods of the invention, are provided in Table 1, below. In some embodiments, the inhibitory polynucleotides have a nucleic acid sequence with at least 50% (e.g., at least 50%, at least 60%, at least 70%, at least 80%, at least 85%, at least 90%, at least 95%, at least 96%, at least 97%, at least 98%, at least 99%, or 100%) sequence identity to the nucleic acid sequence of an inhibitory polynucleotide in Table 1. In some embodiments, the inhibitory polynucleotides have a nucleic acid sequence with at least 85% sequence identity to the nucleic acid sequence of an inhibitory polynucleotide in Table 1. In some embodiments, the inhibitory polynucleotides have a nucleic acid sequence with at least 90% sequence identity to the nucleic acid sequence of an inhibitory polynucleotide in Table 1. In some embodiments, the inhibitory polynucleotides have a nucleic acid sequence with at least 95% sequence identity to the nucleic acid sequence of an inhibitory polynucleotide in Table 1.

Construction of vectors for expression of polynucleotides for use in the invention may be accomplished using conventional techniques which do not require detailed explanation to one of ordinary skill in the art. For generation of efficient expression vectors, it is necessary to have regulatory sequences that control the expression of the polynucleotide. These regulatory sequences include promoter and enhancer sequences and are influenced by specific cellular factors that interact with these sequences, and are well known in the art.

Gene Editing

In some embodiments, the agent that reduces the level and/or activity of SMARCAL1 in a cell in a subject is a component of a gene-editing system. For example, the agent that reduces the level and/or activity of SMARCAL1 introduces an alteration (e.g., insertion, deletion (e.g., knockout), translocation, inversion, single point mutation, or other mutation) in SMARCAL1. In some embodiments, the agent that reduces the level and/or activity of SMARCAL1 in a cell in a subject is a nuclease. Gene editing systems include zinc finger nucleases (ZFNs), Transcription Activator-Like Effector-based Nucleases (TALENs), meganucleases, and the clustered regulatory interspaced short palindromic repeat (CRISPR) system. ZFNs, TALENs, and CRISPR-based methods are described, e.g., in Gaj et al., Trends Biotechnol. 31(7):397-405 (2013).

CRISPR refers to a set of (or system including a set of) clustered regularly interspaced short palindromic repeats. A CRISPR system refers to a system derived from CRISPR and Cas (a CRISPR-associated protein) or other nuclease that can be used to silence or mutate a gene described herein. The CRISPR system is a naturally-occurring system found in bacterial and archeal genomes. The CRISPR locus is made up of alternating repeat and spacer sequences. In naturally-occurring CRISPR systems, the spacers are typically sequences that are foreign to the bacterium (e.g., plasmid or phage sequences). The CRISPR system has been modified for use in gene editing (e.g., changing, silencing, and/or enhancing certain genes) in eukaryotes. See, e.g., Wiedenheft et al., Nature 482(7385):331-338 (2012). For example, such modification of the system includes introducing into a eukaryotic cell a plasmid containing a specifically-designed CRISPR and one or more appropriate Cas proteins. The CRISPR locus is transcribed into RNA and processed by Cas proteins into small RNAs that include a repeat sequence flanked by a spacer. The RNAs serve as guides to direct Cas proteins to silence specific

DNA/RNA sequences, depending on the spacer sequence. See, e.g., Horvath et al., Science 327(5962):167-170 (2010); Makarova et al., Biology Direct 1:7 (2006); Pennisi, Science 341(6148):833-836 (2013). In some examples, the CRISPR system includes the Cas9 protein, a nuclease that cuts on both strands of the DNA. See, e.g., Id.

In some embodiments, in a CRISPR system for use described herein, e.g., in accordance with one or more methods described herein, the spacers of the CRISPR are derived from a target gene sequence, e.g., from a SMARCAL1 sequence.

In some embodiments, the agent that reduces the level and/or activity of SMARCAL1 includes a guide RNA (gRNA) for use in a CRISPR system for gene editing. Exemplary gRNAs, for use in the methods of the invention, are provided in Table 1, below. In some embodiments, the agent that reduces the level and/or activity of SMARCAL1 includes a ZFN, or an mRNA encoding a ZFN, that targets (e.g., cleaves) a nucleic acid sequence (e.g., DNA sequence) of SMARCAL1. In some embodiments, the agent that reduces the level and/or activity of SMARCAL1 includes a TALEN, or an mRNA encoding a TALEN, that targets (e.g., cleaves) a nucleic acid sequence (e.g., DNA sequence) of SMARCAL1.

For example, the gRNA can be used in a CRISPR system to engineer an alteration in a gene (e.g., SMARCAL1). In other examples, the ZFN and/or TALEN can be used to engineer an alteration in a gene (e.g., SMARCAL1). Alterations include insertions, deletions (e.g., knockouts), translocations, inversions, single point mutations, and other mutations. The alteration can be introduced in the gene in a cell. In some embodiments, the alteration decreases the level and/or activity of (e.g., knocks down or knocks out) SMARCAL1, e.g., the alteration is a negative regulator of function.

In certain embodiments, the CRISPR system is used to edit (e.g., to add or delete a base pair) a target gene, e.g., SMARCAL1. In other embodiments, the CRISPR system is used to introduce a premature stop codon, e.g., thereby decreasing the expression of a target gene. In yet other embodiments, the CRISPR system is used to turn off a target gene in a reversible manner, e.g., similarly to RNA interference. In further embodiments, the CRISPR system is used to direct Cas to a promoter of a target gene, e.g., SMARCAL1, thereby blocking an RNA polymerase sterically.

In some embodiments, a CRISPR system can be generated to edit SMARCAL1 using technology described in, e.g., U.S. Publication No. 20140068797; Cong et al., Science 339(6121):819-823 (2013); Tsai, Nature Biotechnol., 32(6):569-576 (2014); and U.S. Pat. Nos.: 8,871,445; 8,865,406; 8,795,965; 8,771,945; and 8,697,359.

In some embodiments, the CRISPR interference (CRISPRi) technique can be used for transcriptional repression of specific genes, e.g., the gene encoding SMARCAL1. In CRISPRi, an engineered Cas9 protein (e.g., nuclease-null dCas9, or dCas9 fusion protein, e.g., dCas9-KRAB or dCas9-SID4X fusion) can pair with a sequence-specific guide RNA (sgRNA). The Cas9-gRNA complex can block RNA polymerase, thereby interfering with transcription elongation. The complex can also block transcription initiation by interfering with transcription factor binding. The CRISPRi method is specific with minimal off-target effects and is multiplexable, e.g., can simultaneously repress more than one gene (e.g., using multiple gRNAs). Also, the CRISPRi method permits reversible gene repression.

In some embodiments, CRISPR-mediated gene activation (CRISPRa) can be used for transcriptional activation, e.g., of one or more genes described herein, e.g., a gene that inhibits SMARCAL1. In the CRISPRa technique, dCas9 fusion proteins recruit transcriptional activators. For example, dCas9 can be used to recruit polypeptides (e.g., activation domains), such as VP64, or the p65 activation domain (p65D) and used with sgRNA (e.g., a single sgRNA or multiple sgRNAs), to activate a gene or genes, e.g., endogenous gene(s). Multiple activators can be recruited by using multiple sgRNAs—this can increase activation efficiency. A variety of activation domains and single or multiple activation domains can be used. In addition to engineering dCas9 to recruit activators, sgRNAs can also be engineered to recruit activators. For example, RNA aptamers can be incorporated into a sgRNA to recruit proteins (e.g., activation domains), such as VP64. In some examples, the synergistic activation mediator (SAM) system can be used for transcriptional activation. In SAM, MS2 aptamers are added to the sgRNA. MS2 recruits the MS2 coat protein fused to p65AD and heat shock factor 1. The CRISPRi and CRISPRa techniques are described in greater detail, e.g., in Dominguez et al., Nat. Rev. Mol. Cell Biol. 17(1):5-15 (2016), incorporated herein by reference.

TABLE 1
Exemplary Inhibitory Polynucleotides

| SEQ ID | Type of Interfering | |
|---|---|---|
| NO. | Polynucleotide | Nucleic Acid Sequence |
| 7 | CRISPR gRNA | TGTTGATTGGGTACAATG |
| 8 | CRISPR gRNA | GCAAAAGCCTCCCCTTCG |
| 9 | CRISPR gRNA | GGCATAGCTGCTCGACAG |
| 10 | CRISPR gRNA | CGTAGTCAAATGGCTCTCAC |
| 11 | CRISPR gRNA | CCATTTGACTACGTGGACTG |
| 12 | CRISPR gRNA | CCACAGTCCACGTAGTCAAA |
| 13 | CRISPR gRNA | TGACTACGTGGACTGTGGCC |
| 14 | CRISPR gRNA | GTGAGAGCCATTTGACTACG |
| 15 | CRISPR gRNA | AGAGAATCGACAAAAGGCTC |
| 16 | siRNA (guide strand) | TTTTCCATGAATTTACCCG |
| 17 | siRNA (guide strand) | TTATGCAGGAATGTCAACC |
| 18 | siRNA (guide strand) | ATTACCATAATTCTTGCTG |
| 19 | siRNA (guide strand) | ATTTGAAGAAATGTTCACC |
| 20 | siRNA (guide strand) | TTAAAAAACAATGTGGCCC |
| 21 | siRNA (guide strand) | AAATCAAGAAAGCTTGGTC |
| 22 | siRNA (guide strand) | AAATCTAGAATCTTCACTC |
| 23 | siRNA (guide strand) | ATTAGAAAAATGGTGCTTC |
| 24 | siRNA (guide strand) | TTTAAGAGAAGGCTAGTTC |
| 25 | siRNA (guide strand) | ATTACAAGTACTTTAGAGC |
| 26 | siRNA (guide strand) | AATTTGAAGACCATTAACC |
| 27 | siRNA (guide strand) | TTATTCAATAACCTCCAAC |
| 28 | siRNA (guide strand) | ATATGGAAGAGAATGGGCC |
| 29 | siRNA (guide strand) | ATTATCAGTACTGTCACTG |
| 30 | siRNA (guide strand) | AAATAAATGAGAATGGCCG |
| 31 | siRNA (guide strand) | AATTCCATTATGATGGGAG |
| 32 | siRNA (guide strand) | AAAAAAAAGAGACTCTTCC |
| 33 | siRNA (guide strand) | TATTTGATGAGACTTACAG |
| 34 | siRNA (guide strand) | TTATCAATGAATCTTGCTC |
| 35 | siRNA (guide strand) | ATTACCATCAGTGTCCACC |
| 36 | shRNA (loop bolded) | GAATCTCAGTAGCTCATCTAATCAAGAGTTAGATG |
| | | AGCTACTGAGATTC |
| 37 | shRNA (loop bolded) | GGAAGAGCACAGTAAACTAATTCAAGAGATTAGTT |
| | | TACTGTGCTCTTCC |
| 38 | shRNA (loop bolded) | GACCCCAAGCTCGTGTCTAATTCAAGAGATTAGAC |
| | | ACGAGCTTGGGGTC |
| 39 | shRNA (loop bolded) | GCAGAAGATCTACGACCTATTTCAAGAGAATAGGT |
| | | CGTAGATCTTCTGC |
| 40 | shRNA (loop bolded) | GCAGAACAGCATCAGAGGACTTCAAGAGAGTCCT |
| | | CTGATGCTGTTCTGC |
| 41 | shRNA (loop bolded) | GGGAGTCTTGTAAGCCAGTGATCAAGAGTCACTG |
| | | GCTTACAAGACTCCC |
| 42 | shRNA (loop bolded) | GCCACAGTCCACGTAGTCAAATCAAGAGTTTGACT |
| | | ACGTGGACTGTGGC |
| 43 | shRNA (loop bolded) | GCTCTCACTGGAATCTCTCCTTCAAGAGAGGAGAG |
| | | ATTCCAGTGAGAGC |
| 44 | shRNA (loop bolded) | GGTCCCTAAACAACAGCTCTTTCAAGAGAAGAGCT |
| | | GTTGTTTAGGGACC |
| 45 | shRNA (loop bolded) | GGAAATGACCACCAAGGACAATCAAGAGTTGTCCT |
| | | TGGTGGTCATTTCC |

Small-Molecule Compounds

In some embodiments of the invention, the agent that reduces the level and/or activity of SMARCAL1 in a cell in a subject is a small-molecule compound. Small-molecules compounds include, but are not limited to, small peptides, peptidomimetics (e.g., peptoids), amino acids, amino acid analogs, synthetic polynucleotides, polynucleotide analogs, nucleotides, nucleotide analogs, organic and inorganic compounds (including heterorganic and organometallic compounds) generally having a molecular weight less than about 5,000 grams per mole, e.g., organic or inorganic compounds having a molecular weight less than about 2,000 grams per mole, e.g., organic or inorganic compounds having a molecular weight less than about 1,000 grams per mole, e.g., organic or inorganic compounds having a molecular weight less than about 500 grams per mole, and salts, esters, and other pharmaceutically-acceptable forms of such compounds.

Antibodies

The agent that reduces the level and/or activity of SMARCAL1 in a cell in a subject can be an antibody or antigen-binding fragment thereof. Antibodies and antigen-binding fragments, variants, or derivatives thereof include, but are not limited to, polyclonal, monoclonal, multispecific, human, humanized, primatized, or chimeric antibodies, heteroconjugate antibodies (e.g., bi- tri- and quad-specific antibodies, diabodies, triabodies, and tetrabodies), single-domain antibodies (sdAb), epitope-binding fragments (e.g., Fab, Fab′ and F(ab′)₂), Fd, Fvs, single-chain Fvs (scFv), rlgG, single-chain antibodies, disulfide-linked Fvs (sdFv), fragments including either a VL or VH domain, fragments produced by an Fab expression library, nanobodies, affibodies, aptamers, small-molecule immunopharmaceuticals (SMIPs), and anti-idiotypic (anti-Id) antibodies. Antibody molecules can be of any type (e.g., IgG, IgE, IgM, IgD, IgA, and IgY), class (e.g., IgG1, IgG2, IgG3, IgG4, IgA1 and IgA2) or subclass of immunoglobulin molecule. For example, an agent that reduces the level and/or activity of SMARCAL1 described herein is an antibody (e.g., a polyclonal, monoclonal, humanized, chimeric, or heteroconjugate antibody), or an antigen-binding fragment thereof (e.g., a Fab (e.g., a F(ab′)₂), scFv, SMIP, diabody, a triabody, an affibody, a nanobody, an aptamer, or a single domain antibody) that reduces or blocks the activity and/or function of SMARCAL1 through binding to SMARCAL1.

The making and use of therapeutic antibodies and antigen-binding fragments thereof against a target antigen (e.g., SMARCAL1) is known in the art. Antibodies and antibody fragments can be obtained using conventional techniques known to those of skill in the art, and the fragments can be screened for utility in the same manner as intact antibodies. Antigen-binding fragments can be produced by recombinant DNA techniques, enzymatic or chemical cleavage of intact immunoglobulins, or, in certain cases, by chemical peptide synthesis procedures known in the art. See, for example, the references cited herein above, as well as Zhiqiang An (Editor), Therapeutic Monoclonal Antibodies: From Bench to Clinic. 1st Edition. Wiley 2009, and also Greenfield (Ed.), Antibodies: A Laboratory Manual. (Second edition) Cold Spring Harbor Laboratory Press 2013, for methods of making recombinant antibodies, including antibody engineering, use of degenerate oligonucleotides, 5′-RACE, phage display, and mutagenesis; antibody testing and characterization; antibody pharmacokinetics and pharmacodynamics; antibody purification and storage; and screening and labeling techniques.

### Pharmaceutical Uses

The agents that reduce the level and/or activity of SMARCAL1 in a cell in a subject as described herein are useful in the methods of the invention and, while not bound by theory, are believed to exert their desirable effects through their ability to modulate the level, status, and/or activity of SMARCAL1, e.g., by inhibiting the activity or level of SMARCAL1 in a cell in a mammal.

An aspect of the present invention relates to methods of treating an ALT-positive cancer in a subject in need thereof. In some embodiments, the method includes administering to the subject an effective amount of an agent that reduces the level and/or activity of SMARCAL1 in a cell in the subject.

In some embodiments, the agent that reduces the level and/or activity of SMARCAL1 in a cell in a subject is administered in an amount and for a time effective to result in one (or more, e.g., two or more, three or more, four or more) of: (a) reduced tumor size, (b) reduced rate of tumor growth, (c) increased tumor cell death, (d) reduced tumor progression, (e) reduced number of metastases, (f) reduced rate of metastasis, (g) decreased tumor recurrence, (h) increased survival of subject, and (i) increased progression free survival of a subject.

Another aspect of the present invention relates to methods of treating an ALT-positive cancer having a mutation that results in a loss of function of ATRX in a subject in need thereof. In some embodiments, the method includes administering to the subject an effective amount of an agent that reduces the level and/or activity of SMARCAL1 in a cell in the subject. In some embodiments, the agent that reduces the level and/or activity of SMARCAL1 in a cell in a subject is administered in an amount and for a time effective to result in one (or more, e.g., two or more, three or more, four or more) of: (a) reduced tumor size, (b) reduced rate of tumor growth, (c) increased tumor cell death, (d) reduced tumor progression, (e) reduced number of metastases, (f) reduced rate of metastasis, (g) decreased tumor recurrence, (h) increased survival of subject, and (i) increased progression free survival of a subject. Another aspect of the present invention relates to methods of treating an ALT-positive cancer having a mutation that results in a loss of function of DAXX in a subject in need thereof. In some embodiments, the method includes administering to the subject an effective amount of an agent that reduces the level and/or activity of SMARCAL1 in a cell in the subject. In some embodiments, the agent that reduces the level and/or activity of SMARCAL1 in a cell in a subject is administered in an amount and for a time effective to result in one (or more, e.g., two or more, three or more, four or more) of: (a) reduced tumor size, (b) reduced rate of tumor growth, (c) increased tumor cell death, (d) reduced tumor progression, (e) reduced number of metastases, (f) reduced rate of metastasis, (g) decreased tumor recurrence, (h) increased survival of subject, and (i) increased progression free survival of a subject.

Another aspect of the present invention relates to methods of treating a cancer having a mutation that results in a loss of function of ATRX in a subject in need thereof. In some embodiments, the method includes administering to the subject an effective amount of an agent that reduces the level and/or activity of SMARCAL1 in a cell in the subject. In some embodiments, the agent that reduces the level and/or activity of SMARCAL1 in a cell in a subject is administered in an amount and for a time effective to result in one (or more, e.g., two or more, three or more, four or more) of: (a) reduced tumor size, (b) reduced rate of tumor growth, (c) increased tumor cell death, (d) reduced tumor progression, (e) reduced number of metastases, (f) reduced rate of metastasis, (g) decreased tumor recurrence, (h) increased survival of subject, and (i) increased progression free survival of a subject.

Another aspect of the present invention relates to methods of treating a cancer having a mutation that results in a loss of function of DAXX in a subject in need thereof. In some embodiments, the method includes administering to the subject an effective amount of an agent that reduces the level and/or activity of SMARCAL1 in a cell in the subject. In some embodiments, the agent that reduces the level and/or activity of SMARCAL1 in a cell in a subject is administered in an amount and for a time effective to result in one (or more, e.g., two or more, three or more, four or more) of: (a) reduced tumor size, (b) reduced rate of tumor growth, (c) increased tumor cell death, (d) reduced tumor progression, (e) reduced number of metastases, (f) reduced rate of metastasis, (g) decreased tumor recurrence, (h) increased survival of subject, and (i) increased progression free survival of a subject.

Another aspect of the present invention relates to methods of treating a cancer having a mutation that results in a loss of function of ATRX and DAXX in a subject in need thereof. In some embodiments, the method includes administering to the subject an effective amount of an agent that reduces the level and/or activity of SMARCAL1 in a cell in the subject. In some embodiments, the agent that reduces the level and/or activity of SMARCAL1 in a cell in a subject is administered in an amount and for a time effective to result in one (or more, e.g., two or more, three or more, four or more) of: (a) reduced tumor size, (b) reduced rate of tumor growth, (c) increased tumor cell death, (d) reduced tumor progression, (e) reduced number of metastases, (f) reduced rate of metastasis, (g) decreased tumor recurrence, (h) increased survival of subject, and (i) increased progression free survival of a subject.

Treating cancer can result in a reduction in size or volume of a tumor. For example, after treatment, tumor size is reduced by 5% or greater (e.g., 10%, 20%, 30%, 40%, 50%, 60%, 70%, 80%, 90%, or greater) relative to its size prior to treatment. Size of a tumor may be measured by any reproducible means of measurement. For example, the size of a tumor may be measured as a diameter of the tumor.

Treating cancer may further result in a decrease in number of tumors. For example, after treatment, tumor number is reduced by 5% or greater (e.g., 10%, 20%, 30%, 40%, 50%, 60%, 70%, 80%, 90%, or greater) relative to number prior to treatment. Number of tumors may be measured by any reproducible means of measurement, e.g., the number of tumors may be measured by counting tumors visible to the naked eye or at a specified magnification (e.g., 2×, 3×, 4×, 5×, 10×, or 50×). Treating cancer can result in a decrease in number of metastatic nodules in other tissues or organs distant from the primary tumor site. For example, after treatment, the number of metastatic nodules is reduced by 5% or greater (e.g., 10%, 20%, 30%, 40%, 50%, 60%, 70%, 80%, 90% or greater) relative to number prior to treatment. The number of metastatic nodules may be measured by any reproducible means of measurement. For example, the number of metastatic nodules may be measured by counting metastatic nodules visible to the naked eye or at a specified magnification (e.g., 2×, 10×, or 50×).

Treating cancer can result in an increase in average survival time of a population of subjects treated according to the present invention in comparison to a population of untreated subjects. For example, the average survival time is increased by more than 30 days (more than 60 days, 90 days, or 120 days). An increase in average survival time of a population may be measured by any reproducible means. An increase in average survival time of a population may be measured, for example, by calculating for a population the average length of survival following initiation of treatment with the anti-SMARCAL1 agent described herein. An increase in average survival time of a population may also be measured, for example, by calculating for a population the average length of survival following completion of a first round of treatment with an anti-SMARCAL1 agent described herein.

Treating cancer can also result in a decrease in the mortality rate of a population of treated subjects in comparison to an untreated population. For example, the mortality rate is decreased by more than 2% (e.g., more than 5%, 10%, or 25%). A decrease in the mortality rate of a population of treated subjects may be measured by any reproducible means, for example, by calculating for a population the average number of disease-related deaths per unit time following initiation of treatment with an anti-SMARCAL1 agent described herein. A decrease in the mortality rate of a population may also be measured, for example, by calculating for a population the average number of disease-related deaths per unit time following completion of a first round of treatment with an anti-SMARCAL1 agent as described herein.

Selection of Subjects

Subjects that may be treated using the methods described herein are subjects having an ALT-positive cancer. Subjects that may be treated using the methods described herein are subjects having an ALT-positive cancer having a mutation that results in a loss of function of ATRX and/or DAXX. Subjects that may be treated using methods described herein are subjects having a cancer having a mutation that results in a loss of function of ATRX and/or DAXX.

The types of cancer may include, for example, a soft tissue sarcoma, an osteosarcoma, a pancreatic neuroendocrine tumor (PanNET), a glioma, a pediatric glioblastoma, an astrocytoma, an endometrial cancer, an adrenocortical carcinoma, a neuroepithelial tumor, a non-small cell lung cancer, a bladder cancer, an esophagogastric cancer, a melanoma, a head and neck cancer, a cervical cancer, a Non-Hodgkin lymphoma, a colorectal cancer, a pancreatic cancer, a germ cell tumor, a breast cancer, an ovarian cancer, a hepatobiliary cancer, a renal cell carcinoma, a pheochromocytoma, a prostate cancer, a thyroid cancer, an adrenal gland/peripheral nervous system cancer, a central nervous system cancer, a gall bladder cancer, a hematopoietic neoplasm, a larynx cancer, a liver cancer, an oral cavity cancer, a pleural cancer, a salivary gland carcinoma, a skin cancer, a small intestine cancer, a stomach cancer, a tendon sheath cancer, a testicular cancer, a uterine cancer, or any other type of cancer as described herein.

Adrenal gland/peripheral nervous system cancers include pheochromocytomas, neuroblastomas, and ganglioneuroblastomas. Biliary cancers include extrahepatic cholangiocarcinomas and intrahepatic cholangiocarcinomas. Breast cancers include ductal carcinoma, ductal carcinomas with lobular features, lobular carcinomas, mucinous carcinoma, and tubular carcinomas. Central nervous system cancers include medullary carcinomas, pilocytic astrocytomas, diffuse astrocytomas, anaplastic astrocytomas, glioblastoma multiforme, oligodendrogliomas, anaplastic medulloblastomas, nonanaplastic medulloblastomas, meningiomas, and schwannomas. Colon cancers include adenocarcinomas. Esophageal cancers include adenocarcinomas, squamous cell carcinomas, and small cell carcinomas. Gall bladder cancers include adenocarcinomas. Hematopoietic neoplasms include non-Hodgkin's lymphomas, Hodgkin's lymphomas, and thymomas. Renal cancers include clear cell carcinomas, papillary carcinoma, chromophobe carcinoma, and sarcomatoid carcinomas. Larynx cancers include squamous cell carcinomas. Liver cancers include hepatocellular carcinomas. Lung cancers include adenocarcinoma, squamous cell carcinoma, papillary carcinomas, bronchioloalveolar carcinomas, small cell carcinomas, and large cell carcinomas. PanNET neoplasms include adenocarcinomas, carcinoid tumors, neuroendocrine carcinoid tumors, and paragangliomas. Oral cavity cancers include squamous cell carcinomas. Ovarian cancers include serous carcinomas, clear cell carcinomas, endometriod carcinomas, and mucinous carcinomas. Pleural cancers include malignant mesotheliomas. Prostate cancers include adenocarcinomas and small cell carcinomas. Skin cancers include malignant melanomas, basal cell carcinomas, and squamous cell carcinomas. Small intestine cancers include adenocarcinomas. Soft tissue cancers include gastrointestinal stromal tumors, Kaposi's sarcomas, Ewing's sarcomas, primitive neuroectodermal tumors, undifferentiated pleomorphic sarcomas, fibrosarcomas, leiomyosarcomas, liposarcomas, angiosarcomas, epithelioid sarcomas, malignant peripheral nerve sheath tumors, rhabdomyosarcomas, chondrosarcomas, and neurofibromas. Stomach cancers include adenocarcinomas. Tendon sheath cancers include giant cell tumors. Testicular cancers include seminomas and nonseminomatous germ cell tumors. Thyroid cancers include follicular carcinomas and papillary carcinomas. Uterine cancers include squamous carcinomas, adenocarcinomas, endometrioid carcinomas, serious carcinomas, mixed mesodermal tumors, and clear cell carcinomas.

In some embodiments, the cancer is a soft tissue sarcoma, an osteosarcoma, a PanNET, a glioma, a glioblastoma, a pediatric glioblastoma, an astrocytoma, an endometrial cancer, an adrenocortical carcinoma, a neuroepithelial tumor, a non-small cell lung cancer, a bladder cancer, an esophagogastric cancer, a melanoma, a head and neck cancer, a cervical cancer, a Non-Hodgkin lymphoma, a colorectal cancer, a pancreatic cancer, a germ cell tumor, a breast cancer, an ovarian cancer, a hepatobiliary cancer, a renal cell carcinoma, a pheochromocytoma, a prostate cancer, or a thyroid cancer. In some embodiments, the cancer is a soft tissue sarcoma, an osteosarcoma, a PanNET, or a glioblastoma.

In some embodiments, the cancer is a ganglioneuroblastoma, a diffuse astrocytoma, an anaplastic astrocytoma, a glioblastoma multiforme, an oligodendroglioma, an anaplastic medulloblastoma, a paraganglioma, an undifferentiated pleomorphic sarcoma, a fibrosarcoma, a leiomyosarcoma, a liposarcoma, an angiosargoma, an epithelioid sarcoma, or a nonseminoumatous germ cell tumor.

The cancer may be of early or advanced stage (e.g., a recurrent or metastatic cancer). In some embodiments, the subject has received prior anti-cancer therapy. In some embodiments, the subject has not been previously treated with an anti-cancer therapy. In some embodiments, the cancer is refractory to an anti-cancer therapy (e.g., a telomerase inhibitor as described herein). In some embodiments, the cancer is refractory to targeted therapy with a telomerase inhibitor. In some embodiments, the telomerase inhibitor is a vaccine (e.g., a peptide vaccine) that activates an immune response against telomerase. Peptide vaccines include RIAVAX™ (tertomotide). In other embodiments, the telomerase inhibitor is binds to and inhibits telomerase activity. Inhibitors that bind to and inhibit telomerase activity include the oligonucleotide-lipid conjugate imetelstat.

Combination Therapies

An agent that reduces the level and/or activity of SMARCAL1 in a cell in a subject as described herein, can be administered alone or in combination with an additional anti-cancer therapy. The anti-cancer therapy may be an additional therapeutic agent (e.g., other agents that treat cancer or symptoms associated therewith) or in combination with other types of therapies to treat cancer (e.g., radiological therapies or surgical procedures). In some embodiments, the second therapeutic agent is selected based on tumor type, tumor tissue of origin, tumor stage, or mutation status. In combination treatments, the dosages of one or more of the therapeutic agents may be reduced from standard dosages when administered alone. For example, doses may be determined empirically from drug combinations and permutations or may be deduced by isobolographic analysis (e.g., Black et al., Neurology 65:S3-S6 (2005)). In this case, dosages of the agents or compounds when combined should provide a therapeutic effect.

In some embodiments, the anti-cancer therapy is a telomerase inhibitor. In some embodiments, the telomerase inhibitor is a vaccine (e.g., a peptide vaccine) that activates an immune response against telomerase. Peptide vaccines include RIAVAX™ (tertomotide). In other embodiments, the telomerase inhibitor is binds to and inhibits telomerase activity. Inhibitors that bind to and inhibit telomerase activity include the oligonucleotide-lipid conjugate imetelstat.

In other embodiments, the anti-cancer therapy is a checkpoint inhibitor. In some embodiments, the inhibitor of checkpoint is an inhibitory antibody (e.g., a monospecific antibody, such as a monoclonal antibody). The antibody may be humanized or fully human. In some embodiments, the inhibitor of checkpoint is a fusion protein, e.g., an Fc-receptor fusion protein. In some embodiments, the inhibitor of checkpoint is an agent, such as an antibody, that interacts with a checkpoint protein. In some embodiments, the inhibitor of checkpoint is an agent, such as an antibody, that interacts with the ligand of a checkpoint protein. In some embodiments, the inhibitor of checkpoint is an inhibitor (e.g., an inhibitory antibody or small-molecule inhibitor) of CTLA-4 (e.g., an anti-CTLA4 antibody or a fusion protein, such as ipilimumab/YERVOY® or tremelimumab). In some embodiments, the inhibitor of checkpoint is an inhibitor (e.g., an inhibitory antibody or small-molecule inhibitor) of PD-1 (e.g., nivolumab/OPDIVO®; pembrolizumab/KEYTRUDA®; cemiplimab/LIBTAYO®; or pidilizumab/CT-011). In some embodiments, the inhibitor of checkpoint is an inhibitor (e.g., an inhibitory antibody or small-molecule inhibitor) of PDL1 (e.g., MPDL3280A/RG7446/atezolizumab; MED14736/durvalumab; MSB0010718C/avelumab;). In some embodiments, the inhibitor of checkpoint is an inhibitor (e.g., an inhibitory antibody or Fc fusion or small-molecule inhibitor) of PDL2 (e.g., a PDL2/Ig fusion protein, such as AMP 224). In some embodiments, the inhibitor of checkpoint is an inhibitor (e.g., an inhibitory antibody or small-molecule inhibitor) of B7-H3 (e.g., MGA271), B7-H4, BTLA, HVEM, TIM3, GAL9, LAG3, VISTA, KIR, 2B4, CD160, CGEN-15049, CHK 1, CHK2, A2aR, B-7 family ligands, or a combination thereof.

In some embodiments, the anti-cancer therapy is a biologic, such as a cytokine (e.g., interferon or an interleukin (e.g., IL-2)) used in cancer treatment. In some embodiments the biologic is an anti-angiogenic agent, such as an anti-VEGF agent, e.g., bevacizumab (AVASTIN®). In some embodiments the biologic is an immunoglobulin-based biologic, e.g., a monoclonal antibody (e.g., a humanized antibody, a fully human antibody, an Fc fusion protein or a functional fragment thereof) that agonizes a target to stimulate an anti-cancer response, or antagonizes an antigen important for cancer. Such agents include RITUXAN® (Rituximab); ZENAPAX® (Daclizumab); SIMULECT® (Basiliximab); SYNAGIS® (Palivizumab); REMICADE® (Infliximab); HERCEPTIN® (Trastuzumab); MYLOTARG™ (Gemtuzumab ozogamicin); CAMPATH® (Alemtuzumab); ZEVALIN® (Ibritumomab tiuxetan); HUMIRA® (Adalimumab); XOLAIR® (Omalizumab); BEXXAR® (Tositumomab-I-131); RAPTIVA® (Efalizumab); ERBITUX® (Cetuximab); AVASTIN® (Bevacizumab); TYSABRI® (Natalizumab); ACTEMRA® (Tocilizumab); VECTIBIX® (Panitumumab); LUCENTIS® (Ranibizumab); SOLIRIS® (Eculizumab); CIMZIA® (Certolizumab pegol); SIMPONI® (Golimumab); ILARIS® (Canakinumab); STELARA® (Ustekinumab); ARZERRA® (Ofatumumab); PROLIA® (Denosumab); Numax (Motavizumab); ABThrax (Raxibacumab); BENLYSTA® (Belimumab); YERVOY® (Ipilimumab); ADCETRIS® (Brentuximab Vedotin); PERJETA® (Pertuzumab); KADCYLA® (Ado-trastuzumab emtansine); and GAZYVA® (Obinutuzumab). Also included are antibody-drug conjugates.

In some embodiments, the anti-cancer therapy is a chemotherapeutic agent (e.g., a cytotoxic agent or other chemical compound useful in the treatment of cancer). These include alkylating agents, antimetabolites, folic acid analogs, pyrimidine analogs, purine analogs and related inhibitors, vinca alkaloids, epipodopyyllotoxins, antibiotics, L-Asparaginase, topoisomerase inhibitors, interferons, platinum coordination complexes, anthracenedione substituted urea, methyl hydrazine derivatives, adrenocortical suppressant, adrenocorticosteroides, progestins, estrogens, antiestrogen, androgens, antiandrogen, and gonadotropin-releasing hormone analog. Also included is 5-fluorouracil (5-FU), leucovorin, irenotecan, oxaliplatin, capecitabine, paclitaxel, and doxetaxel. Non-limiting examples of chemotherapeutic agents include alkylating agents, such as thiotepa and cyclosphosphamide; alkyl sulfonates, such as busulfan, improsulfan and piposulfan; aziridines, such as benzodopa, carboquone, meturedopa, and uredopa; ethylenimines and methylamelamines including altretamine, triethylenemelamine, trietylenephosphoramide, triethiylenethiophosphoramide and trimethylolomelamine; acetogenins (especially bullatacin and bullatacinone); a camptothecin (including the synthetic analogue topotecan); bryostatin; callystatin; CC-1065 (including its adozelesin, carzelesin and bizelesin synthetic analogues); cryptophycins (particularly cryptophycin 1 and cryptophycin 8); dolastatin; duocarmycin (including the synthetic analogues, KW-2189 and CB1-TM1); eleutherobin; pancratistatin; a sarcodictyin; spongistatin; nitrogen mustards, such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas, such as carmustine, chlorozotocin, fotemustine, lomustine, nimustine, and ranimnustine; antibiotics, such as the enediyne antibiotics (e.g., calicheamicin, especially calicheamicin gammall and calicheamicin omegall (see, e.g., Agnew, Chem. Intl. Ed Engl. 33:183-186 (1994)); dynemicin, including dynemicin A; bisphosphonates, such as clodronate; an esperamicin; as well as neocarzinostatin chromophore and related chromoprotein enediyne antiobiotic chromophores), aclacinomysins, actinomycin, authramycin, azaserine, bleomycins, cactinomycin, carabicin, caminomycin, carzinophilin, chromomycinis, dactinomycin, daunorubicin, detorubicin, 6-diazo-5-oxo-L-norleucine, ADRIAMYCIN® (doxorubicin, including morpholino-doxorubicin, cyanomorpholino-doxorubicin, 2-pyrrolino-doxorubicin and deoxydoxorubicin), epirubicin, esorubicin, idarubicin, marcellomycin, mitomycins, such as mitomycin C, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites, such as methotrexate and 5-fluorouracil; folic acid analogues, such as denopterin, pteropterin, trimetrexate; purine analogs, such as fludarabine, 6-mercaptopurine, thiamiprine, thioguanine; pyrimidine analogs, such as ancitabine, azacitidine, 6-azauridine, carmofur, cytarabine, dideoxyuridine, doxifluridine, enocitabine, floxuridine; androgens, such as calusterone, dromostanolone propionate, epitiostanol, mepitiostane, testolactone; anti-adrenals, such as aminoglutethimide, mitotane, trilostane; folic acid replenisher, such as frolinic acid; aceglatone; aldophosphamide glycoside; aminolevulinic acid; eniluracil; amsacrine; bestrabucil; bisantrene; edatraxate; defofamine; demecolcine; diaziquone; elfomithine; elliptinium acetate; an epothilone; etoglucid; gallium nitrate; hydroxyurea; lentinan; lonidainine; maytansinoids, such as maytansine and ansamitocins; mitoguazone; mitoxantrone; mopidanmol; nitraerine; pentostatin; phenamet; pirarubicin; losoxantrone; podophyllinic acid; 2-ethylhydrazide; procarbazine; PSK® polysaccharide complex (JHS Natural Products, Eugene, OR); razoxane; rhizoxin; sizofuran; spirogermanium; tenuazonic acid; triaziquone; 2,2′,2″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside; cyclophosphamide; thiotepa; taxoids, e.g., TAXOL® (paclitaxel; Bristol-Myers Squibb Oncology, Princeton, N.J.), ABRAXANE®, cremophor-free, albumin-engineered nanoparticle formulation of paclitaxel (American Pharmaceutical Partners, Schaumberg, Ill.), and TAXOTERE® doxetaxel (Rhone-Poulenc Rorer, Antony, France); chloranbucil; GEMZAR® gemcitabine; 6-thioguanine; mercaptopurine; platinum coordination complexes, such as cisplatin, oxaliplatin and carboplatin; vinblastine; platinum; etoposide (VP-16); ifosfamide; mitoxantrone; vincristine; NAVELBINE® vinorelbine; novantrone; teniposide; edatrexate; daunomycin; aminopterin; XELODA®; ibandronate; irinotecan (e.g., CPT-11); topoisomerase inhibitor RFS 2000; difluoromethylornithine (DMFO); retinoids, such as retinoic acid; capecitabine; and pharmaceutically-acceptable salts, acids or derivatives of any of the above. Two or more chemotherapeutic agents can be used in a cocktail to be administered in combination with the first therapeutic agent described herein. Suitable dosing regimens of combination chemotherapies are known in the art and described in, for example, Saltz et al., Proc. Am. Soc. Clin. Oncol. 18:233a (1999), and Douillard et al., Lancet 355(9209):1041-1047 (2000).

In some embodiments, the anti-cancer therapy is a T cell adoptive transfer therapy. In some embodiments, the T cell is an activated T cell. The T cell may be modified to express a chimeric antigen receptor (CAR). CAR modified T (CAR-T) cells can be generated by any method known in the art. For example, the CAR-T cells can be generated by introducing a suitable expression vector encoding the CAR to a T cell. Prior to expansion and genetic modification of the T cells, a source of T cells is obtained from a subject. T cells can be obtained from a number of sources, including peripheral blood mononuclear cells, bone marrow, lymph node tissue, cord blood, thymus tissue, tissue from a site of infection, ascites, pleural effusion, spleen tissue, and tumors. In certain embodiments of the present invention, any number of T cell lines available in the art, may be used. In some embodiments, the T cell is an autologous T cell. Whether prior to or after genetic modification of the T cells to express a desirable protein (e.g., a CAR), the T cells can be activated and expanded generally using methods as described, for example, in U.S. Pat. Nos. 6,352,694; 6,534,055; 6,905,680; 6,692,964; 5,858,358; 6,887,466; 6,905,681; 7,144,575; 7,067,318; 7,172,869; 7,232,566; 7,175,843; 5,883,223; 6,905,874; 6,797,514; 6,867,041; and U.S. Patent Application Publication No. 20060121005.

The additional anti-cancer therapy may be a non-drug treatment. For example, the additional therapeutic agent is radiation therapy, cryotherapy, hyperthermia, and/or surgical excision of tumor tissue.

In any of the combination embodiments described herein, the agent that reduces the level and/or activity of SMARCAL1 in a cell in a subject and additional therapeutic agents are administered simultaneously or sequentially, in either order. The agent that reduces the level and/or activity of SMARCAL1 in a cell in a subject may be administered immediately, up to 1 hour, up to 2 hours, up to 3 hours, up to 4 hours, up to 5 hours, up to 6 hours, up to 7 hours, up to 8 hours, up to 9 hours, up to 10 hours, up to 11 hours, up to 12 hours, up to 13 hours, up to 14 hours, up to15 hours, up to 16 hours, up to 17 hours, up 18 hours, up to 19 hours, up to 20 hours, up to 21 hours, up to 22 hours, up to 23 hours, up to 24 hours, or up to 1-7, 1-14, 1-21, or 1-30 days before or after the additional therapeutic agent (e.g., an anti-cancer therapy).

Delivery of Anti-SMARCAL1 Agents

The delivery of anti-SMARCAL1 agents to a subject (e.g., to a cell within a subject) can be achieved in a number of different ways. For example, delivery may be performed by contacting a cell with an anti-SMARCAL1 agent either in vitro or in vivo. In vivo delivery may also be performed directly by administering a composition comprising an agent that reduces the level and/or activity of SMARCAL1 in a cell in a subject, to a subject. Alternatively, in vivo delivery may be performed indirectly by administering one or more vectors that encode and direct the expression of the anti-SMARCAL1 agent. These alternatives are discussed further below.

In general, any method of delivering a nucleic acid molecule (in vitro or in vivo) can be adapted for use with an anti-SMARCAL1 agent of the invention (see e.g., Akhtar S. and Julian R L., (1992) Trends Cell. Biol. 2(5):139-144 and W094/02595, which are incorporated herein by reference). For in vivo delivery, factors to consider in order to deliver an anti-SMARCAL1 agent include, for example, biological stability of the delivered molecule, prevention of non-specific effects, and accumulation of the delivered molecule in the target tissue. The non-specific effects of an anti-SMARCAL1 agent can be minimized by local administration, for example, by direct injection or implantation into a tissue or topically administering the preparation. Local administration to a treatment site maximizes local concentration of the agent, limits the exposure of the agent to systemic tissues that can otherwise be harmed by the agent or that can degrade the agent, and permits a lower total dose of the anti-SMARCAL1 agent to be administered.

For administering an anti-SMARCAL1 agent systemically for the treatment of a disease, the polynucleotide agent can be modified or alternatively delivered using a drug delivery system; both methods act to prevent the rapid degradation of the polynucleotide by endo- and exo-nucleases in vivo. Modification of the polynucleotide or the pharmaceutical carrier can also permit targeting of the anti-SMARCAL1 agent to the target tissue and avoid undesirable off-target effects. Anti-SMARCAL1 agents can be modified by chemical conjugation to lipophilic groups such as cholesterol to enhance cellular uptake and prevent degradation. In an alternative embodiment, the anti-SMARCAL1 agent can be delivered using drug delivery systems such as a nanoparticle, a dendrimer, a polymer, liposomes, or a cationic delivery system. Positively-charged cationic delivery systems facilitate binding of a polynucleotide (negatively-charged) and also enhance interactions at the negatively-charged cell membrane to permit efficient uptake of a polynucleotide by the cell. Cationic lipids, dendrimers, or polymers can either be bound to an anti-SMARCAL1 agent, or induced to form a vesicle or micelle (see e.g., Kim S H. et al., (2008) Journal of Controlled Release 129(2):107-116) that encases an anti-SMARCAL1 agent. The formation of vesicles or micelles further prevents degradation of the anti-SMARCAL1 agent when administered systemically. Methods for making and administering cationic-polynucleotide complexes are well within the abilities of one skilled in the art.

Vector Delivery Methods

The agent that reduces the level and/or activity of SMARCAL1 in a cell in a subject can be expressed from transcription units inserted into DNA or RNA vectors (see, e.g., Couture, A, et al., TIG. (1996), 12:5-10; Skillern, A., et al., International PCT Publication No. WO 00/22113, Conrad, International PCT Publication No. WO 00/22114, and Conrad, U.S. Pat. No. 6,054,299). Expression can be transient (on the order of hours to weeks) or sustained (weeks to months or longer), depending upon the specific construct used and the target tissue or cell type. These transgenes can be introduced as a linear construct, a circular plasmid, or a viral vector, which can be an integrating or non-integrating vector. The transgene can also be constructed to permit it to be inherited as an extrachromosomal plasmid (Gassmann, et al., (1995) Proc. Natl. Acad. Sci. USA 92:1292).

The individual strand or strands of an anti-SMARCAL1 polynucleotide can be transcribed from a promoter on an expression vector. Where two separate strands are to be expressed to generate, for example, an anti-SMARCAL1 polynucleotide, two separate expression vectors can be co-introduced (e.g., by transfection or infection) into a target cell. Alternatively each individual strand of an anti-SMARCAL1 polynucleotide can be transcribed by promoters both of which are located on the same expression plasmid. In one embodiment, an anti-SMARCAL1 agent is expressed as inverted repeat polynucleotides joined by a linker polynucleotide sequence such that the polynucleotide has a stem-and-loop structure.

Polynucleotide expression vectors are generally DNA plasmids or viral vectors. Expression vectors compatible with eukaryotic cells, preferably those compatible with vertebrate cells, can be used to produce recombinant constructs for the expression of an anti-SMARCAL1 agent as described herein. Eukaryotic cell expression vectors are well known in the art and are available from a number of commercial sources. Typically, such vectors are provided containing convenient restriction sites for insertion of the desired nucleic acid segment. Delivery of anti-SMARCAL1 expressing vectors can be systemic, such as by intravenous or intramuscular administration, by administration to target cells ex-planted from the patient followed by reintroduction into the patient, or by any other means that allows for introduction into a desired target cell.

In some embodiments, the agent that reduces the level and/or activity of SMARCAL1 in a cell in a subject is delivered by a viral vector (e.g., a viral vector expressing an anti-SMARCAL1 agent, such as a polynucleotide as described herein). Viral genomes provide a rich source of vectors that can be used for the efficient delivery of exogenous genes into a mammalian cell. Viral genomes are particularly useful vectors for gene delivery because the polynucleotides contained within such genomes are typically incorporated into the nuclear genome of a mammalian cell by generalized or specialized transduction. These processes occur as part of the natural viral replication cycle, and do not require added proteins or reagents in order to induce gene integration. Examples of viral vectors include a retrovirus (e.g., Retroviridae family viral vector), adenovirus (e.g., Ad5, Ad26, Ad34, Ad35, and Ad48), parvovirus (e.g., adeno-associated viruses), coronavirus, negative-strand RNA viruses, such as orthomyxovirus (e.g., influenza virus), rhabdovirus (e.g., rabies and vesicular stomatitis virus), paramyxovirus (e.g., measles and Sendai), positive-strand RNA viruses, such as picornavirus and alphavirus, and double-stranded DNA viruses including adenovirus, herpesvirus (e.g., Herpes Simplex virus types 1 and 2, Epstein-Barr virus, cytomegalovirus, replication deficient herpes virus), and poxvirus (e.g., vaccinia, modified vaccinia Ankara, fowlpox and canarypox). Other viruses include Norwalk virus, togavirus, flavivirus, reoviruses, papovavirus, hepadnavirus, human papilloma virus, human foamy virus, and hepatitis virus, for example. Examples of retroviruses include: avian leukosis-sarcoma, avian C-type viruses, mammalian C-type, B-type viruses, D-type viruses, oncoretroviruses, HTLV-BLV group, lentivirus, alpharetrovirus, gammaretrovirus, spumavirus (Coffin, J. M., Retroviridae: The viruses and their replication, Virology (Third Edition) Lippincott-Raven, Philadelphia, 1996). Other examples include murine leukemia viruses, murine sarcoma viruses, mouse mammary tumor virus, bovine leukemia virus, feline leukemia virus, feline sarcoma virus, avian leukemia virus, human T cell leukemia virus, baboon endogenous virus, Gibbon ape leukemia virus, Mason Pfizer monkey virus, simian immunodeficiency virus, simian sarcoma virus, Rous sarcoma virus and lentiviruses. Other examples of vectors are described, for example, in U.S. Pat. No. 5,801,030, the teachings of which are incorporated herein by reference.

Viral vectors include lentiviral vectors, AAVs, and retroviral vectors. Lentiviral vectors and AAVs can integrate into the genome without cell divisions, and both types have been tested in pre-clinical animal studies. Methods for preparation of AAVs are described in the art e.g., in U.S. Pat. Nos. 5,677,158, 6,309,634, and 6,683,058, each of which is incorporated herein by reference. Methods for preparation and in vivo administration of lentiviruses are described in US 20020037281 (incorporated herein by reference). Preferably, a lentiviral vector is a replication-defective lentivirus particle. Such a lentivirus particle can be produced from a lentiviral vector comprising a 5′ lentiviral LTR, a tRNA binding site, a packaging signal, a promoter operably linked to a polynucleotide signal encoding the fusion protein, an origin of second strand DNA synthesis and a 3′ lentiviral LTR.

Retroviruses are most commonly used in human clinical trials, as they carry 7-8 kb, and have the ability to infect cells and have their genetic material stably integrated into the host cell with high efficiency (see, e.g., WO 95/30761; WO 95/24929, each of which is incorporated herein by reference). Preferably, a retroviral vector is replication defective. This prevents further generation of infectious retroviral particles in the target tissue. Thus, the replication defective virus becomes a “captive” transgene stable incorporated into the target cell genome. This is typically accomplished by deleting the gag, env, and pol genes (along with most of the rest of the viral genome). Heterologous nucleic acids are inserted in place of the deleted viral genes. The heterologous genes may be under the control of the endogenous heterologous promoter, another heterologous promoter active in the target cell, or the retroviral 5′ LTR (the viral LTR is active in diverse tissues).

These delivery vectors described herein can be made target-specific by attaching, for example, a sugar, a glycolipid, or a protein (e.g., an antibody to a target cell receptor).

Reversible delivery expression systems may also be used. The Cre-loxP or FLP/FRT system and other similar systems can be used for reversible delivery-expression of one or more of the above-described nucleic acids. See WO2005/112620, WO2005/039643, US20050130919, US20030022375, US20020022018, US20030027335, and US20040216178. In particular, the reversible delivery-expression system described in US20100284990 can be used to provide a selective or emergency shut-off.

Membranous Molecular Delivery Methods

Several membranous molecular delivery methods exist for delivery of anti-SMARCAL1 agents including polymeric, biodegradable microparticle, or microcapsule delivery devices known in the art. For example, a colloidal dispersion system may be used for targeted delivery an anti-SMARCAL1 agent described herein. Colloidal dispersion systems include macromolecule complexes, nanocapsules, microspheres, beads, and lipid-based systems including oil-in-water emulsions, micelles, mixed micelles, and liposomes. Liposomes are artificial membrane vesicles that are useful as delivery vehicles in vitro and in vivo. It has been shown that large unilamellar vesicles, which range in size from 0.2-4.0 μm can encapsulate a substantial percentage of an aqueous buffer containing large macromolecules. Liposomes are useful for the transfer and delivery of active ingredients to the site of action.

Because the liposomal membrane is structurally similar to biological membranes, when liposomes are applied to a tissue, the liposomal bilayer fuses with bilayer of the cellular membranes. In one example, as the merging of the liposome and cell progresses, the internal aqueous contents that include the anti-SMARCAL1 agent are delivered into the cell where the polynucleotide can specifically bind to a target RNA and can mediate RNAi. In some cases the liposomes are also specifically targeted, e.g., to direct the anti-SMARCAL1 agent to particular cell types. The composition of the liposome is usually a combination of phospholipids, usually in combination with steroids, especially cholesterol. Other phospholipids or other lipids may also be used. The physical characteristics of liposomes depend on pH, ionic strength, and the presence of divalent cations. A liposome containing an anti-SMARCAL1 agent can be prepared by a variety of methods that are well known in the art.

Liposomes fall into two broad classes. Cationic liposomes are positively-charged liposomes which interact with the negatively-charged nucleic acid molecules to form a stable complex. The positively-charged nucleic acid/liposome complex binds to the negatively-charged cell surface and is internalized in an endosome. Due to the acidic pH within the endosome, the liposomes are ruptured, releasing their contents into the cell cytoplasm (Wang et al. (1987) Biochem. Biophys. Res. Commun., 147:980-985).

Liposomes, which are pH-sensitive or negatively-charged, entrap nucleic acids rather than complex with them. Since both the nucleic acid and the lipid are similarly charged, repulsion rather than complex formation occurs. Nevertheless, some nucleic acid is entrapped within the aqueous interior of these liposomes. pH-sensitive liposomes have been used to deliver nucleic acids encoding the thymidine kinase gene to cell monolayers in culture. Expression of the exogenous gene was detected in the target cells (Zhou et al. (1992) Journal of Controlled Release, 19:269-274).

One major type of liposomal composition includes phospholipids other than naturally-derived phosphatidylcholine. Neutral liposome compositions, for example, can be formed from dimyristoyl phosphatidylcholine (DMPC) or dipalmitoyl phosphatidylcholine (DPPC). Anionic liposome compositions generally are formed from dimyristoyl phosphatidylglycerol, while anionic fusogenic liposomes are formed primarily from dioleoyl phosphatidylethanolamine (DOPE). Another type of liposomal composition is formed from phosphatidylcholine (PC) such as, for example, soybean PC, and egg PC. Another type is formed from mixtures of phospholipid and/or phosphatidylcholine and/or cholesterol.

Liposomes may also be sterically-stabilized liposomes, comprising one or more specialized lipids that result in enhanced circulation lifetimes relative to liposomes lacking such specialized lipids. Examples of sterically-stabilized liposomes are those in which part of the vesicle-forming lipid portion of the liposome (A) comprises one or more glycolipids, such as monosialoganglioside G_{M1}, or (B) is derivatized with one or more hydrophilic polymers, such as a polyethylene glycol (PEG) moiety. While not wishing to be bound by any particular theory, it is thought in the art that, at least for sterically-stabilized liposomes containing gangliosides, sphingomyelin, or PEG-derivatized lipids, the enhanced circulation half-life of these sterically-stabilized liposomes derives from a reduced uptake into cells of the reticuloendothelial system (RES) (Allen et al., (1987) FEBS Letters, 223:42; Wu et al., (1993) Cancer Research, 53:3765).

Further advantages of liposomes include: liposomes obtained from natural phospholipids are biocompatible and biodegradable; liposomes can incorporate a wide range of water and lipid soluble drugs; liposomes can protect encapsulated polynucleotides in their internal compartments from metabolism and degradation (Rosoff, in “Pharmaceutical Dosage Forms,” Lieberman, Rieger and Banker (Eds.), 1988, volume 1, p. 245). Important considerations in the preparation of liposome formulations are the lipid surface charge, vesicle size and the aqueous volume of the liposomes.

The targeting of liposomes is also possible based on, for example, organ-specificity, cell-specificity, and organelle-specificity and is known in the art. In the case of a liposomal targeted delivery system, lipid groups can be incorporated into the lipid bilayer of the liposome in order to maintain the targeting ligand in stable association with the liposomal bilayer. Various linking groups can be used for joining the lipid chains to the targeting ligand. Additional methods are known in the art and are described, for example in U.S. Patent Application Publication No. 20060058255, the linking groups of which are herein incorporated by reference.

Liposomes that include polynucleotides can be made highly deformable. Such deformability can enable the liposomes to penetrate through pores that are smaller than the average radius of the liposome. For example, transfersomes are a type of deformable liposomes. Transferosomes can be made by adding surface edge activators, usually surfactants, to a standard liposomal composition. Due to the lipid properties, these transferosomes can be self-optimizing (adaptive to the shape of pores, e.g., in the skin), self-repairing, can frequently reach their targets without fragmenting, and are often self-loading.

The anti-SMARCAL1 agents for use in the methods of the invention can also be provided as micellar formulations. Micelles are a particular type of molecular assembly in which amphipathic molecules are arranged in a spherical structure such that all the hydrophobic portions of the molecules are directed inward, leaving the hydrophilic portions in contact with the surrounding aqueous phase. The converse arrangement exists if the environment is hydrophobic.

Lipid Nanoparticle-Based Delivery Methods

Anti-SMARCAL1 agents may be fully encapsulated in a lipid formulation, e.g., a lipid nanoparticle (LNP), or other nucleic acid-lipid particle. LNPs are extremely useful for systemic applications, as they exhibit extended circulation lifetimes following intravenous (i.v.) injection and accumulate at distal sites (e.g., sites physically separated from the administration site). LNPs include “pSPLP,” which include an encapsulated condensing agent-nucleic acid complex as set forth in PCT Publication No. WO 00/03683. The particles may have a mean diameter of about 50 nm to about 150 nm, more typically about 60 nm to about 130 nm, more typically about 70 nm to about 110 nm, most typically about 70 nm to about 90 nm, and are substantially nontoxic. In addition, the nucleic acids when present in the nucleic acid-lipid particles are resistant in aqueous solution to degradation with a nuclease. Nucleic acid-lipid particles and their method of preparation are disclosed in, e.g., U.S. Pat. Nos. 5,976,567; 5,981,501; 6,534,484; 6,586,410; 6,815,432; U.S. Publication No. 2010/0324120 and PCT Publication No. WO 96/40964.

In one embodiment, the lipid to anti-SMARCAL1 agent (mass/mass ratio) (e.g., lipid to polynucleotide ratio) will be in the range of from about 1:1 to about 50:1, from about 1:1 to about 25:1, from about 3:1 to about 15:1, from about 4:1 to about 10:1, from about 5:1 to about 9:1, or about 6:1 to about 9:1. Ranges intermediate to the above recited ranges are also contemplated to be part of the invention.

Non-limiting examples of cationic lipid include N,N-dioleyl-N,N-dimethylammonium chloride (DODAC), N,N-distearyl-N,N-dimethylammonium bromide (DDAB), N-(I-(2,3-dioleoyloxy)propyl)-N,N,N-trimethylammonium chloride (DOTAP), N-(I-(2,3-dioleyloxy)propyl)-N,N,N-trimethylammonium chloride (DOTMA), N,N-dimethyl-2,3-dioleyloxy)propylamine (DODMA), 1,2-DiLinoleyloxy-N,N-dimethylaminopropane (DLinDMA), 1,2-Dilinolenyloxy-N,N-dimethylaminopropane (DLenDMA), 1,2-Dilinoleylcarbamoyloxy-3-dimethylaminopropane (DLin-C-DAP), 1,2-Dilinoleyoxy-3-(dimethylamino)acetoxypropane (DLin-DAC), 1,2-Dilinoleyoxy-3-morpholinopropane (DLin-MA), 1,2-Dilinoleoyl-3-dimethylaminopropane (DLinDAP), 1,2-Dilinoleylthio-3-dimethylaminopropane (DLin-S-DMA), 1-Linoleoyl-2-linoleyloxy-3-dimethylaminopropane (DLin-2-DMAP), 1,2-Dilinoleyloxy-3-trimethylaminopropane chloride salt (DLin-TMA.CI), 1,2-Dilinoleoyl-3-trimethylaminopropane chloride salt (DLin-TAP.CI), 1,2-Dilinoleyloxy-3-(N-methylpiperazino)propane (DLin-MPZ), or 3-(N,N-Dilinoleylamino)-1,2-propanediol (DLinAP), 3-(N,N-Dioleylamino)-1,2-propanedio (DOAP), 1,2-Dilinoleyloxo-3-(2-N,N-dimethylamino)ethoxypropane (DLin-EG-DMA), 1,2-Dilinolenyloxy-N,N-dimethylaminopropane (DLinDMA), 2,2-Dilinoleyl-4-dimethylaminomethyl-[1,3]-dioxolane (DLin-K-DMA) or analogs thereof, (3aR,5s,6aS)-N,N-dimethyl-2,2-di((9Z,12Z)-octadeca-9,12-dienyetetrahydro-- 3aH-cyclopenta[d][1,3]dioxol-5-amine (ALN100), (6Z,9Z,28Z,31Z)-heptatriaconta-6,9,28,31-tetraen-19-yl4-(dimethylamino)bu-tanoate (MC3), 1,1′-(2-(4-(2-((2-(bis(2-hydroxydodecyl)amino)ethyl)(2-hydroxydodecyl)ami-no)ethyl)piperazin-1-yeethylazanediyedidodecan-2-ol (Tech G1), or a mixture thereof. The cationic lipid can comprise, for example, from about 20 mol % to about 50 mol % or about 40 mol % of the total lipid present in the particle.

The ionizable/non-cationic lipid can be an anionic lipid or a neutral lipid including, but not limited to, distearoylphosphatidylcholine (DSPC), dioleoylphosphatidylcholine (DOPC), dipalmitoylphosphatidylcholine (DPPC), dioleoylphosphatidylglycerol (DOPG), dipalmitoylphosphatidylglycerol (DPPG), dioleoyl-phosphatidylethanolamine (DOPE), palmitoyloleoylphosphatidylcholine (POPC), palmitoyloleoylphosphatidylethanolamine (POPE), dioleoyl-phosphatidylethanolamine 4-(N-maleimidomethyl)-cyclohexane-1-carboxylate (DOPE-mal), dipalmitoyl phosphatidyl ethanolamine (DPPE), dimyristoylphosphoethanolamine (DMPE), distearoyl-phosphatidyl-ethanolamine (DSPE), 16-O-monomethyl PE, 16-O-dimethyl PE, 18-1-trans PE, 1-stearoyl-2-oleoyl-phosphatidyethanolamine (SOPE), cholesterol, or a mixture thereof. The non-cationic lipid can be, for example, from about 5 mol % to about 90 mol %, about 10 mol %, or about 58 mol % if cholesterol is included, of the total lipid present in the particle.

The conjugated lipid that inhibits aggregation of particles can be, for example, a polyethyleneglycol (PEG)-lipid including, without limitation, a PEG-diacylglycerol (DAG), a PEG-dialkyloxypropyl (DAA), a PEG-phospholipid, a PEG-ceramide (Cer), or a mixture thereof. The PEG-DAA conjugate can be, for example, a PEG-dilauryloxypropyl (Ci₂), a PEG-dimyristyloxypropyl (Ci₄), a PEG-dipalmityloxypropyl (Ci₆), or a PEG-distearyloxypropyl (C]₈). The conjugated lipid that prevents aggregation of particles can be, for example, from 0 mol % to about 20 mol % or about 2 mol % of the total lipid present in the particle.

In some embodiments, the nucleic acid-lipid particle further includes cholesterol at, e.g., about 10 mol % to about 60 mol % or about 48 mol % of the total lipid present in the particle.

Pharmaceutical Compositions and Routes of Administration

Anti-SMARCAL1 agents for use in the methods described herein may be placed into a pharmaceutically-acceptable suspension, solution, or emulsion.

The anti-SMARCAL1 agents described herein may be administered, for example, by parenteral, intratumoral, oral, buccal, sublingual, nasal, rectal, patch, pump, or transdermal administration.

Parenteral administration includes intravenous, intraperitoneal, subcutaneous, intramuscular, transepithelial, nasal, intrapulmonary, intrathecal, rectal, and topical modes of administration. Parenteral administration may be by continuous infusion over a selected period of time.

In some embodiments, an anti-SMARCAL1 agent for use in the methods described herein is administered intratumorally, for example, as an intratumoral injection. Intratumoral injection is injection directly into the tumor vasculature and is specifically contemplated for discrete, solid, accessible tumors. Local, regional, or systemic administration also may be appropriate. An anti-SMARCAL1 agent described herein may advantageously be contacted by administering an injection or multiple injections to the tumor, spaced for example, at approximately, 1 cm intervals. In the case of surgical intervention, anti-SMARCAL1 agents may be used preoperatively, such as to render an inoperable tumor subject to resection. Continuous administration also may be applied where appropriate, for example, by implanting a catheter into a tumor or into tumor vasculature.

In some embodiments, an anti-SMARCAL1 agent described herein is administered parenterally (e.g., intravenously). Solutions of an anti-SMARCAL1 agent described herein can be prepared in water suitably mixed with a surfactant, such as hydroxypropylcellulose. Dispersions can also be prepared in glycerol, liquid polyethylene glycols, DMSO, and mixtures thereof with or without alcohol, and in oils. Under ordinary conditions of storage and use, these preparations may contain a preservative to prevent the growth of microorganisms. Conventional procedures and ingredients for the selection and preparation of suitable formulations are described, for example, in Remington's Pharmaceutical Sciences (2012, 22^{nd} ed.) and in The United States Pharmacopeia: The National Formulary (USP 41 NF36), published in 2018. The pharmaceutical forms suitable for injectable use include sterile aqueous solutions or dispersions and sterile powders for the extemporaneous preparation of sterile injectable solutions or dispersions. In all cases the form must be sterile and must be fluid to the extent that may be easily administered via syringe.

An anti-SMARCAL1 agent described herein may be orally administered, for example, with an inert diluent or with an assimilable edible carrier, may be enclosed in hard or soft shell gelatin capsules, may be compressed into tablets, or may be incorporated directly with the food of the diet. For oral therapeutic administration, an anti-SMARCAL1 agent described herein may be incorporated with an excipient and used in the form of ingestible tablets, buccal tablets, troches, capsules, elixirs, suspensions, syrups, and wafers. An anti-SMARCAL1 agent described herein formulated for nasal administration may conveniently be formulated as aerosols, drops, gels, and powders. Aerosol formulations typically include a solution or fine suspension of the active substance in a physiologically acceptable aqueous or non-aqueous solvent and are usually presented in single or multidose quantities in sterile form in a sealed container, which can take the form of a cartridge or refill for use with an atomizing device. Alternatively, the sealed container may be a unitary dispensing device, such as a single-dose nasal inhaler or an aerosol dispenser fitted with a metering valve which is intended for disposal after use. Where the dosage form includes an aerosol dispenser, it will contain a propellant, which can be a compressed gas, such as compressed air or an organic propellant, such as fluorochlorohydrocarbon. The aerosol dosage forms can also take the form of a pump-atomizer. An anti-SMARCAL1 agent described herein formulated for buccal or sublingual administration include tablets, lozenges, and pastilles, where the active ingredient is formulated with a carrier, such as sugar, acacia, tragacanth, gelatin, and glycerine. Anti-SMARCAL1 agents described herein formulated for rectal administration are conveniently in the form of suppositories containing a conventional suppository base, such as cocoa butter.

Dosing

The dosage of the anti-SMARCAL1 agents described herein, and/or compositions including an anti-SMARCAL1 agent described herein, can vary depending on many factors, such as the pharmacodynamic properties of the agent or compound; the mode of administration; the age, health, and weight of the recipient; the nature and extent of the symptoms; the frequency of the treatment, and the type of concurrent treatment, if any; and the clearance rate of the agent or compound in the animal to be treated. One of skill in the art can determine the appropriate dosage based on the above factors. The anti-SMARCAL1 agents described herein may be administered initially in a suitable dosage that may be adjusted as required, depending on the clinical response. In some embodiments, the dosage of a composition (e.g., a composition including an anti-SMARCAL1 agent) is a therapeutically-effective amount.

### Kits

The invention also features kits including (a) a pharmaceutical composition including an agent that reduces the level and/or activity of SMARCAL1 in a cell described herein, and (b) a package insert with instructions to perform any of the methods described herein. In some embodiments, the kit includes (a) a pharmaceutical composition including an agent that reduces the level and/or activity of SMARCAL1 in a cell described herein, (b) an additional therapeutic agent (e.g., an anti-cancer agent), and (c) a package insert with instructions to perform any of the methods described herein.

### EXAMPLES

### Example 1

### Correlation Analysis of sgRNA CRISPR Scores with ATRX Status in Cancer Cell Lines

The following example shows that ATRX deficiency correlates with SMARCAL1 dependency in tumor cells.

Procedure: 390 cancer cell lines where classified according to ATRX deficiency status, (P31FUJ G2292CLONEA141B1, SAOS-22, HS729, and U2OS cell lines were considered to be ATRX-deficient based on literature). PARIS correlation analysis was performed using CERES sgRNA score values.

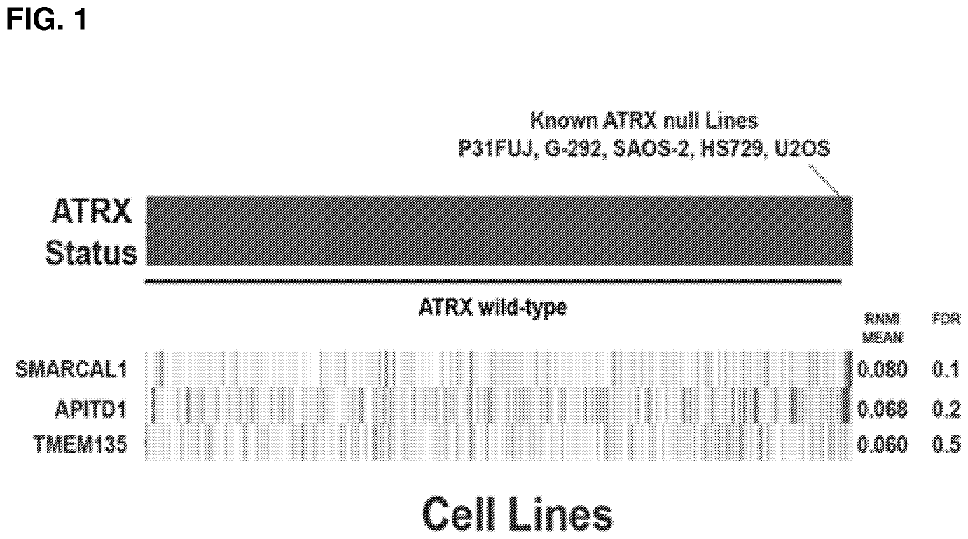

Results: As shown in FIG. 1, the top correlation with ATRX deficiency corresponds to a dependency on SMARCAL1 as accessed by sgRNA CRISPR knock-down.

### Example 2

### High Density Tilling sgRNA Screen Against Human SMARCAL1 in SAOS-2 and G-292 Osteosarcoma Cells

The following example shows that SMARCAL1 sgRNA inhibits cell growth in SAOS-2 and G-292 osteosarcoma cells.

Procedure: To perform high density sgRNA tiling screen, a sgRNA library against SMARCAL1 was custom synthesized at Cellecta (Mountain View, Calif.). Sequences of DNA encoding the SMARCAL1-targeting sgRNAs used in this screen are listed in Table 2. Negative and positive control sgRNA were included in the library. Negative controls consisted of 200 sgRNAs that do not target the human genome. The positive controls include sgRNAs targeting essential genes (CDC16, GTF2B, HSPAS, HSPA9, PAFAH1B1, PCNA, POLR2L, RPL9, and SF3A3). The procedure for virus production, cell infection, and performing sgRNA screen were previously described (Tsherniak et al, Cell 170:564-576 (2017); Munoz et al, Cancer Discovery 6:900-913 (2016)). For each sgRNA, 50 counts were added to the sequencing counts, and for each time point the resulting counts were normalized to the total number of counts. The log2 of the ratio between the counts (defined as dropout ratio) at day 23 for G-292 or day 25 for SAOS-2 and day 1 post-infection was calculated. For negative control sgRNAs, the 2.5 and 97.5 percentile of the log2 dropout ratio of all non-targeting sgRNAs was calculated and considered as background (grey box in the graph). Protein domains were obtained from PFAM regions defined for the UNIPROT identifier Q9NZC9.

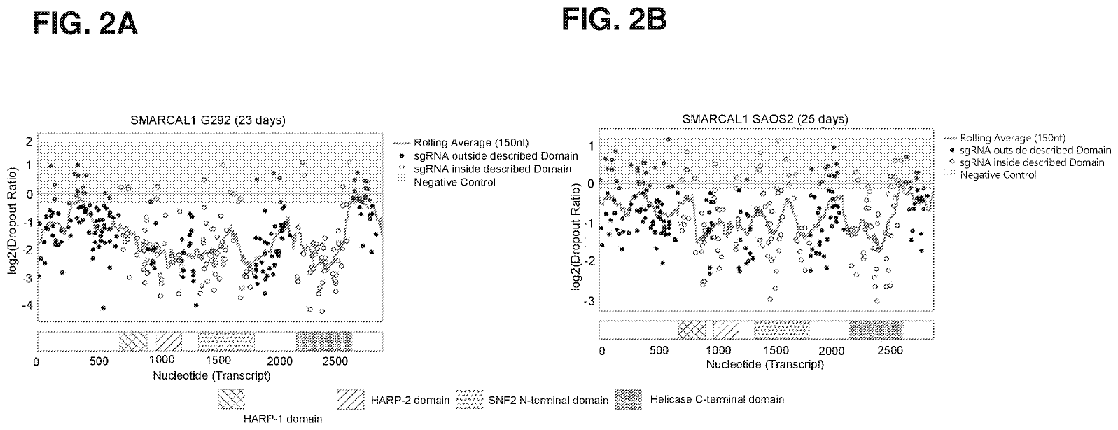

Results: As shown in FIG. 2, targeted inhibition of SMARCAL1 by sgRNA resulted in growth inhibition of the SAOS-2 and G-292 osteosarcoma cell lines. sgRNA against SMARCAL1-described pfam domains showed increased sensitivity in the tested cells. These data suggest that targeting functional domains of SMARCAL1 can be a therapeutic strategy for the treatment of osteosarcoma.

TABLE 2
SMARCAL1 sgRNA Library

| SEQ ID | |
|---|---|
| NO | Nucleic Acid Sequence |
| 46 | CTGCTCCTCTGTAAGAGGCA |
| 47 | TTCCTCTGCTCCTCTGTAAG |
| 48 | TGCCTCTTACAGAGGAGCAG |
| 49 | GATTGAAGAGAATCGACAAA |
| 50 | AGAGAATCGACAAAAGGCTC |
| 51 | CAATAACTTCTCAGCTCTGC |
| 52 | CCAATAACTTCTCAGCTCTG |
| 53 | CCGCAGAGCTGAGAAGTTAT |
| 54 | TATTGGCAGAACAGCATCAG |
| 55 | ACAGCATCAGAGGACTAGCT |
| 56 | CAGCATCAGAGGACTAGCTC |
| 57 | GAATGGGTTGCCAGCAATGG |
| 58 | AGCTCGGGCACCTCCATTGC |
| 59 | CTGGAATGGGTTGCCAGCAA |
| 60 | GGCCTTGCTTGGCCTGGAAT |
| 61 | GGGCCTTGCTTGGCCTGGAA |
| 62 | GGGATGGGCCTTGCTTGGCC |
| 63 | AACCCATTCCAGGCCAAGCA |
| 64 | GCCCATCCCAAAATTTCCCA |
| 65 | CCCATCCCAAAATTTCCCAA |
| 66 | AAAATGACACCATGGCTCAC |
| 67 | TCTTGTAAGCCAGTGAGCCA |
| 68 | GTTGCTTGAAAATGACACCA |
| 69 | TGTGGGAATCATGAGGTCTT |
| 70 | TGAAAACTGTGGGAATCATG |
| 71 | TCCCTTTGCCTGAAAACTGT |
| 72 | TTCCCTTTGCCTGAAAACTG |
| 73 | CAGGCTGTGGGCATTTCTTC |
| 74 | GAAATGCCCACAGCCTGCCC |
| 75 | ACGTGGACTGTGGCCTGGGC |
| 76 | GACTACGTGGACTGTGGCCT |
| 77 | TGACTACGTGGACTGTGGCC |
| 78 | CCATTTGACTACGTGGACTG |
| 79 | GTGAGAGCCATTTGACTACG |
| 80 | CCACAGTCCACGTAGTCAAA |
| 81 | CGTAGTCAAATGGCTCTCAC |
| 82 | CACTGGAATCTCTCCTCCCT |
| 83 | GGAGGACTTTGTGCCAAGGG |
| 84 | TCTGGAGGACTTTGTGCCAA |
| 85 | CTCTGGAGGACTTTGTGCCA |
| 86 | CTTGGCACAAAGTCCTCCAG |
| 87 | TGTTGTTTAGGGACCTCTGG |
| 88 | AGCTGTTGTTTAGGGACCTC |
| 89 | AACTCAAGAGCTGTTGTTTA |
| 90 | TAACTCAAGAGCTGTTGTTT |
| 91 | CAGCTCTTGAGTTATGAGTT |
| 92 | TTGAGTTATGAGTTAGGTCA |
| 93 | GTTAGGTCAAGGTCATGCTC |
| 94 | AAGGGTGTGAACCTGATCTC |
| 95 | CTCAGGCTTCACCTGAGATC |
| 96 | TTATGAGTTGGGTTAGCAAA |
| 97 | CTTATGAGTTGGGTTAGCAA |
| 98 | TGGCCAGAGGCTTATGAGTT |
| 99 | TTGGCCAGAGGCTTATGAGT |
| 100 | TAACCCAACTCATAAGCCTC |
| 101 | GAACTCTTTGGTTTGGCCAG |
| 102 | CTCTTGGGAACTCTTTGGTT |
| 103 | GGTGTCTCTTGGGAACTCTT |
| 104 | GGAATGAGCTGGTGTCTCTT |
| 105 | AGGAATGAGCTGGTGTCTCT |
| 106 | GGCTGTCCAGAGGAATGAGC |
| 107 | GAGACACCAGCTCATTCCTC |
| 108 | ATCCCTGGGAGGCTGTCCAG |
| 109 | ATTCCTCTGGACAGCCTCCC |
| 110 | TTCCTCTGGACAGCCTCCCA |
| 111 | TCTAACTTAGCATCCCTGGG |
| 112 | GCCTCTAACTTAGCATCCCT |
| 113 | GGCCTCTAACTTAGCATCCC |
| 114 | TCCCAGGGATGCTAAGTTAG |
| 115 | AATGTTCTGCCCCGAAGGGG |
| 116 | GACAGCAAAAGCCTCCCCTT |
| 117 | ACAGCAAAAGCCTCCCCTTC |
| 118 | AGAAATGTTCTGCCCCGAAG |
| 119 | AAGAAATGTTCTGCCCCGAA |
| 120 | TAAGAAATGTTCTGCCCCGA |
| 121 | TTACACTCTCTGAGCTAGAA |
| 122 | GCTCAGAGAGTGTAACGCCC |
| 123 | AGTGTAACGCCCAGGACAGA |
| 124 | CTGAGGACCCTGACTTCTGC |
| 125 | TCAGGGTCCTCAGTCCAAAA |
| 126 | AAAAGGAGTGAACTCTCAGA |
| 127 | AAAGGAGTGAACTCTCAGAA |
| 128 | CTCAGAAGGGAAAGTGCGTA |
| 129 | AAGGGAAAGTGCGTAAGGAA |
| 130 | AAGGAACGGCGATCGTTTCC |
| 131 | CATTGTACCCAATCAACACC |
| 132 | GATCGTTTCCAGGTGTTGAT |
| 133 | ATCGTTTCCAGGTGTTGATT |
| 134 | GGTGTTGATTGGGTACAATG |
| 135 | AAGTTCCACGTCTTGGTGTC |
| 136 | CATGCTGAAGTTCCACGTCT |
| 137 | GTTGACCGTGGGGAGGCTCT |
| 138 | GGTTGACCGTGGGGAGGCTC |
| 139 | GCTGCAGGTTGACCGTGGGG |
| 140 | GAGGCTGCAGGTTGACCGTG |
| 141 | AGAGGCTGCAGGTTGACCGT |
| 142 | CAGAGGCTGCAGGTTGACCG |
| 143 | AGGCCCATTCCAGAGGCTGC |
| 144 | CACGGTCAACCTGCAGCCTC |
| 145 | CTGCCATAGGCCCATTCCAG |
| 146 | TCAACCTGCAGCCTCTGGAA |
| 147 | CAACCTGCAGCCTCTGGAAT |
| 148 | CAGCCTCTGGAATGGGCCTA |
| 149 | GGGTGACTCGCTGCTGCCAT |
| 150 | TGTCCCTCACTGCTGGTGGA |
| 151 | CTGTCCCTCACTGCTGGTGG |
| 152 | GGCCTGTCCCTCACTGCTGG |
| 153 | GTCACCCTCCACCAGCAGTG |
| 154 | TCACCCTCCACCAGCAGTGA |
| 155 | CTCCACCAGCAGTGAGGGAC |
| 156 | ACCAGCAGTGAGGGACAGGC |
| 157 | GGATGGAGCTGATGGAAGGC |
| 158 | AAAGGGATGGAGCTGATGGA |
| 159 | AATGAAAGGGATGGAGCTGA |
| 160 | CCTTTGACAAATGAAAGGGA |
| 161 | TCGCCCTTTGACAAATGAAA |
| 162 | ATCGCCCTTTGACAAATGAA |
| 163 | CCATCCCTTTCATTTGTCAA |
| 164 | CATCCCTTTCATTTGTCAAA |
| 165 | GGCGATGCATGCTCATCTCC |
| 166 | GCGATGCATGCTCATCTCCA |
| 167 | GTCTGCCTCGAAGTAGGCCC |
| 168 | ACTGATGTCTGCCTCGAAGT |
| 169 | CATCTCCAGGGCCTACTTCG |
| 170 | GGCAGACATCAGTTATTCAC |
| 171 | TCTGTTTAAAAAGCGCAATA |
| 172 | CAAGAGAAAGCTCCACTTCC |
| 173 | CAGGAAGTGGAGCTTTCTCT |
| 174 | GGTCCAGCTGAACTTGTGGG |
| 175 | GAGGGTCCAGCTGAACTTGT |
| 176 | AGAGGGTCCAGCTGAACTTG |
| 177 | CTGCCTCCCACAAGTTCAGC |
| 178 | GGGTGAGAGTCGTGGGCAGA |
| 179 | AGGGTGAGAGTCGTGGGCAG |
| 180 | AACGCCAGGGTGAGAGTCGT |
| 181 | AAACGCCAGGGTGAGAGTCG |
| 182 | TCTGCCCACGACTCTCACCC |
| 183 | GAGCTGAGAAGCAAACGCCA |
| 184 | TGAGCTGAGAAGCAAACGCC |
| 185 | AGGTCTGCCTCTGGGACATC |
| 186 | TCTCACGCCAGATGTCCCAG |
| 187 | CTTCAGAAAGGTCTGCCTCT |
| 188 | ACTTCAGAAAGGTCTGCCTC |
| 189 | GCTTGGGGTCCACTTCAGAA |
| 190 | AGAGGCAGACCTTTCTGAAG |
| 191 | TCAGATTAGACACGAGCTTG |
| 192 | ATCAGATTAGACACGAGCTT |
| 193 | CATCAGATTAGACACGAGCT |
| 194 | TTGACTCCAGCTCTCTGAAA |
| 195 | CAGGCGGCCTCCTTTGGCTA |
| 196 | GAGCAGCAGGCGGCCTCCTT |
| 197 | TGTCGTCAGCGAGCAGCAGG |
| 198 | CCATGTCGTCAGCGAGCAGC |
| 199 | CCTGCTGCTCGCTGACGACA |
| 200 | CTGCTGCTCGCTGACGACAT |
| 201 | GCTCGCTGACGACATGGGCC |
| 202 | CTCGCTGACGACATGGGCCT |
| 203 | TCGCTGACGACATGGGCCTG |
| 204 | TGCGATGCAGATGGCTTGGA |
| 205 | AGGCTGCGATGCAGATGGCT |
| 206 | GTAAAAGGCTGCGATGCAGA |
| 207 | CGGCCACTCCTTCCGGTAAA |
| 208 | GATGGCACCACCACCAGGAG |
| 209 | GAAGGAGTGGCCGCTCCTGG |
| 210 | CGGAGGATGGCACCACCACC |
| 211 | CAGGTGAAGCGCACGGAGGA |
| 212 | CTCCCAGGTGAAGCGCACGG |
| 213 | CTGCTCCCAGGTGAAGCGCA |
| 214 | CATCCTCCGTGCGCTTCACC |
| 215 | ATCCTCCGTGCGCTTCACCT |
| 216 | CGTGCGCTTCACCTGGGAGC |
| 217 | AGATGGCAGCCACCGAAGGA |
| 218 | TCAGAGATGGCAGCCACCGA |
| 219 | ATGCAATCTGGGCTCAGAGA |
| 220 | CCACGACGTTGATGCAATCT |
| 221 | ACCACGACGTTGATGCAATC |
| 222 | CCCAGATTGCATCAACGTCG |
| 223 | TGCATCAACGTCGTGGTGAC |
| 224 | GCATCAACGTCGTGGTGACT |
| 225 | CAACGTCGTGGTGACTGGGA |
| 226 | TGATCAGGCCAGCTGTCAGG |
| 227 | TGTTGATCAGGCCAGCTGTC |
| 228 | GGGAAGGACCGCCTGACAGC |
| 229 | CAAAGCTGACAATGTTGATC |
| 230 | CTTTGACCTTCTTAGCAAGT |
| 231 | AATGATGACAACTTTAAAAG |
| 232 | CAATGATGACAACTTTAAAA |
| 233 | CTCACTTCCTCAAAAACAGT |
| 234 | CGGCATAGCTGCTCGACAGC |
| 235 | CCGGCATAGCTGCTCGACAG |
| 236 | CCGCTGTCGAGCAGCTATGC |
| 237 | AGCAGCTATGCCGGTCCTAA |
| 238 | CGACAACAGGATCACCCTCT |
| 239 | TGGCTGGTGTGCCCGACAAC |
| 240 | AAGAGGGTGATCCTGTTGTC |
| 241 | ATCTGCGTGTAGAGCTCTGC |
| 242 | GATCTGCGTGTAGAGCTCTG |
| 243 | TGAAACTGGGGGAAGAAAGT |
| 244 | GTCCAAAGGCATGAAACTGG |
| 245 | AGTCCAAAGGCATGAAACTG |
| 246 | AAGTCCAAAGGCATGAAACT |
| 247 | GAAGTCCAAAGGCATGAAAC |
| 248 | TTCCCCCAGTTTCATGCCTT |
| 249 | ATCACAGTAGCGAAGTCCAA |
| 250 | TTCGCTACTGTGATGCCAAA |
| 251 | CCTGAGTAGTCCCACCCCCA |
| 252 | CCTTGGGGGTGGGACTACTC |
| 253 | CTTCAGCTCTCCCAGGTTGG |
| 254 | CTACTCAGGTTCCTCCAACC |
| 255 | TACTCAGGTTCCTCCAACCT |
| 256 | GAGCTTCAGCTCTCCCAGGT |
| 257 | GCAGGAGCTTCAGCTCTCCC |
| 258 | GGGAGAGCTGAAGCTCCTGC |
| 259 | GCATGACTGCTTCCTCCAGC |
| 260 | TGGAGGAAGCAGTCATGCTG |
| 261 | GGGAAAGGACGTCGGACTTG |
| 262 | AGGCAGCTGGGAAAGGACGT |
| 263 | GCTTGGCAGGCAGCTGGGAA |
| 264 | TGCGCTGCTTGGCAGGCAGC |
| 265 | ACTATCTTGCGCTGCTTGGC |
| 266 | CACCACTATCTTGCGCTGCT |
| 267 | TGCCAAGCAGCGCAAGATAG |
| 268 | AAGATAGTGGTGATTGCCCC |
| 269 | CCTGGCATTGATCCGTCCTG |
| 270 | TCCTGGCATTGATCCGTCCT |
| 271 | GTCCTGGCATTGATCCGTCC |
| 272 | CCCCAGGACGGATCAATGCC |
| 273 | ATCCAGGGCAGCTCTGGTCC |
| 274 | TGCAGCATCCAGGGCAGCTC |
| 275 | TGCCAGGACCAGAGCTGCCC |
| 276 | TTCCTTGGCTGCAGCATCCA |
| 277 | TTTCCTTGGCTGCAGCATCC |
| 278 | TGCCCTGGATGCTGCAGCCA |
| 279 | GTCCTTGGTGGTCATTTCCT |
| 280 | AGCCAAGGAAATGACCACCA |
| 281 | TCTGTTGAAGAAGAGAATGA |
| 282 | TGAATATATCTTGGACCTAC |
| 283 | TCTCTCTTCCACTTTCCAGT |
| 284 | ATCTTGGACCTACTGGAAAG |
| 285 | TTTAGTATTTGCACACCATA |
| 286 | TTGCGTCCAGGACCACCTTA |
| 287 | TGCACACCATAAGGTGGTCC |
| 288 | GCTCTTGCGTAATTGCGTCC |
| 289 | ATGAGGTGGAGCCATCGATG |
| 290 | GTGCAGCACATCCGCATCGA |
| 291 | CTCCCGCTCAGCTGATGAGG |
| 292 | GTCCTCCCGCTCAGCTGATG |
| 293 | GCTCCACCTCATCAGCTGAG |
| 294 | CTCCACCTCATCAGCTGAGC |
| 295 | CACCTCATCAGCTGAGCGGG |
| 296 | ACAGTTGGAACTGCTGGCAC |
| 297 | TCTCCGACAGTTGGAACTGC |
| 298 | CAGCATGCCTCTCCGACAGT |
| 299 | AGCAGTTCCAACTGTCGGAG |
| 300 | ACTGTCGGAGAGGCATGCTG |
| 301 | GGCAGCGGTGATGGACAGCA |
| 302 | GCCCATATTGGCAGCGGTGA |
| 303 | GGTGAGGCCCATATTGGCAG |
| 304 | GTCCATCACCGCTGCCAATA |
| 305 | TCCATCACCGCTGCCAATAT |
| 306 | GGAGAAGGTGAGGCCCATAT |
| 307 | GGTCAGCCGAGGAGAAGGTG |
| 308 | CACCAGGTCAGCCGAGGAGA |
| 309 | TATGGGCCTCACCTTCTCCT |
| 310 | AAACACCACCAGGTCAGCCG |
| 311 | CACCTTCTCCTCGGCTGACC |
| 312 | CTTCTCCTCGGCTGACCTGG |
| 313 | ACAGCTCAGCAAACACCACC |
| 314 | TCTGTCCAATGCGGTGCACG |
| 315 | GAGGACCGCGTGCACCGCAT |
| 316 | CGGAGCTGGTCTGTCCAATG |
| 317 | GTAGTGAATGCCCACGGAGC |
| 318 | CATTGGACAGACCAGCTCCG |
| 319 | ATTGGACAGACCAGCTCCGT |
| 320 | CACGAGGTAGTGAATGCCCA |
| 321 | CGTGGGCATTCACTACCTCG |
| 322 | CAGCTGTGCCCTTTGCCACG |
| 323 | CATTCACTACCTCGTGGCAA |
| 324 | ATTCACTACCTCGTGGCAAA |
| 325 | GCACAGCTGATGACTACCTT |
| 326 | TTAATCTTCTCTTGAATCAG |
| 327 | TTTAATCTTCTCTTGAATCA |
| 328 | CTTTAATCTTCTCTTGAATC |
| 329 | TCAAGAGAAGATTAAAGTTC |
| 330 | ATTAAAGTTCTGGCAGAAGC |
| 331 | TTAAAGTTCTGGCAGAAGCC |
| 332 | TTCTGTCATTTCTGAAAAAT |
| 333 | ATCCACTGATTACCTCTACA |
| 334 | CGTAGATCTTCTGCTGCTTT |
| 335 | TCGTAGATCTTCTGCTGCTT |
| 336 | TCTCAAAGGACTTCTGGAAT |
| 337 | CTTCTTTCTCAAAGGACTTC |
| 338 | ATCACTTCCTTCTTTCTCAA |
| 339 | CAGAAGTCCTTTGAGAAAGA |
| 340 | TGAGAAAGAAGGAAGTGATA |
| 341 | AGGAAGTGATATGGAGCTCC |
| 342 | CAAAGGACTCTGCTGCTTCC |
| 343 | TGAAGCACTTCCTGGGTCAA |
| 344 | GCAGCAGAGTCCTTTGACCC |
| 345 | ATGTTCCTGAAGCACTTCCT |
| 346 | GATGTTCCTGAAGCACTTCC |
| 347 | TTTGACCCAGGAAGTGCTTC |
| 348 | GGAAGTGCTTCAGGAACATC |
| 349 | CAGGGTGTCTCCCATGTTCT |
| 350 | CCAGGGTGTCTCCCATGTTC |
| 351 | GGAAGTAGTTCCCAGAACAT |
| 352 | CCAGAACATGGGAGACACCC |
| 353 | TGTCAATGAGCTTTCATCCA |
| 354 | CTGTCAATGAGCTTTCATCC |
| 355 | CAGCCAGTCCACAGAAGAAA |

### Example 3

### Validation of SMARCAL1 Genetic Dependency in ATRX Null/ALT+Osteosarcoma Cell Line SAOS2

The following example shows that SMARCAL1 siRNA inhibits cell growth in SAOS-2 osteosarcoma cells.

Procedure: SAOS2 cell line was seeded in 100 μL of media in 96-well plates (Corning 3904) excluding edge wells at the following density of 10000 cells per well. SOAS2 cells were seeded the day prior to transduction. Custom Stealth siRNA and Negative Control siRNA were ordered from ThermoFisher. The procedure for siRNA delivery was conducted based on the manufacturer's recommendation. The SMARCAL1 targeting siRNA sequence is: UUUCACAGAGAAAUGCUUAUGCAGG (SEQ ID NO: 356).

Cell viability was assayed using CellTiter-Glo (Promega G7572) at 100 μL per well. Luminescence was read using PerkinElmer EnVision 2105. Values were normalized to the average values from the negative control sgRNAs. Experiments were performed three times. Shown are the triplicate results of one representative experiment. Two tailed, type 3 Student's t-test was performed to determine statistical significance, which was conducted on Microsoft Excel (Microsoft).

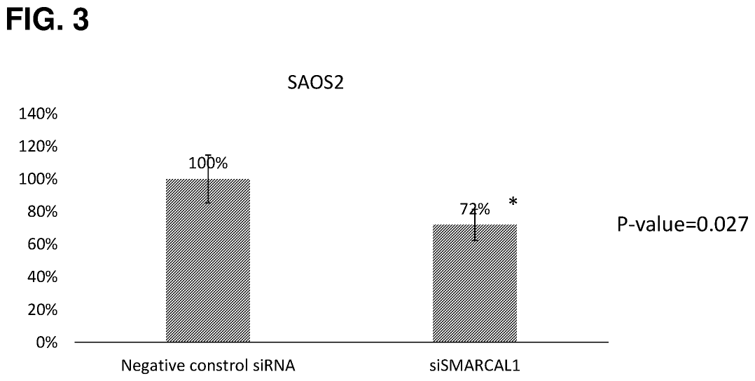

Results: As shown in FIG. 3, treatment of ATRX null/ALT+osteosarcoma cell line SAOS2 with siRNA targeting SMARCAL-1 resulted in decreased viability compared to treatment with a negative control siRNA.

### Example 4

### Validation of SMARCAL1 Genetic Dependency in ATRX Null/ALT+Osteosarcoma Cell Line CAL72

The following example shows that SMARCAL1 shRNA inhibits cell growth in CAL72 osteosarcoma cells.

Procedure: Cells were transduced with lentivirus expressing indicated shRNAs. The shRNA sequences were:

shNT:
(SEQ ID NO: 357)
CAACAAGATGAAGAGCACCAA;
shSMARCAL1-1:
(SEQ ID NO: 358)
CCATCAGCTCCATCCCTTTCA;
and
shSMARCAL1-3:
(SEQ ID NO: 359)
AGGTGTTGATTGGGTACAATG.

24 hours later, medium was replaced with 2 μg/mL puromycin containing medium. After 2-4 days or puromycin selection, negative control cells (cells didn't treat with lentivirus) all died, this indicated the completion of puromycin selection. After the completion of puromycin selection, lentivirus-infected cells were then trypsinized and reseeded onto a 6 well plate. The number of cells seeded per well was 2500, 5000, and 10,000. Cells were subsequently propagated for 2 weeks in puromycin-free media, with or without 0.5 uM doxycycline. Culture medium was changed every 3 days. Crystal violet staining was done with a mixture of 0.5% crystal violet in 50/50 methanol/water for 30 min. Unbound crystal violet was rinsed with de-ionized water and plates were left for drying at room temperature.

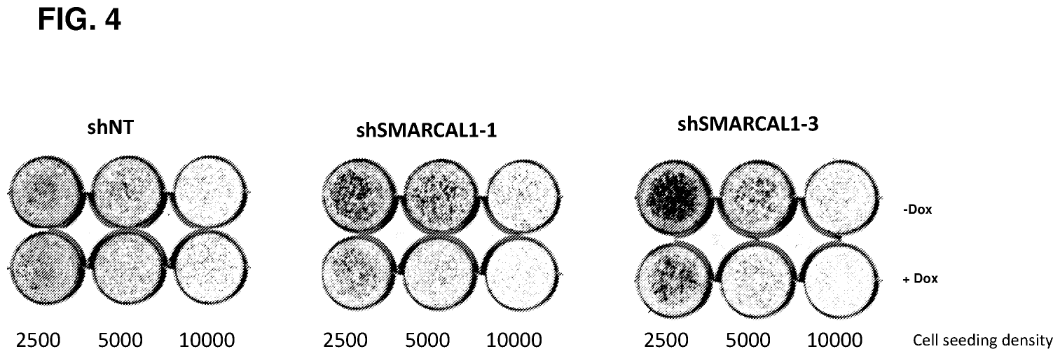

Results: As shown in FIG. 4, treatment of ATRX null/ALT+osteosarcoma cell line CAL72 with two different shRNA targeting SMARCAL-1 resulted in decreased viability compared to treatment with a negative control shRNA.

### Example 5

### Validation of SMARCAL1 Genetic Dependency in ATRX Null/ALT+Astrocytoma Cell Line TM31

The following example shows that SMARCAL1 shRNA inhibits cell growth in TM31 astrocytoma cells.

Procedure: Cells were transduced with lentivirus expressing indicated shRNAs (see Example 4). 24 hours later, medium was replaced with 2 μg/mL puromycin containing medium. After 2-4 days or puromycin selection, negative control cells (cells didn't treat with lentivirus) all died, this indicated the completion of puromycin selection. After the completion of puromycin selection, lentivirus-infected cells were then trypsinized and reseeded onto a 6 well plate. The number of cells seeded per well was 1000, 2000, and 4000. Cells were subsequently propagated for 2 weeks in puromycin-free media, with or without 0.5 uM doxycycline. Culture medium was changed every 3 days. Crystal violet staining was done with a mixture of 0.5% crystal violet in 50/50 methanol/water for 30 min. Unbound crystal violet was rinsed with de-ionized water and plates were left for drying at room temperature.

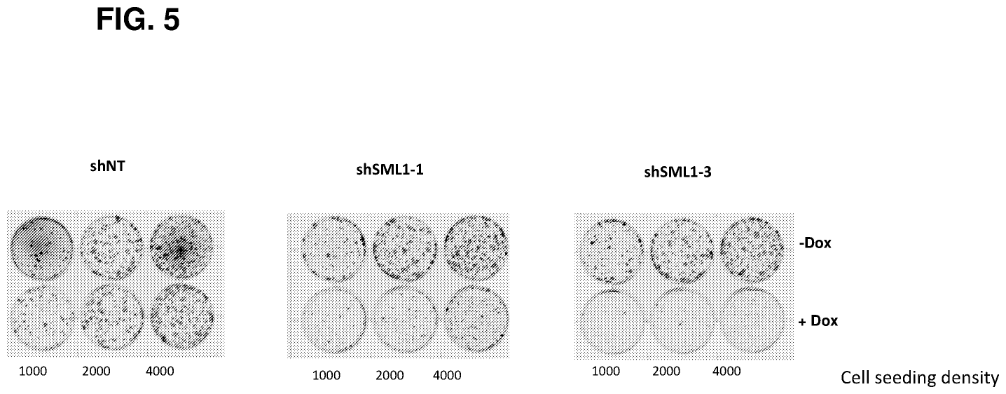

Results: As shown in FIG. 5, treatment of ATRX null/ALT+astrocytoma cell line TM31 with two different shRNA targeting SMARCAL-1 resulted in decreased viability compared to treatment with a negative control shRNA.

### Example 6

### Validation of SMARCAL1 Genetic Dependency in Alveolar Rhabdomyosarcoma Cell Line RH30 and ATRX Null Fibroblast Cell Line HS729

The following example shows that SMARCAL1 shRNA inhibits cell growth in RH30 rhabdomyosarcoma cells and HS729 fibroblast cells.

Procedure: Cells were transduced with lentivirus expressing indicated shRNAs (see Example 4). 24 hours later, medium was replaced with 2 μg/mL puromycin containing medium. After 2-4 days or puromycin selection, negative control cells (cells didn't treat with lentivirus) all died, this indicated the completion of puromycin selection. After the completion of puromycin selection, lentivirus-infected cells were then trypsinized and reseeded onto a 6 well plate. The number of cells seeded per well was 1000, 2000, and 4000. Cells were subsequently propagated for 2 weeks in puromycin-free media, with or without 0.5 uM doxycycline. Culture medium was changed every 3 days. Crystal violet staining was done with a mixture of 0.5% crystal violet in 50/50 methanol/water for 30 min. Unbound crystal violet was rinsed with de-ionized water and plates were left for drying at room temperature.

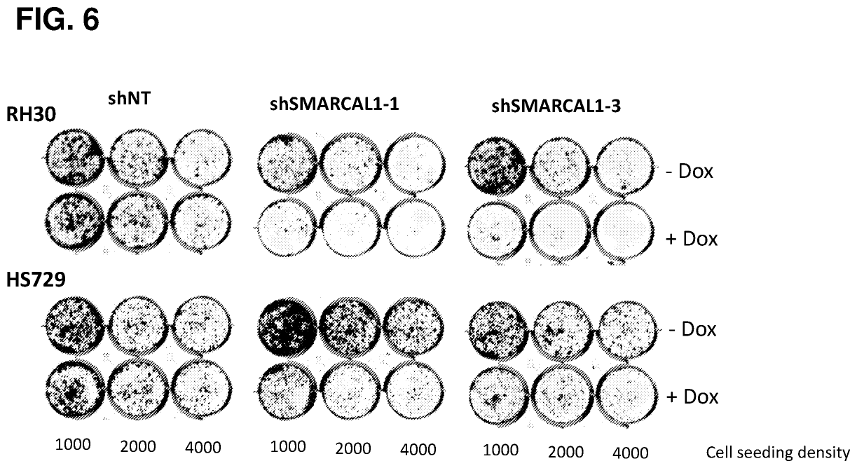

Results: As shown in FIG. 6, treatment of alveolar rhabdomyosarcoma cell line RH30 and ATRX null fibroblast cell line HS729 with two different shRNA targeting SMARCAL-1 resulted in decreased viability compared to treatment with a negative control shRNA.

### Other Embodiments

All publications, patents, and patent applications mentioned in this specification are incorporated herein by reference in their entirety to the same extent as if each individual publication, patent, or patent application was specifically and individually indicated to be incorporated by reference in its entirety. Where a term in the present application is found to be defined differently in a document incorporated herein by reference, the definition provided herein is to serve as the definition for the term.

While the invention has been described in connection with specific embodiments thereof, it will be understood that invention is capable of further modifications and this application is intended to cover any variations, uses, or adaptations of the invention following, in general, the principles of the invention and including such departures from the present disclosure that come within known or customary practice within the art to which the invention pertains and may be applied to the essential features hereinbefore set forth, and follows in the scope of the claimed.

1. A method of treating an alternative lengthening of telomeres (ALT)-positive cancer or a cancer having a mutation that results in a loss of function of ATRX and/or DAXX in a subject in need thereof, the method comprising administering to the subject an effective amount of an agent that reduces the level and/or activity of SWI/SNF-related matrix-associated actin-dependent regulator of chromatin subfamily A-like protein 1 (SMARCAL1) in the subject.

2. A method of reducing the level and/or activity of SMARCAL1 in an ALT-positive cancer cell or a cancer cell having a mutation that results in a loss of function of ATRX and/or DAXX in a subject, the method comprising contacting the cell with an effective amount of an agent that reduces the level and/or activity of SMARCAL1 in the cell.

3. A method of reducing tumor growth of an ALT-positive-cancer or a cancer having a mutation that results in a loss of function of ATRX and/or DAXX in a subject, the method comprising administering to the subject an effective amount of an agent that reduces the level and/or activity of SMARCAL1 in a cell in the subject.

4. The method of claim 1, wherein the ALT-positive-cancer is associated with a mutation in the ATRX gene or DAXX gene.

5. (canceled)

6. The method of claim 4, wherein the mutation in the ATRX gene is a mutation that results in a loss of function of ATRX or DAXX gene.

7-10. (canceled)

11. The method of claim 1, wherein the agent that reduces the level and/or activity of SMARCAL1 is a nuclease, a polynucleotide, a small-molecule compound, an antibody, and/or an enzyme.

12. The method of claim 11, wherein the agent that reduces the level and/or activity of SMARCAL1 is a nuclease that is a clustered regularly interspaced short palindromic repeats (CRISPR)-associated protein, a transcription activator-like effector nuclease (TALEN), a meganuclease, or a zinc finger nuclease (ZFN).

13. (canceled)

14. The method of claim 12, wherein the CRISPR-associated protein is CRISPR-associated protein 9 (Cas9).

15. (canceled)

16. The method of claim 11, wherein the agent that reduces the level and/or activity of SMARCAL1 is a polynucleotide.

17. The method of claim 16, wherein the polynucleotide is an antisense nucleic acid, a CRISPR/Cas 9 nucleotide, a short interfering RNA (siRNA), a short hairpin RNA (shRNA), a micro RNA (miRNA), or a ribozyme.

18-19. (canceled)

20. The method of claim 1, wherein the method further comprises administering to the subject an anti-cancer therapy.

21. The method of claim 20, wherein the anti-cancer therapy is a telomerase inhibitor or a small molecule that induces DNA damage and/or modulates a DNA-repair pathway and/or a replication stress pathway;
wherein the anti-cancer therapy is doxorubicin, cisplatin, ifosfamide, or high-dose methotrexate (MTX) with leucovorin rescue, or a combination thereof;
wherein the anti-cancer therapy is surgery, temozolomide, radiation therapy, procarbazine, lomustine, or vincristine, or a combination thereof;
wherein the anti-cancer therapy is surgery, radiation therapy, temozolomide, carmustine, lomustine, cisplatin, procarbazine, or vincristine, or a combination thereof; or
wherein the anti-cancer therapy is vincristine, dactinomycin, or cyclophosphamide, or a combination thereof.

22-25. (canceled)

26. The method of claim 1, wherein the agent that reduces the level and/or of SMARCAL1 is administered systemically or intratumorally to the subject.

27. The method of claim 1, wherein the subject has a cancer that is refractory to an anti-cancer therapy.

28. The method of claim 1, wherein the cancer is a soft tissue sarcoma, an osteosarcoma, a pancreatic neuroendocrine tumor (PanNET), a glioma, a glioblastoma, a pediatric glioblastoma, an astrocytoma, an endometrial cancer, an adrenocortical carcinoma, a neuroepithelial tumor, a non-small cell lung cancer, a bladder cancer, an esophagogastric cancer, a melanoma, a head and neck cancer, a cervical cancer, a Non-Hodgkin lymphoma, a colorectal cancer, a pancreatic cancer, a germ cell tumor, a breast cancer, an ovarian cancer, a hepatobiliary cancer, a renal cell carcinoma, a pheochromocytoma, a prostate cancer, a thyroid cancer, an adrenal gland/peripheral nervous system cancer, a central nervous system cancer, a gall bladder cancer, a hematopoietic neoplasm, a larynx cancer, a liver cancer, an oral cavity cancer, a pleural cancer, a salivary gland carcinoma, a skin cancer, a small intestine cancer, a stomach cancer, a tendon sheath cancer, a testicular cancer, or a uterine cancer.

29. The method of claim 28, wherein the cancer is an osteosarcoma, an astrocytoma, a PanNET, a soft tissue sarcoma, or a glioblastoma.

30. The method of claim 1, wherein the cancer is a ganglioneuroblastoma, a diffuse astrocytoma, an anaplastic astrocytoma, a glioblastoma multiforme, an oligodendroglioma, an anaplastic medulloblastoma, a paraganglioma, an undifferentiated pleomorphic sarcoma, a fibrosarcoma, a leiomyosarcoma, a liposarcoma, an angiosargoma, an epithelioid sarcoma, a rhabdomyosarcoma, or a nonseminoumatous germ cell tumor.

31. The method of claim 1, wherein the subject is a human.

32. A method of reducing growth of an ALT-positive cancer cell or a cancer cell having a mutation that results in a loss of function of ATRX and/or DAXX, the method comprising contacting the cell with an effective amount of an agent that reduces the level and/or activity of SMARCAL1 in the cell.

33. (canceled)

34. The method of claim 32, wherein the cancer cell is a soft tissue sarcoma cell, an osteosarcoma cell, a PanNET cell, a glioma cell, a glioblastoma cell, a pediatric glioblastoma cell, an astrocytoma cell, an endometrial cancer cell, an adrenocortical carcinoma cell, a neuroepithelial tumor cell, a non-small cell lung cancer cell, a bladder cancer cell, an esophagogastric cancer cell, a melanoma cell, a head and neck cancer cell, a cervical cancer cell, a Non-Hodgkin lymphoma cell, a colorectal cancer cell, a pancreatic cancer cell, a germ cell tumor cell, a breast cancer cell, an ovarian cancer cell, a hepatobiliary cancer cell, a renal cell carcinoma cell, a pheochromocytoma cell, a prostate cancer cell, a thyroid cancer cell, an adrenal gland/peripheral nervous system cancer cell, a central nervous system cancer cell, a gall bladder cancer cell, a hematopoietic neoplasm cell, a larynx cancer cell, a liver cancer cell, an oral cavity cancer cell, a pleural cancer cell, a salivary gland carcinoma cell, a skin cancer cell, a small intestine cancer cell, a stomach cancer cell, a tendon sheath cancer cell, a testicular cancer cell, a uterine cancer cell, a ganglioneuroblastoma cell, a diffuse astrocytoma cell, an anaplastic astrocytoma cell, a glioblastoma multiforme cell, an oligodendroglioma cell, an anaplastic medulloblastoma cell, a paraganglioma cell, an undifferentiated pleomorphic sarcoma cell, a fibrosarcoma cell, a leiomyosarcoma cell, a liposarcoma cell, an angiosargoma cell, an epithelioid sarcoma cell, or a nonseminoumatous germ cell tumor cell.

35. The method of claim 34, wherein the cancer cell is an osteosarcoma cell, an astrocytoma, a PanNET cell, a soft tissue sarcoma cell, or a glioblastoma cell.


SEQUENCE LISTING (359 sequences)

SEQ ID NO: 1 - protein, 954 aa, Homo sapiens
MSLPLTEEQRKKIEENRQKALARRAEKLLAEQHQRTSSGTSIAGNPFQAKQGPSQNFPRESCKPVSHGVIFKQQNLSSSSNADQRPHDSHSFQAKGIWKKPEEMPTACPGHSPRSQMALTGISPPLAQSPPEVPKQQLLSYELGQGHAQASPEIRFTPFANPTHKPLAKPKSSQETPAHSSGQPPRDAKLEAKTAKASPSGQNISYIHSSSESVTPRTEGRLQQKSGSSVQKGVNSQKGKCVRNGDRFQVLIGYNAELIAVFKTLPSKNYDPDTKTWNFSMNDYSALMKAAQSLPTVNLQPLEWAYGSSESPSTSSEGQAGLPSAPSLSFVKGRCMLISRAYFEADISYSQDLIALFKQMDSRRYDVKTRKWSFLLEEHSKLIAKVRCLPQVQLDPLPTTLTLAFASQLKKTSLSLTPDVPEADLSEVDPKLVSNLMPFQRAGVNFAIAKGGRLLLADDMGLGKTIQAICIAAFYRKEWPLLVVVPSSVRFTWEQAFLRWLPSLSPDCINVVVTGKDRLTAGLINIVSFDLLSKLEKQLKTPFKVVIIDESHFLKNSRTARCRAAMPVLKVAKRVILLSGTPAMSRPAELYTQIIAVKPTFFPQFHAFGLRYCDAKRMPWGWDYSGSSNLGELKLLLEEAVMLRRLKSDVLSQLPAKQRKIVVIAPGRINARTRAALDAAAKEMTTKDKTKQQQKDALILFFNRTAEAKIPSVIEYILDLLESGREKFLVFAHHKVVLDAITQELERKHVQHIRIDGSTSSAEREDLCQQFQLSERHAVAVLSITAANMGLTFSSADLVVFAELFWNPGVLIQAEDRVHRIGQTSSVGIHYLVAKGTADDYLWPLIQEKIKVLAEAGLSETNFSEMTESTDYLYKDPKQQKIYDLFQKSFEKEGSDMELLEAAESFDPGSASGTSGSSSQNMGDTLDESSLTASPQKKRRFEFFDNWDSFTSPL

SEQ ID NO: 2 - DNA, 3095 bp, Homo sapiens
gatcgtaggggtgggagtggggcgtggcgcccgcttaccttgaggctgggttggaaaaagactatgttagcaagtgtcacgccatgcttttgccaactttccaattaaaggttgacattcctgcataagcatttctctgtgaaaatgtccttgcctcttacagaggagcagaggaaaaagattgaagagaatcgacaaaaggctctggcccgcagagctgagaagttattggcagaacagcatcagaggactagctcgggcacctccattgctggcaacccattccaggccaagcaaggcccatcccaaaatttcccaagggagtcttgtaagccagtgagccatggtgtcattttcaagcaacagaatctcagtagctcatctaatgctgaccaaagacctcatgattcccacagttttcaggcaaagggaatatggaaaaagccagaagaaatgcccacagcctgcccaggccacagtccacgtagtcaaatggctctcactggaatctctcctcccttggcacaaagtcctccagaggtccctaaacaacagctcttgagttatgagttaggtcaaggtcatgctcaggcttcacctgagatcaggttcacaccctttgctaacccaactcataagcctctggccaaaccaaagagttcccaagagacaccagctcattcctctggacagcctcccagggatgctaagttagaggccaagacagcaaaagcctccccttcggggcagaacatttcttacatccattctagctcagagagtgtaacgcccaggacagaaggaagactccagcagaagtcagggtcctcagtccaaaaaggagtgaactctcagaagggaaagtgcgtaaggaacggcgatcgtttccaggtgttgattgggtacaatgcggaactcattgcagtgtttaagaccctgcccagcaagaattatgatcctgacaccaagacgtggaacttcagcatgaatgactatagtgccctgatgaaagcagcccagagcctccccacggtcaacctgcagcctctggaatgggcctatggcagcagcgagtcaccctccaccagcagtgagggacaggccggccttccatcagctccatccctttcatttgtcaaagggcgatgcatgctcatctccagggcctacttcgaggcagacatcagttattcacaggaccttattgcgctttttaaacagatggattccagaagatatgatgtcaagaccaggaagtggagctttctcttggaagagcacagtaaactaattgcaaaggtgcgctgcctcccacaagttcagctggaccctctgcccacgactctcaccctggcgtttgcttctcagctcaagaagacatctctcagtctcacgccagatgtcccagaggcagacctttctgaagtggaccccaagctcgtgtctaatctgatgccctttcagagagctggagtcaattttgccatagccaaaggaggccgcctgctgctcgctgacgacatgggcctggggaagaccatccaagccatctgcatcgcagccttttaccggaaggagtggccgctcctggtggtggtgccatcctccgtgcgcttcacctgggagcaggccttccttcggtggctgccatctctgagcccagattgcatcaacgtcgtggtgactgggaaggaccgcctgacagctggcctgatcaacattgtcagctttgaccttcttagcaagttggaaaaacagctaaaaaccccttttaaagttgtcatcattgatgaatctcacttcctcaaaaacagtaggactgcccgctgtcgagcagctatgccggtcctaaaggttgccaagagggtgatcctgttgtcgggcacaccagccatgtcccggcccgcagagctctacacgcagatcatcgcagtcaagccaactttcttcccccagtttcatgcctttggacttcgctactgtgatgccaaacggatgccttgggggtgggactactcaggttcctccaacctgggagagctgaagctcctgctggaggaagcagtcatgctgcggcgcctcaagtccgacgtcctttcccagctgcctgccaagcagcgcaagatagtggtgattgccccaggacggatcaatgccaggaccagagctgccctggatgctgcagccaaggaaatgaccaccaaggacaaaactaaacagcagcagaaagatgccctcattctcttcttcaacagaacagctgaagctaaaatcccatctgtcattgaatatatcttggacctactggaaagtggaagagagaagtttttagtatttgcacaccataaggtggtcctggacgcaattacgcaagagcttgagagaaagcacgtgcagcacatccgcatcgatggctccacctcatcagctgagcgggaggacctgtgccagcagttccaactgtcggagaggcatgctgtggccgtgctgtccatcaccgctgccaatatgggcctcaccttctcctcggctgacctggtggtgtttgctgagctgttttggaacccaggggtgctgatccaggctgaggaccgcgtgcaccgcattggacagaccagctccgtgggcattcactacctcgtggcaaagggcacagctgatgactacctttggcccctgattcaagagaagattaaagttctggcagaagccgggctttctgagaccaatttttcagaaatgacagaatccactgattacctctacaaggacccaaagcagcagaagatctacgacctattccagaagtcctttgagaaagaaggaagtgatatggagctcctggaagcagcagagtcctttgacccaggaagtgcttcaggaacatctggaagtagttcccagaacatgggagacaccctggatgaaagctcattgacagccagtccacagaagaaaaggagatttgaattttttgataactgggacagctttacgtctcccctgtaaaaggggcaaaaagaaaaaaataaaaagcattttaaaatcatggaattgaaataaaataatgtattttgttttaaaaaaaaaaaaaa

SEQ ID NO: 3 - protein, 2492 aa, Homo sapiens
MTAEPMSESKLNTLVQKLHDFLAHSSEESEETSSPPRLAMNQNTDKISGSGSNSDMMENSKEEGTSSSEKSKSSGSSRSKRKPSIVTKYVESDDEKPLDDETVNEDASNENSENDITMQSLPKGTVIVQPEPVLNEDKDDFKGPEFRSRSKMKTENLKKRGEDGLHGIVSCTACGQQVNHFQKDSIYRHPSLQVLICKNCFKYYMSDDISRDSDGMDEQCRWCAEGGNLICCDFCHNAFCKKCILRNLGRKELSTIMDENNQWYCYICHPEPLLDLVTACNSVFENLEQLLQQNKKKIKVDSEKSNKVYEHTSRFSPKKTSSNCNGEEKKLDDSCSGSVTYSYSALIVPKEMIKKAKKLIETTANMNSSYVKFLKQATDNSEISSATKLRQLKAFKSVLADIKKAHLALEEDLNSEFRAMDAVNKEKNTKEHKVIDAKFETKARKGEKPCALEKKDISKSEAKLSRKQVDSEHMHQNVPTEEQRTNKSTGGEHKKSDRKEEPQYEPANTSEDLDMDIVSVPSSVPEDIFENLETAMEVQSSVDHQGDGSSGTEQEVESSSVKLNISSKDNRGGIKSKTTAKVTKELYVKLTPVSLSNSPIKGADCQEVPQDKDGYKSCGLNPKLEKCGLGQENSDNEHLVENEVSLLLEESDLRRSPRVKTTPLRRPTETNPVTSNSDEECNETVKEKQKLSVPVRKKDKRNSSDSAIDNPKPNKLPKSKQSETVDQNSDSDEMLAILKEVSRMSHSSSSDTDINEIHTNHKTLYDLKTQAGKDDKGKRKRKSSTSGSDFDTKKGKSAKSSIISKKKRQTQSESSNYDSELEKEIKSMSKIGAARTTKKRIPNTKDFDSSEDEKHSKKGMDNQGHKNLKTSQEGSSDDAERKQERETFSSAEGTVDKDTTIMELRDRLPKKQQASASTDGVDKLSGKEQSFTSLEVRKVAETKEKSKHLKTKTCKKVQDGLSDIAEKFLKKDQSDETSEDDKKQSKKGTEEKKKPSDFKKKVIKMEQQYESSSDGTEKLPEREEICHFPKGIKQIKNGTTDGEKKSKKIRDKTSKKKDELSDYAEKSTGKGDSCDSSEDKKSKNGAYGREKKRCKLLGKSSRKRQDCSSSDTEKYSMKEDGCNSSDKRLKRIELRERRNLSSKRNTKEIQSGSSSSDAEESSEDNKKKKQRTSSKKKAVIVKEKKRNSLRTSTKRKQADITSSSSSDIEDDDQNSIGEGSSDEQKIKPVTENLVLSSHTGFCQSSGDEALSKSVPVTVDDDDDDNDPENRIAKKMLLEEIKANLSSDEDGSSDDEPEEGKKRTGKQNEENPGDEEAKNQVNSESDSDSEESKKPRYRHRLLRHKLTVSDGESGEEKKTKPKEHKEVKGRNRRKVSSEDSEDSDFQESGVSEEVSESEDEQRPRTRSAKKAELEENQRSYKQKKKRRRIKVQEDSSSENKSNSEEEEEEKEEEEEEEEEEEEEEEDENDDSKSPGKGRKKIRKILKDDKLRTETQNALKEEEERRKRIAEREREREKLREVIEIEDASPTKCPITTKLVLDEDEETKEPLVQVHRNMVIKLKPHQVDGVQFMWDCCCESVKKTKKSPGSGCILAHCMGLGKTLQVVSFLHTVLLCDKLDFSTALVVCPLNTALNWMNEFEKWQEGLKDDEKLEVSELATVKRPQERSYMLQRWQEDGGVMIIGYEMYRNLAQGRNVKSRKLKEIFNKALVDPGPDFVVCDEGHILKNEASAVSKAMNSIRSRRRIILTGTPLQNNLIEYHCMVNFIKENLLGSIKEFRNRFINPIQNGQCADSTMVDVRVMKKRAHILYEMLAGCVQRKDYTALTKFLPPKHEYVLAVRMTSIQCKLYQYYLDHLTGVGNNSEGGRGKAGAKLFQDFQMLSRIWTHPWCLQLDYISKENKGYFDEDSMDEFIASDSDETSMSLSSDDYTKKKKKGKKGKKDSSSSGSGSDNDVEVIKVWNSRSRGGGEGNVDETGNNPSVSLKLEESKATSSSNPSSPAPDWYKDFVTDADAEVLEHSGKMVLLFEILRMAEEIGDKVLVFSQSLISLDLIEDFLELASREKTEDKDKPLIYKGEGKWLRNIDYYRLDGSTTAQSRKKWAEEFNDETNVRGRLFIISTKAGSLGINLVAANRVIIFDASWNPSYDIQSIFRVYRFGQTKPVYVYRFLAQGTMEDKIYDRQVTKQSLSFRVVDQQQVERHFTMNELTELYTFEPDLLDDPNSEKKKKRDTPMLPKDTILAELLQIHKEHIVGYHEHDSLLDHKEEEELTEEERKAAWAEYEAEKKGLTMRFNIPTGTNLPPVSFNSQTPYIPFNLGALSAMSNQQLEDLINQGREKVVEATNSVTAVRIQPLEDIISAVWKENMNLSEAQVQALALSRQASQELDVKRREAIYNDVLTKQQMLISCVQRILMNRRLQQQYNQQQQQQMTYQQATLGHLMMPKPPNLIMNPSNYQQIDMRGMYQPVAGGMQPPPLQRAPPPMRSKNPGPSQGKSM

SEQ ID NO: 4 - DNA, 11167 bp, Homo sapiens
ctcggcccaacaaaatggcggcggcagcggtgtcgctttgtttccgcggctcctgcggcggtggcagtggtagcggcctttgagctgtggggaggttccagcagcagctacagtgacgactaagactccagtgcatttctatcgtaaccgggcgcgggggagcgcagatcggcgcccagcaatcacagaagccgacaaggcgttcaagcgaaaacatgaccgctgagcccatgagtgaaagcaagttgaatacattggtgcagaagcttcatgacttccttgcacactcatcagaagaatctgaagaaacaagttctcctccacgacttgcaatgaatcaaaacacagataaaatcagtggttctggaagtaactctgatatgatggaaaacagcaaggaagagggaactagctcttcagaaaaatccaagtcttcaggatcgtcacgatcaaagaggaaaccttcaattgtaacaaagtatgtagaatcagatgatgaaaaacctttggatgatgaaactgtaaatgaagatgcgtctaatgaaaattcagaaaatgatattactatgcagagcttgccaaaaggtacagtgattgtacagccagagccagtgctgaatgaagacaaagatgattttaaagggcctgaatttagaagcagaagtaaaatgaaaactgaaaatctcaaaaaacgcggagaagatgggcttcatgggattgtgagctgcactgcttgtggacaacaggtcaatcattttcaaaaagattccatttatagacacccttcattgcaagttcttatttgtaagaattgctttaagtattacatgagtgatgatattagccgtgactcagatggaatggatgaacaatgtaggtggtgtgcggaaggtggaaacttgatttgttgtgacttttgccataatgctttctgcaagaaatgcattctacgcaaccttggtcgaaaggagttgtccacaataatggatgaaaacaaccaatggtattgctacatttgtcacccagagcctttgttggacttggtcactgcatgtaacagcgtatttgagaatttagaacagttgttgcagcaaaataagaagaagataaaagttgacagtgaaaagagtaataaagtatatgaacatacatccagattttctccaaagaagactagttcaaattgtaatggagaagaaaagaaattagatgattcctgttctggctctgtaacctactcttattccgcactaattgtgcccaaagagatgattaagaaggcaaaaaaactgattgagaccacagccaacatgaactccagttatgttaaatttttaaagcaggcaacagataattcagaaatcagttctgctacaaaattacgtcagcttaaggcttttaagtctgtgttggctgatattaagaaggctcatcttgcattggaagaagacttaaattccgagtttcgagcgatggatgctgtaaacaaagagaaaaataccaaagagcataaagtcatagatgctaagtttgaaacaaaagcacgaaaaggagaaaaaccttgtgctttggaaaagaaggatatttcaaagtcagaagctaaactttcaagaaaacaggtagatagtgagcacatgcatcagaatgttccaacagaggaacaaagaacaaataaaagtaccggtggtgaacataagaaatctgatagaaaagaagaacctcaatatgaacctgccaacacttctgaagatttagacatggatattgtgtctgttccttcctcagttccagaagacatttttgagaatcttgagactgctatggaagttcagagttcagttgatcatcaaggggatggcagcagtggaactgaacaagaagtggagagttcatctgtaaaattaaatatttcttcaaaagacaacagaggaggtattaaatcaaaaactacagctaaagtaacaaaagaattatatgttaaactcactcctgtttccctttctaattccccaattaaaggtgctgattgtcaggaagttccacaagataaagatggctataaaagttgtggtctgaaccccaagttagagaaatgtggacttggacaggaaaacagtgataatgagcatttggttgaaaatgaagtttcattacttttagaggaatctgatcttcgaagatccccacgtgtaaagactacacccttgaggcgaccgacagaaactaaccctgtaacatctaattcagatgaagaatgtaatgaaacagttaaggagaaacaaaaactatcagttccagtgagaaaaaaggataagcgtaattcttctgacagtgctatagataatcctaagcctaataaattgccaaaatctaagcaatcagagactgtggatcaaaattcagattctgatgaaatgctagcaatcctcaaagaggtgagcaggatgagtcacagttcttcttcagatactgatattaatgaaattcatacaaaccataagactttgtatgatttaaagactcaggcggggaaagatgataaaggaaaaaggaaacgaaaaagttctacatctggctcagattttgatactaaaaagggcaaatcagctaagagctctataatttctaaaaagaaacgacaaacccagtctgagtcttctaattatgactcagaattagaaaaagagataaagagcatgagtaaaattggtgctgccagaaccaccaaaaaaagaattccaaatacaaaagattttgactcttctgaagatgagaaacacagcaaaaaaggaatggataatcaagggcacaaaaatttgaagacctcacaagaaggatcatctgatgatgctgaaagaaaacaagagagagagactttctcttcagcagaaggcacagttgataaagacacgaccatcatggaattaagagatcgacttcctaagaagcagcaagcaagtgcttccactgatggtgtcgataagctttctgggaaagaggagagttttacttctttggaagttagaaaagttgctgaaactaaagaaaagagcaagcatctcaaaaccaaaacatgtaaaaaagtacaggatggcttatctgatattgcagagaaattcctaaagaaagaccagagcgatgaaacttctgaagatgataaaaagcagagcaaaaagggaactgaagaaaaaaagaaaccttcagactttaagaaaaaagtaattaaaatggaacaacagtatgaatcttcatctgatggcactgaaaagttacctgagcgagaagaaatttgtcattttcctaagggcataaaacaaattaagaatggaacaactgatggagaaaagaaaagtaaaaaaataagagataaaacttctaaaaagaaggatgaattatctgattatgctgagaagtcaacagggaaaggagatagttgtgactcttcagaggataaaaagagtaagaatggagcatatggtagagagaagaaaaggtgcaagttgcttggaaagagttcaaggaagagacaagattgttcatcatctgatactgagaaatattccatgaaagaagatggttgtaactcttctgataagagactgaaaagaatagaattgagggaaagaagaaatttaagttcaaagagaaatactaaggaaatacaaagtggctcatcatcatctgatgctgaggaaagttctgaagataataaaaagaagaagcaaagaacttcatctaaaaagaaggcagtcattgtcaaggagaaaaagagaaactccctaagaacaagcactaaaaggaagcaagctgacattacatcctcatcttcttctgatatagaagatgatgatcagaattctataggtgagggaagcagcgatgaacagaaaattaagcctgtgactgaaaatttagtgctgtcttcacatactggattttgccaatcttcaggagatgaagccttatctaaatcagtgcctgtcacagtggatgatgatgatgacgacaatgatcctgagaatagaattgccaagaagatgcttttagaagaaattaaagccaatctttcctctgatgaggatggatcttcagatgatgagccagaagaagggaaaaaaagaactggaaaacaaaatgaagaaaacccaggagatgaggaagcaaaaaatcaagtcaattctgaatcagattcagattctgaagaatctaagaagccaagatacagacataggcttttgcggcacaaattgactgtgagtgacggagaatctggagaagaaaaaaagacaaagcctaaagagcataaagaagtcaaaggcagaaacagaagaaaggtgagcagtgaagattcagaagattctgattttcaggaatcaggagttagtgaagaagttagtgaatccgaagatgaacagcggcccagaacaaggtctgcaaagaaagcagagttggaagaaaatcagcggagctataaacagaaaaagaaaaggcgacgtattaaggttcaagaagattcatccagtgaaaacaagagtaattctgaggaagaagaggaggaaaaagaagaggaggaggaagaggaggaggaggaggaagaggaggaggaagatgaaaatgatgattccaagtctcctggaaaaggcagaaagaaaattcggaagattcttaaagatgataaactgagaacagaaacacaaaatgctcttaaggaagaggaagagagacgaaaacgtattgctgagagggagcgtgagcgagaaaaattgagagaggtgatagaaattgaagatgcttcacccaccaagtgtccaataacaaccaagttggttttagatgaagatgaagaaaccaaagaacctttagtgcaggttcatagaaatatggttatcaaattgaaaccccatcaagtagatggtgttcagtttatgtgggattgctgctgtgagtctgtgaaaaaaacaaagaaatctccaggttcaggatgcattcttgcccactgtatgggccttggtaagactttacaggtggtaagttttcttcatacagttcttttgtgtgacaaactggatttcagcacggcgttagtggtttgtcctcttaatactgctttgaattggatgaatgaatttgagaagtggcaagagggattaaaagatgatgagaagcttgaggtttctgaattagcaactgtgaaacgtcctcaggagagaagctacatgctgcagaggtggcaagaagatggtggtgttatgatcataggctatgagatgtatagaaatcttgctcaaggaaggaatgtgaagagtcggaaacttaaagaaatatttaacaaagctttggttgatccaggccctgattttgttgtttgtgatgaaggccatattctaaaaaatgaagcatctgctgtttctaaagctatgaattctatacgatcaaggaggaggattattttaacaggaacaccacttcaaaataacctaattgagtatcattgtatggttaattttatcaaggaaaatttacttggatccattaaggagttcaggaatagatttataaatccaattcaaaatggtcagtgtgcagattctaccatggtagatgtcagagtgatgaaaaaacgtgctcacattctctatgagatgttagctggatgtgttcagaggaaagattatacagcattaacaaaattcttgcctccaaaacacgaatatgtgttagctgtgagaatgacttctattcagtgcaagctctatcagtactacttagatcacttaacaggtgtgggcaataatagtgaaggtggaagaggaaaggcaggtgcaaagcttttccaagattttcagatgttaagtagaatatggactcatccttggtgtttgcagctagactacattagcaaagaaaataagggttattttgatgaagacagtatggatgaatttatagcctcagattctgatgaaacctccatgagtttaagctccgatgattatacaaaaaagaagaaaaaagggaaaaaggggaaaaaagatagtagctcaagtggaagtggcagtgacaatgatgttgaagtgattaaggtctggaattcaagatctcggggaggtggtgaaggaaatgtggatgaaacaggaaacaatccttctgtttctttaaaactggaagaaagtaaagctacttcttcttctaatccaagcagcccagctccagactggtacaaagattttgttacagatgctgatgctgaggttttagagcattctgggaaaatggtacttctctttgaaattcttcgaatggcagaggaaattggggataaagtccttgttttcagccagtccctcatatctctggacttgattgaagattttcttgaattagctagtagggagaagacagaagataaagataaaccccttatttataaaggtgaggggaagtggcttcgaaacattgactattaccgtttagatggttccactactgcacagtcaaggaagaagtgggctgaagaatttaatgatgaaactaatgtgagaggacgattatttatcatttctactaaagcaggatctctaggaattaatctggtagctgctaatcgagtaattatattcgacgcttcttggaatccatcttatgacatccagagtatattcagagtttatcgctttggacaaactaagcctgtttatgtatataggttcttagctcagggaaccatggaagataagatttatgatcggcaagtaactaagcagtcactgtcttttcgagttgttgatcagcagcaggtggagcgtcattttactatgaatgagcttactgaactttatacttttgagccagacttattagatgaccctaattcagaaaagaagaagaagagggatactcccatgctgccaaaggataccatacttgcagagctccttcagatacataaagaacacattgtaggataccatgaacatgattctcttttggaccacaaagaagaagaagagttgactgaagaagaaagaaaagcagcttgggctgagtatgaagcagagaagaagggactgaccatgcgtttcaacataccaactgggaccaatttaccccctgtcagtttcaactctcaaactccttatattcctttcaatttgggagccctgtcagcaatgagtaatcaacagctggaggacctcattaatcaaggaagagaaaaagttgtagaagcaacaaacagtgtgacagcagtgaggattcaacctcttgaggatataatttcagctgtatggaaggagaacatgaatctctcagaggcccaagtacaggcgttagcattaagtagacaagccagccaggagcttgatgttaaacgaagagaagcaatctacaatgatgtattgacaaaacaacagatgttaatcagctgtgttcagcgaatacttatgaacagaaggctccagcagcagtacaatcagcagcaacagcaacaaatgacttatcaacaagcaacactgggtcacctcatgatgccaaagcccccaaatttgatcatgaatccttctaactaccagcagattgatatgagaggaatgtatcagccagtggctggtggtatgcagccaccaccattacagcgtgcaccacccccaatgagaagcaaaaatccaggaccttcccaagggaaatcaatgtgattttgcactaaaagcttaatggattgttaaaatcatagaaagatcttttatttttttaggaatcaatgacttaacagaactcaactgtataaatagtttggtccccttaaatgccaatcttccatattagttttactttttttttttttaaatagggcataccatttcttcctgacatttgtcagtgatgttgcctagaatcttcttacacacgctgagtacagaagatatttcaaattgttttcagtgaaaacaagtccttccataatagtaacaactccacagatttcctctctaaatttttatgcctgcttttagcaaccataaaattgtcataaaattaataaatttaggaaagaataaagatttatatattcattctttacatataaaaacacacagctgagttcttagagttgattcctcaagttatgaaatacttttgtacttaatccatttcttgattaaagtgattgaaatggttttaatgttcttttgactgaagtctgaaactgggctcctgctttattgtctctgtgactgaaagttagaaactgagggttatctttgacacagaattgtgtgcaatattcttaaatactactgctctaaaagttggagaagtcttgcagttatcttagcattgtataaacagccttaagtatagcctaagaagagaattcctttttcttctttagtccttctgccattttttattttcagttatatgtgctgaaataattactggtaaaatttcagggttgtggattatcttccacacatgaattttctctctcctggcacgaatataaagcacatctcttaactgcatggtgccagtgctaatgcttcatcctgttgctggcagtgggatgtggacttagaaaatcaagttctagcattttagtaggttaacactgaagttgtggttgttaggttcacaccctgttttataaacaacatcaaaatggcagaaccattgctgactttaggttcacatgaggaatgtacttttaacaattcccagtactatcagtattgtgaaataattcctctgaaagataagaatcactggcttctatgcgcttcttttctctcatcatcatgttcttttaccccagtttccttacatttttttaaattgtttcagagtttgttttttttttagtttagattgtgaggcaattattaaatcaaaattaattcatccaatacccctttactagaagttttactagaaaatgtattacattttattttttcttaatccagttctgcaaaaatgacctataaatttattcatgtacaattttggttacttgaattgttaaagaaaacattgtttttgactatgggagtcaactcaacatggcagaaccatttttgagatgatgatacaacaggtagtgaaacagcttaagaattccaaaaaaaaaaaaaaaaaaaaaaaaaagaaaactgggtttgggctttgctttaggtatcactggattagaatgagtttaacattagctaaaactgctttgagttgtttggatgattaagagattgccatttttatcttggaagaactagtggtaaaacatccaagagcactaggattgtgatacagaatttgtgaggtttggtggatccacgcccctctcccccactttcccatgatgaaatatcactaataaatcctgtatatttagatattatgctagccatgtaatcagatttatttaattgggtggggcaggtgtgtatttactttagaaaaaatgaaaaagacaagatttatgagaaatatttgaaggcagtacactctggccaactgttaccagttggtatttctacaagttcagaatattttaaacctgatttactagacctgggaattttcaacatggtctaattatttactcaaagacatagatgtgaaaattttaggcaaccttctaaatctttttcaccatggatgaaactataacttaaagaataatacttagaagggttaattggaaatcagagtttgaaataaaacttggaccactttgtatacactcttctcacttgacattttagctatataatatgtactttgagtataacatcaagctttaacaaatatttaaagacaaaaaaatcacgtcagtaaaatactaaaaggctcatttttatatttgttttagatgttttaaatagttgcaatggattaaaaatgatgatttaaaatgttgcttgtaatacagttttgcctgctaaattctccacattttgtaacctgttttatttctttgggtgtaaagcgtttttgcttagtattgtgatattgtatatgttttgtcccagttgtatagtaatgtttcagtccatcatccagctttggctgctgaaatcatacagctgtgaagacttgcctttgtttctgttagactgcttttcagttctgtattgagtatcttaagtactgtagaaaagatgtcacttcttcctttaaggctgttttgtaatatatataaggactggaattgtgtttttaaagaaaagcattcaagtatgacaatatactatctgtgttttcaccattcaaagtgctgtttagtagttgaaacttaaactatttaatgtcatttaataaagtgaccaaaatgtgttgtgctctttattgtattttcacagctttgaaaatctgtgcacatactgtttcatagaaaatgtatagcttttgttgtcctatataatggtggttcttttgcacatttagttatttaatattgagaggtcacgaagtttggttattgaatctgttatatactaaattctgtaaagggagatctctcatctcaaaaagaatttacataccaggaagtccatgtgtgtttgtgttagttttggatgtctttgtgtaatccagccccatttcctgtttcccaacagctgtaacactcattttaagtcaagcagggctaccaacccacacttgatagaaaagctgcttaccattcagaagcttccttattacctggcctccaaatgagctgaatattttgtagccttcccttagctatgttcattttccctccattatcataaaatcagatcgatatttatgtgccccaaacaaaactttaagagcagttacattctgtcccagtagcccttgtttcctttgagagtagcatgttgtgaggctatagagacttattctaccagtaaaacaggtcaatccttttacatgtttattatactaaaaattatgttcagggtatttactactttatttcaccagactcagtctcaagtgacttggctatctccaaatcagatctacccttagagaataaacatttttctaccgttattttttttcaagtctataatctgagccagtcccaaaggagtgatcaagtttcagaaatgctttcatcttcacaacattttatatatactattatatggggtgaataaagttttaaatccgaaatata

SEQ ID NO: 5 - protein, 740 aa, Homo sapiens
MATANSIIVLDDDDEDEAAAQPGPSHPLPNAASPGAEAPSSSEPHGARGSSSSGGKKCYKLENEKLFEEFLELCKMQTADHPEVVPFLYNRQQRAHSLFLASAEFCNILSRVLSRARSRPAKLYVYINELCTVLKAHSAKKKLNLAPAATTSNEPSGNNPPTHLSLDPTNAENTASQSPRTRGSRRQIQRLEQLLALYVAEIRRLQEKELDLSELDDPDSAYLQEARLKRKLIRLFGRLCELKDCSSLTGRVIEQRIPYRGTRYPEVNRRIERLINKPGPDTFPDYGDVLRAVEKAAARHSLGLPRQQLQLMAQDAFRDVGIRLQERRHLDLIYNFGCHLTDDYRPGVDPALSDPVLARRLRENRSLAMSRLDEVISKYAMLQDKSEEGERKKRRARLQGTSSHSADTPEASLDSGEGPSGMASQGCPSASRAETDDEDDEESDEEEEEEEEEEEEEATDSEEEEDLEQMQEGQEDDEEEDEEEEAAAGKDGDKSPMSSLQISNEKNLEPGKQISRSSGEQQNKGRIVSPSLLSEEPLAPSSIDAESNGEQPEELTLEEESPVSQLFELEIEALPLDTPSSVETDISSSRKQSEEPFTTVLENGAGMVSSTSFNGGVSPHNWGDSGPPCKKSRKEKKQTGSGPLGNSYVERQRSVHEKNGKKICTLPSPPSPLASLAPVADSSTRVDSPSHGLVTSSLCIPSPARLSQTPHSQPPRPGTCKTSVATQCDPEEIIVLSDSD

SEQ ID NO: 6 - DNA, 2563 bp, Homo sapiens
acgacaatatggcggcgcttagttgcatgaaggcggaaactctgtgacttccggtccgtagtggggcctgcggtgggagtgggaaggaaggcggagggaaccatgcgaggttctgagaattgcggcgagggtcgcctcgagagacggtttctgagcatcatcgtgctggatgatgatgacgaagatgaagcagctgctcagccagggccctcccacccactccccaatgcggcctcacctggggcagaagcccctagctcctctgagcctcatggggccagaggaagcagtagttcgggcggcaagaaatgctacaagctggagaatgagaagctgttcgaagagttccttgaactttgtaagatgcagacagcagaccaccctgaggtggtcccattcctctataaccggcagcaacgtgcccactctctgtttttggcctcggcggagttctgcaacatcctctctagggtcctgtctcgggcccggagccggccagccaagctctatgtctacatcaatgagctctgcactgttctcaaggcccactcagccaaaaagaagctgaacttggcccctgccgccaccacctccaatgagccctctgggaataaccctcccacacacctctccttggaccccacaaatgctgaaaacactgcctctcagtctccaaggacccgtggttcccggcggcagatccagcgtttggagcagctgctggcgctctatgtggcagagatccggcggctgcaggaaaaggagttggatctctcagaattggatgacccagactccgcatacctgcaggaggcacggttgaagcgtaagctgatccgcctctttgggcgactatgtgagctgaaagactgctcttcactgaccggccgtgtcatagagcagcgcatcccctaccgtggcacccgctacccagaggttaacaggcgcattgagcggctcatcaacaagccagggcctgataccttccctgactatggggatgtgcttcgggctgtagagaaggcagctgcccgacacagccttggcctcccccgacagcagctccagctcatggctcaggatgccttccgagatgtgggcatcaggttacaggagcgacgtcacctcgatctcatctacaactttggctgccacctcacagatgactataggccaggcgttgaccctgcactatcagatcctgtgttggcccggcgccttcgggaaaaccggagtttggccatgagtcggctggatgaggtcatctccaaatatgcaatgttgcaagacaaaagtgaggagggcgagagaaaaaagagaagagctcggctccaaggcacctcttcccactctgcagacacccccgaagcctccttggattctggtgagggccctagtggaatggcatcccaggggtgcccttctgcctccagagctgagacagatgacgaagacgatgaggagagtgatgaggaagaggaggaggaggaggaagaagaagaggaggaggccacagattctgaagaggaggaggatctggaacagatgcaggagggtcaggaggatgatgaagaggaggacgaagaggaagaagcagcagcaggtaaagatggagacaagagccccatgtcctcactacagatctccaatgaaaagaacctggaacctggcaaacagatcagcagatcttcaggggagcagcaaaacaaaggacgcatagtgtcaccatcgttactgtcagaagaacccctggccccctccagcatagatgctgaaagcaatggagaacagcctgaggagctgaccctggaggaagaaagccctgtgtctcagctctttgagctagagattgaagctttgcccctggataccccttcctctgtggagacggacatttcctcttccaggaagcaatcagaggagcccttcaccactgtcttagagaatggagcaggcatggtctcttctacttccttcaatggaggcgtctctcctcacaactggggagattctggtcccccctgcaaaaaatctcggaaggagaagaagcaaacaggatcagggccattaggaaacagctatgtggaaaggcaaaggtcagtgcatgagaagaatgggaaaaagatatgtaccctgcccagcccaccttcccccttggcttccttggccccagttgctgattcctccacgagggtggactctcccagccatggcctggtgaccagctccctctgcatcccttctccagcccggctgtcccaaaccccccattcacagcctcctcggcctggtacttgcaagacaagtgtggccacacaatgcgatccagaagagatcatcgtgctctcagactctgattagctgcctccccttctccctgcctccagaatgttctgggataacatttggaggaaggtgggaagcagatgactgaggaagggatggactaagctaatccccttttggtggtgtttctttaaaaaaaaaaaaaagcttaagttttacacagaaacattaataaacaataaagttcttttcttactgtaaaaaaaaaaaaaaaaaa

SEQ ID NO: 7 - DNA, 18 bp, Homo sapiens
tgttgattgggtacaatg

SEQ ID NO: 8 - DNA, 18 bp, Homo sapiens
gcaaaagcctccccttcg

SEQ ID NO: 9 - DNA, 18 bp, Homo sapiens
ggcatagctgctcgacag

SEQ ID NO: 10 - DNA, 20 bp, Homo sapiens
cgtagtcaaatggctctcac

SEQ ID NO: 11 - DNA, 20 bp, Homo sapiens
ccatttgactacgtggactg

SEQ ID NO: 12 - DNA, 20 bp, Homo sapiens
ccacagtccacgtagtcaaa

SEQ ID NO: 13 - DNA, 20 bp, Homo sapiens
tgactacgtggactgtggcc

SEQ ID NO: 14 - DNA, 20 bp, Homo sapiens
gtgagagccatttgactacg

SEQ ID NO: 15 - DNA, 20 bp, Homo sapiens
agagaatcgacaaaaggctc

SEQ ID NO: 16 - DNA, 19 bp, Homo sapiens
ttttccatgaatttacccg

SEQ ID NO: 17 - DNA, 19 bp, Homo sapiens
ttatgcaggaatgtcaacc

SEQ ID NO: 18 - DNA, 19 bp, Homo sapiens
attaccataattcttgctg

SEQ ID NO: 19 - DNA, 19 bp, Homo sapiens
atttgaagaaatgttcacc

SEQ ID NO: 20 - DNA, 19 bp, Homo sapiens
ttaaaaaacaatgtggccc

SEQ ID NO: 21 - DNA, 19 bp, Homo sapiens
aaatcaagaaagcttggtc

SEQ ID NO: 22 - DNA, 19 bp, Homo sapiens
aaatctagaatcttcactc

SEQ ID NO: 23 - DNA, 19 bp, Homo sapiens
attagaaaaatggtgcttc

SEQ ID NO: 24 - DNA, 19 bp, Homo sapiens
tttaagagaaggctagttc

SEQ ID NO: 25 - DNA, 19 bp, Homo sapiens
attacaagtactttagagc

SEQ ID NO: 26 - DNA, 19 bp, Homo sapiens
aatttgaagaccattaacc

SEQ ID NO: 27 - DNA, 19 bp, Homo sapiens
ttattcaataacctccaac

SEQ ID NO: 28 - DNA, 19 bp, Homo sapiens
atatggaagagaatgggcc

SEQ ID NO: 29 - DNA, 19 bp, Homo sapiens
attatcagtactgtcactg

SEQ ID NO: 30 - DNA, 19 bp, Homo sapiens
aaataaatgagaatggccg

SEQ ID NO: 31 - DNA, 19 bp, Homo sapiens
aattccattatgatgggag

SEQ ID NO: 32 - DNA, 19 bp, Homo sapiens
aaaaaaaagagactcttcc

SEQ ID NO: 33 - DNA, 19 bp, Homo sapiens
tatttgatgagacttacag

SEQ ID NO: 34 - DNA, 19 bp, Homo sapiens
ttatcaatgaatcttgctc

SEQ ID NO: 35 - DNA, 19 bp, Homo sapiens
attaccatcagtgtccacc

SEQ ID NO: 36 - DNA, 49 bp, Homo sapiens
gaatctcagtagctcatctaatcaagagttagatgagctactgagattc

SEQ ID NO: 37 - DNA, 49 bp, Homo sapiens
ggaagagcacagtaaactaattcaagagattagtttactgtgctcttcc

SEQ ID NO: 38 - DNA, 49 bp, Homo sapiens
gaccccaagctcgtgtctaattcaagagattagacacgagcttggggtc

SEQ ID NO: 39 - DNA, 49 bp, Homo sapiens
gcagaagatctacgacctatttcaagagaataggtcgtagatcttctgc

SEQ ID NO: 40 - DNA, 49 bp, Homo sapiens
gcagaacagcatcagaggacttcaagagagtcctctgatgctgttctgc

SEQ ID NO: 41 - DNA, 49 bp, Homo sapiens
gggagtcttgtaagccagtgatcaagagtcactggcttacaagactccc

SEQ ID NO: 42 - DNA, 49 bp, Homo sapiens
gccacagtccacgtagtcaaatcaagagtttgactacgtggactgtggc

SEQ ID NO: 43 - DNA, 49 bp, Homo sapiens
gctctcactggaatctctccttcaagagaggagagattccagtgagagc

SEQ ID NO: 44 - DNA, 49 bp, Homo sapiens
ggtccctaaacaacagctctttcaagagaagagctgttgtttagggacc

SEQ ID NO: 45 - DNA, 49 bp, Homo sapiens
ggaaatgaccaccaaggacaatcaagagttgtccttggtggtcatttcc

SEQ ID NO: 46 - DNA, 20 bp, Homo sapiens
ctgctcctctgtaagaggca

SEQ ID NO: 47 - DNA, 20 bp, Homo sapiens
ttcctctgctcctctgtaag

SEQ ID NO: 48 - DNA, 20 bp, Homo sapiens
tgcctcttacagaggagcag

SEQ ID NO: 49 - DNA, 20 bp, Homo sapiens
gattgaagagaatcgacaaa

SEQ ID NO: 50 - DNA, 20 bp, Homo sapiens
agagaatcgacaaaaggctc

SEQ ID NO: 51 - DNA, 20 bp, Homo sapiens
caataacttctcagctctgc

SEQ ID NO: 52 - DNA, 20 bp, Homo sapiens
ccaataacttctcagctctg

SEQ ID NO: 53 - DNA, 20 bp, Homo sapiens
ccgcagagctgagaagttat

SEQ ID NO: 54 - DNA, 20 bp, Homo sapiens
tattggcagaacagcatcag

SEQ ID NO: 55 - DNA, 20 bp, Homo sapiens
acagcatcagaggactagct

SEQ ID NO: 56 - DNA, 20 bp, Homo sapiens
cagcatcagaggactagctc

SEQ ID NO: 57 - DNA, 20 bp, Homo sapiens
gaatgggttgccagcaatgg

SEQ ID NO: 58 - DNA, 20 bp, Homo sapiens
agctcgggcacctccattgc

SEQ ID NO: 59 - DNA, 20 bp, Homo sapiens
ctggaatgggttgccagcaa

SEQ ID NO: 60 - DNA, 20 bp, Homo sapiens
ggccttgcttggcctggaat

SEQ ID NO: 61 - DNA, 20 bp, Homo sapiens
gggccttgcttggcctggaa

SEQ ID NO: 62 - DNA, 20 bp, Homo sapiens
gggatgggccttgcttggcc

SEQ ID NO: 63 - DNA, 20 bp, Homo sapiens
aacccattccaggccaagca

SEQ ID NO: 64 - DNA, 20 bp, Homo sapiens
gcccatcccaaaatttccca

SEQ ID NO: 65 - DNA, 20 bp, Homo sapiens
cccatcccaaaatttcccaa

SEQ ID NO: 66 - DNA, 20 bp, Homo sapiens
aaaatgacaccatggctcac

SEQ ID NO: 67 - DNA, 20 bp, Homo sapiens
tcttgtaagccagtgagcca

SEQ ID NO: 68 - DNA, 20 bp, Homo sapiens
gttgcttgaaaatgacacca

SEQ ID NO: 69 - DNA, 20 bp, Homo sapiens
tgtgggaatcatgaggtctt

SEQ ID NO: 70 - DNA, 20 bp, Homo sapiens
tgaaaactgtgggaatcatg

SEQ ID NO: 71 - DNA, 20 bp, Homo sapiens
tccctttgcctgaaaactgt

SEQ ID NO: 72 - DNA, 20 bp, Homo sapiens
ttccctttgcctgaaaactg

SEQ ID NO: 73 - DNA, 20 bp, Homo sapiens
caggctgtgggcatttcttc

SEQ ID NO: 74 - DNA, 20 bp, Homo sapiens
gaaatgcccacagcctgccc

SEQ ID NO: 75 - DNA, 20 bp, Homo sapiens
acgtggactgtggcctgggc

SEQ ID NO: 76 - DNA, 20 bp, Homo sapiens
gactacgtggactgtggcct

SEQ ID NO: 77 - DNA, 20 bp, Homo sapiens
tgactacgtggactgtggcc

SEQ ID NO: 78 - DNA, 20 bp, Homo sapiens
ccatttgactacgtggactg

SEQ ID NO: 79 - DNA, 20 bp, Homo sapiens
gtgagagccatttgactacg

SEQ ID NO: 80 - DNA, 20 bp, Homo sapiens
ccacagtccacgtagtcaaa

SEQ ID NO: 81 - DNA, 20 bp, Homo sapiens
cgtagtcaaatggctctcac

SEQ ID NO: 82 - DNA, 20 bp, Homo sapiens
cactggaatctctcctccct

SEQ ID NO: 83 - DNA, 20 bp, Homo sapiens
ggaggactttgtgccaaggg

SEQ ID NO: 84 - DNA, 20 bp, Homo sapiens
tctggaggactttgtgccaa

SEQ ID NO: 85 - DNA, 20 bp, Homo sapiens
ctctggaggactttgtgcca

SEQ ID NO: 86 - DNA, 20 bp, Homo sapiens
cttggcacaaagtcctccag

SEQ ID NO: 87 - DNA, 20 bp, Homo sapiens
tgttgtttagggacctctgg

SEQ ID NO: 88 - DNA, 20 bp, Homo sapiens
agctgttgtttagggacctc

SEQ ID NO: 89 - DNA, 20 bp, Homo sapiens
aactcaagagctgttgttta

SEQ ID NO: 90 - DNA, 20 bp, Homo sapiens
taactcaagagctgttgttt

SEQ ID NO: 91 - DNA, 20 bp, Homo sapiens
cagctcttgagttatgagtt

SEQ ID NO: 92 - DNA, 20 bp, Homo sapiens
ttgagttatgagttaggtca

SEQ ID NO: 93 - DNA, 20 bp, Homo sapiens
gttaggtcaaggtcatgctc

SEQ ID NO: 94 - DNA, 20 bp, Homo sapiens
aagggtgtgaacctgatctc

SEQ ID NO: 95 - DNA, 20 bp, Homo sapiens
ctcaggcttcacctgagatc

SEQ ID NO: 96 - DNA, 20 bp, Homo sapiens
ttatgagttgggttagcaaa

SEQ ID NO: 97 - DNA, 20 bp, Homo sapiens
cttatgagttgggttagcaa

SEQ ID NO: 98 - DNA, 20 bp, Homo sapiens
tggccagaggcttatgagtt

SEQ ID NO: 99 - DNA, 20 bp, Homo sapiens
ttggccagaggcttatgagt

SEQ ID NO: 100 - DNA, 20 bp, Homo sapiens
taacccaactcataagcctc

SEQ ID NO: 101 - DNA, 20 bp, Homo sapiens
gaactctttggtttggccag

SEQ ID NO: 102 - DNA, 20 bp, Homo sapiens
ctcttgggaactctttggtt

SEQ ID NO: 103 - DNA, 20 bp, Homo sapiens
ggtgtctcttgggaactctt

SEQ ID NO: 104 - DNA, 20 bp, Homo sapiens
ggaatgagctggtgtctctt

SEQ ID NO: 105 - DNA, 20 bp, Homo sapiens
aggaatgagctggtgtctct

SEQ ID NO: 106 - DNA, 20 bp, Homo sapiens
ggctgtccagaggaatgagc

SEQ ID NO: 107 - DNA, 20 bp, Homo sapiens
gagacaccagctcattcctc

SEQ ID NO: 108 - DNA, 20 bp, Homo sapiens
atccctgggaggctgtccag

SEQ ID NO: 109 - DNA, 20 bp, Homo sapiens
attcctctggacagcctccc

SEQ ID NO: 110 - DNA, 20 bp, Homo sapiens
ttcctctggacagcctccca

SEQ ID NO: 111 - DNA, 20 bp, Homo sapiens
tctaacttagcatccctggg

SEQ ID NO: 112 - DNA, 20 bp, Homo sapiens
gcctctaacttagcatccct

SEQ ID NO: 113 - DNA, 20 bp, Homo sapiens
ggcctctaacttagcatccc

SEQ ID NO: 114 - DNA, 20 bp, Homo sapiens
tcccagggatgctaagttag

SEQ ID NO: 115 - DNA, 20 bp, Homo sapiens
aatgttctgccccgaagggg

SEQ ID NO: 116 - DNA, 20 bp, Homo sapiens
gacagcaaaagcctcccctt

SEQ ID NO: 117 - DNA, 20 bp, Homo sapiens
acagcaaaagcctccccttc

SEQ ID NO: 118 - DNA, 20 bp, Homo sapiens
agaaatgttctgccccgaag

SEQ ID NO: 119 - DNA, 20 bp, Homo sapiens
aagaaatgttctgccccgaa

SEQ ID NO: 120 - DNA, 20 bp, Homo sapiens
taagaaatgttctgccccga

SEQ ID NO: 121 - DNA, 20 bp, Homo sapiens
ttacactctctgagctagaa

SEQ ID NO: 122 - DNA, 20 bp, Homo sapiens
gctcagagagtgtaacgccc

SEQ ID NO: 123 - DNA, 20 bp, Homo sapiens
agtgtaacgcccaggacaga

SEQ ID NO: 124 - DNA, 20 bp, Homo sapiens
ctgaggaccctgacttctgc

SEQ ID NO: 125 - DNA, 20 bp, Homo sapiens
tcagggtcctcagtccaaaa

SEQ ID NO: 126 - DNA, 20 bp, Homo sapiens
aaaaggagtgaactctcaga

SEQ ID NO: 127 - DNA, 20 bp, Homo sapiens
aaaggagtgaactctcagaa

SEQ ID NO: 128 - DNA, 20 bp, Homo sapiens
ctcagaagggaaagtgcgta

SEQ ID NO: 129 - DNA, 20 bp, Homo sapiens
aagggaaagtgcgtaaggaa

SEQ ID NO: 130 - DNA, 20 bp, Homo sapiens
aaggaacggcgatcgtttcc

SEQ ID NO: 131 - DNA, 20 bp, Homo sapiens
cattgtacccaatcaacacc

SEQ ID NO: 132 - DNA, 20 bp, Homo sapiens
gatcgtttccaggtgttgat

SEQ ID NO: 133 - DNA, 20 bp, Homo sapiens
atcgtttccaggtgttgatt

SEQ ID NO: 134 - DNA, 20 bp, Homo sapiens
ggtgttgattgggtacaatg

SEQ ID NO: 135 - DNA, 20 bp, Homo sapiens
aagttccacgtcttggtgtc

SEQ ID NO: 136 - DNA, 20 bp, Homo sapiens
catgctgaagttccacgtct

SEQ ID NO: 137 - DNA, 20 bp, Homo sapiens
gttgaccgtggggaggctct

SEQ ID NO: 138 - DNA, 20 bp, Homo sapiens
ggttgaccgtggggaggctc

SEQ ID NO: 139 - DNA, 20 bp, Homo sapiens
gctgcaggttgaccgtgggg

SEQ ID NO: 140 - DNA, 20 bp, Homo sapiens
gaggctgcaggttgaccgtg

SEQ ID NO: 141 - DNA, 20 bp, Homo sapiens
agaggctgcaggttgaccgt

SEQ ID NO: 142 - DNA, 20 bp, Homo sapiens
cagaggctgcaggttgaccg

SEQ ID NO: 143 - DNA, 20 bp, Homo sapiens
aggcccattccagaggctgc

SEQ ID NO: 144 - DNA, 20 bp, Homo sapiens
cacggtcaacctgcagcctc

SEQ ID NO: 145 - DNA, 20 bp, Homo sapiens
ctgccataggcccattccag

SEQ ID NO: 146 - DNA, 20 bp, Homo sapiens
tcaacctgcagcctctggaa

SEQ ID NO: 147 - DNA, 20 bp, Homo sapiens
caacctgcagcctctggaat

SEQ ID NO: 148 - DNA, 20 bp, Homo sapiens
cagcctctggaatgggccta

SEQ ID NO: 149 - DNA, 20 bp, Homo sapiens
gggtgactcgctgctgccat

SEQ ID NO: 150 - DNA, 20 bp, Homo sapiens
tgtccctcactgctggtgga

SEQ ID NO: 151 - DNA, 20 bp, Homo sapiens
ctgtccctcactgctggtgg

SEQ ID NO: 152 - DNA, 20 bp, Homo sapiens
ggcctgtccctcactgctgg

SEQ ID NO: 153 - DNA, 20 bp, Homo sapiens
gtcaccctccaccagcagtg

SEQ ID NO: 154 - DNA, 20 bp, Homo sapiens
tcaccctccaccagcagtga

SEQ ID NO: 155 - DNA, 20 bp, Homo sapiens
ctccaccagcagtgagggac

SEQ ID NO: 156 - DNA, 20 bp, Homo sapiens
accagcagtgagggacaggc

SEQ ID NO: 157 - DNA, 20 bp, Homo sapiens
ggatggagctgatggaaggc

SEQ ID NO: 158 - DNA, 20 bp, Homo sapiens
aaagggatggagctgatgga

SEQ ID NO: 159 - DNA, 20 bp, Homo sapiens
aatgaaagggatggagctga

SEQ ID NO: 160 - DNA, 20 bp, Homo sapiens
cctttgacaaatgaaaggga

SEQ ID NO: 161 - DNA, 20 bp, Homo sapiens
tcgccctttgacaaatgaaa

SEQ ID NO: 162 - DNA, 20 bp, Homo sapiens
atcgccctttgacaaatgaa

SEQ ID NO: 163 - DNA, 20 bp, Homo sapiens
ccatccctttcatttgtcaa

SEQ ID NO: 164 - DNA, 20 bp, Homo sapiens
catccctttcatttgtcaaa

SEQ ID NO: 165 - DNA, 20 bp, Homo sapiens
ggcgatgcatgctcatctcc

SEQ ID NO: 166 - DNA, 20 bp, Homo sapiens
gcgatgcatgctcatctcca

SEQ ID NO: 167 - DNA, 20 bp, Homo sapiens
gtctgcctcgaagtaggccc

SEQ ID NO: 168 - DNA, 20 bp, Homo sapiens
actgatgtctgcctcgaagt

SEQ ID NO: 169 - DNA, 20 bp, Homo sapiens
catctccagggcctacttcg

SEQ ID NO: 170 - DNA, 20 bp, Homo sapiens
ggcagacatcagttattcac

SEQ ID NO: 171 - DNA, 20 bp, Homo sapiens
tctgtttaaaaagcgcaata

SEQ ID NO: 172 - DNA, 20 bp, Homo sapiens
caagagaaagctccacttcc

SEQ ID NO: 173 - DNA, 20 bp, Homo sapiens
caggaagtggagctttctct

SEQ ID NO: 174 - DNA, 20 bp, Homo sapiens
ggtccagctgaacttgtggg

SEQ ID NO: 175 - DNA, 20 bp, Homo sapiens
gagggtccagctgaacttgt

SEQ ID NO: 176 - DNA, 20 bp, Homo sapiens
agagggtccagctgaacttg

SEQ ID NO: 177 - DNA, 20 bp, Homo sapiens
ctgcctcccacaagttcagc

SEQ ID NO: 178 - DNA, 20 bp, Homo sapiens
gggtgagagtcgtgggcaga

SEQ ID NO: 179 - DNA, 20 bp, Homo sapiens
agggtgagagtcgtgggcag

SEQ ID NO: 180 - DNA, 20 bp, Homo sapiens
aacgccagggtgagagtcgt

SEQ ID NO: 181 - DNA, 20 bp, Homo sapiens
aaacgccagggtgagagtcg

SEQ ID NO: 182 - DNA, 20 bp, Homo sapiens
tctgcccacgactctcaccc

SEQ ID NO: 183 - DNA, 20 bp, Homo sapiens
gagctgagaagcaaacgcca

SEQ ID NO: 184 - DNA, 20 bp, Homo sapiens
tgagctgagaagcaaacgcc

SEQ ID NO: 185 - DNA, 20 bp, Homo sapiens
aggtctgcctctgggacatc

SEQ ID NO: 186 - DNA, 20 bp, Homo sapiens
tctcacgccagatgtcccag

SEQ ID NO: 187 - DNA, 20 bp, Homo sapiens
cttcagaaaggtctgcctct

SEQ ID NO: 188 - DNA, 20 bp, Homo sapiens
acttcagaaaggtctgcctc

SEQ ID NO: 189 - DNA, 20 bp, Homo sapiens
gcttggggtccacttcagaa

SEQ ID NO: 190 - DNA, 20 bp, Homo sapiens
agaggcagacctttctgaag

SEQ ID NO: 191 - DNA, 20 bp, Homo sapiens
tcagattagacacgagcttg

SEQ ID NO: 192 - DNA, 20 bp, Homo sapiens
atcagattagacacgagctt

SEQ ID NO: 193 - DNA, 20 bp, Homo sapiens
catcagattagacacgagct

SEQ ID NO: 194 - DNA, 20 bp, Homo sapiens
ttgactccagctctctgaaa

SEQ ID NO: 195 - DNA, 20 bp, Homo sapiens
caggcggcctcctttggcta

SEQ ID NO: 196 - DNA, 20 bp, Homo sapiens
gagcagcaggcggcctcctt

SEQ ID NO: 197 - DNA, 20 bp, Homo sapiens
tgtcgtcagcgagcagcagg

SEQ ID NO: 198 - DNA, 20 bp, Homo sapiens
ccatgtcgtcagcgagcagc

SEQ ID NO: 199 - DNA, 20 bp, Homo sapiens
cctgctgctcgctgacgaca

SEQ ID NO: 200 - DNA, 20 bp, Homo sapiens
ctgctgctcgctgacgacat

SEQ ID NO: 201 - DNA, 20 bp, Homo sapiens
gctcgctgacgacatgggcc

SEQ ID NO: 202 - DNA, 20 bp, Homo sapiens
ctcgctgacgacatgggcct

SEQ ID NO: 203 - DNA, 20 bp, Homo sapiens
tcgctgacgacatgggcctg

SEQ ID NO: 204 - DNA, 20 bp, Homo sapiens
tgcgatgcagatggcttgga

SEQ ID NO: 205 - DNA, 20 bp, Homo sapiens
aggctgcgatgcagatggct

SEQ ID NO: 206 - DNA, 20 bp, Homo sapiens
gtaaaaggctgcgatgcaga

SEQ ID NO: 207 - DNA, 20 bp, Homo sapiens
cggccactccttccggtaaa

SEQ ID NO: 208 - DNA, 20 bp, Homo sapiens
gatggcaccaccaccaggag

SEQ ID NO: 209 - DNA, 20 bp, Homo sapiens
gaaggagtggccgctcctgg

SEQ ID NO: 210 - DNA, 20 bp, Homo sapiens
cggaggatggcaccaccacc

SEQ ID NO: 211 - DNA, 20 bp, Homo sapiens
caggtgaagcgcacggagga

SEQ ID NO: 212 - DNA, 20 bp, Homo sapiens
ctcccaggtgaagcgcacgg

SEQ ID NO: 213 - DNA, 20 bp, Homo sapiens
ctgctcccaggtgaagcgca

SEQ ID NO: 214 - DNA, 20 bp, Homo sapiens
catcctccgtgcgcttcacc

SEQ ID NO: 215 - DNA, 20 bp, Homo sapiens
atcctccgtgcgcttcacct

SEQ ID NO: 216 - DNA, 20 bp, Homo sapiens
cgtgcgcttcacctgggagc

SEQ ID NO: 217 - DNA, 20 bp, Homo sapiens
agatggcagccaccgaagga

SEQ ID NO: 218 - DNA, 20 bp, Homo sapiens
tcagagatggcagccaccga

SEQ ID NO: 219 - DNA, 20 bp, Homo sapiens
atgcaatctgggctcagaga

SEQ ID NO: 220 - DNA, 20 bp, Homo sapiens
ccacgacgttgatgcaatct

SEQ ID NO: 221 - DNA, 20 bp, Homo sapiens
accacgacgttgatgcaatc

SEQ ID NO: 222 - DNA, 20 bp, Homo sapiens
cccagattgcatcaacgtcg

SEQ ID NO: 223 - DNA, 20 bp, Homo sapiens
tgcatcaacgtcgtggtgac

SEQ ID NO: 224 - DNA, 20 bp, Homo sapiens
gcatcaacgtcgtggtgact

SEQ ID NO: 225 - DNA, 20 bp, Homo sapiens
caacgtcgtggtgactggga

SEQ ID NO: 226 - DNA, 20 bp, Homo sapiens
tgatcaggccagctgtcagg

SEQ ID NO: 227 - DNA, 20 bp, Homo sapiens
tgttgatcaggccagctgtc

SEQ ID NO: 228 - DNA, 20 bp, Homo sapiens
gggaaggaccgcctgacagc

SEQ ID NO: 229 - DNA, 20 bp, Homo sapiens
caaagctgacaatgttgatc

SEQ ID NO: 230 - DNA, 20 bp, Homo sapiens
ctttgaccttcttagcaagt

SEQ ID NO: 231 - DNA, 20 bp, Homo sapiens
aatgatgacaactttaaaag

SEQ ID NO: 232 - DNA, 20 bp, Homo sapiens
caatgatgacaactttaaaa

SEQ ID NO: 233 - DNA, 20 bp, Homo sapiens
ctcacttcctcaaaaacagt

SEQ ID NO: 234 - DNA, 20 bp, Homo sapiens
cggcatagctgctcgacagc

SEQ ID NO: 235 - DNA, 20 bp, Homo sapiens
ccggcatagctgctcgacag

SEQ ID NO: 236 - DNA, 20 bp, Homo sapiens
ccgctgtcgagcagctatgc

SEQ ID NO: 237 - DNA, 20 bp, Homo sapiens
agcagctatgccggtcctaa

SEQ ID NO: 238 - DNA, 20 bp, Homo sapiens
cgacaacaggatcaccctct

SEQ ID NO: 239 - DNA, 20 bp, Homo sapiens
tggctggtgtgcccgacaac

SEQ ID NO: 240 - DNA, 20 bp, Homo sapiens
aagagggtgatcctgttgtc

SEQ ID NO: 241 - DNA, 20 bp, Homo sapiens
atctgcgtgtagagctctgc

SEQ ID NO: 242 - DNA, 20 bp, Homo sapiens
gatctgcgtgtagagctctg

SEQ ID NO: 243 - DNA, 20 bp, Homo sapiens
tgaaactgggggaagaaagt

SEQ ID NO: 244 - DNA, 20 bp, Homo sapiens
gtccaaaggcatgaaactgg

SEQ ID NO: 245 - DNA, 20 bp, Homo sapiens
agtccaaaggcatgaaactg

SEQ ID NO: 246 - DNA, 20 bp, Homo sapiens
aagtccaaaggcatgaaact

SEQ ID NO: 247 - DNA, 20 bp, Homo sapiens
gaagtccaaaggcatgaaac

SEQ ID NO: 248 - DNA, 20 bp, Homo sapiens
ttcccccagtttcatgcctt

SEQ ID NO: 249 - DNA, 20 bp, Homo sapiens
atcacagtagcgaagtccaa

SEQ ID NO: 250 - DNA, 20 bp, Homo sapiens
ttcgctactgtgatgccaaa

SEQ ID NO: 251 - DNA, 20 bp, Homo sapiens
cctgagtagtcccaccccca

SEQ ID NO: 252 - DNA, 20 bp, Homo sapiens
ccttgggggtgggactactc

SEQ ID NO: 253 - DNA, 20 bp, Homo sapiens
cttcagctctcccaggttgg

SEQ ID NO: 254 - DNA, 20 bp, Homo sapiens
ctactcaggttcctccaacc

SEQ ID NO: 255 - DNA, 20 bp, Homo sapiens
tactcaggttcctccaacct

SEQ ID NO: 256 - DNA, 20 bp, Homo sapiens
gagcttcagctctcccaggt

SEQ ID NO: 257 - DNA, 20 bp, Homo sapiens
gcaggagcttcagctctccc

SEQ ID NO: 258 - DNA, 20 bp, Homo sapiens
gggagagctgaagctcctgc

SEQ ID NO: 259 - DNA, 20 bp, Homo sapiens
gcatgactgcttcctccagc

SEQ ID NO: 260 - DNA, 20 bp, Homo sapiens
tggaggaagcagtcatgctg

SEQ ID NO: 261 - DNA, 20 bp, Homo sapiens
gggaaaggacgtcggacttg

SEQ ID NO: 262 - DNA, 20 bp, Homo sapiens
aggcagctgggaaaggacgt

SEQ ID NO: 263 - DNA, 20 bp, Homo sapiens
gcttggcaggcagctgggaa

SEQ ID NO: 264 - DNA, 20 bp, Homo sapiens
tgcgctgcttggcaggcagc

SEQ ID NO: 265 - DNA, 20 bp, Homo sapiens
actatcttgcgctgcttggc

SEQ ID NO: 266 - DNA, 20 bp, Homo sapiens
caccactatcttgcgctgct

SEQ ID NO: 267 - DNA, 20 bp, Homo sapiens
tgccaagcagcgcaagatag

SEQ ID NO: 268 - DNA, 20 bp, Homo sapiens
aagatagtggtgattgcccc

SEQ ID NO: 269 - DNA, 20 bp, Homo sapiens
cctggcattgatccgtcctg

SEQ ID NO: 270 - DNA, 20 bp, Homo sapiens
tcctggcattgatccgtcct

SEQ ID NO: 271 - DNA, 20 bp, Homo sapiens
gtcctggcattgatccgtcc

SEQ ID NO: 272 - DNA, 20 bp, Homo sapiens
ccccaggacggatcaatgcc

SEQ ID NO: 273 - DNA, 20 bp, Homo sapiens
atccagggcagctctggtcc

SEQ ID NO: 274 - DNA, 20 bp, Homo sapiens
tgcagcatccagggcagctc

SEQ ID NO: 275 - DNA, 20 bp, Homo sapiens
tgccaggaccagagctgccc

SEQ ID NO: 276 - DNA, 20 bp, Homo sapiens
ttccttggctgcagcatcca

SEQ ID NO: 277 - DNA, 20 bp, Homo sapiens
tttccttggctgcagcatcc

SEQ ID NO: 278 - DNA, 20 bp, Homo sapiens
tgccctggatgctgcagcca

SEQ ID NO: 279 - DNA, 20 bp, Homo sapiens
gtccttggtggtcatttcct

SEQ ID NO: 280 - DNA, 20 bp, Homo sapiens
agccaaggaaatgaccacca

SEQ ID NO: 281 - DNA, 20 bp, Homo sapiens
tctgttgaagaagagaatga

SEQ ID NO: 282 - DNA, 20 bp, Homo sapiens
tgaatatatcttggacctac

SEQ ID NO: 283 - DNA, 20 bp, Homo sapiens
tctctcttccactttccagt

SEQ ID NO: 284 - DNA, 20 bp, Homo sapiens
atcttggacctactggaaag

SEQ ID NO: 285 - DNA, 20 bp, Homo sapiens
tttagtatttgcacaccata

SEQ ID NO: 286 - DNA, 20 bp, Homo sapiens
ttgcgtccaggaccacctta

SEQ ID NO: 287 - DNA, 20 bp, Homo sapiens
tgcacaccataaggtggtcc

SEQ ID NO: 288 - DNA, 20 bp, Homo sapiens
gctcttgcgtaattgcgtcc

SEQ ID NO: 289 - DNA, 20 bp, Homo sapiens
atgaggtggagccatcgatg

SEQ ID NO: 290 - DNA, 20 bp, Homo sapiens
gtgcagcacatccgcatcga

SEQ ID NO: 291 - DNA, 20 bp, Homo sapiens
ctcccgctcagctgatgagg

SEQ ID NO: 292 - DNA, 20 bp, Homo sapiens
gtcctcccgctcagctgatg

SEQ ID NO: 293 - DNA, 20 bp, Homo sapiens
gctccacctcatcagctgag

SEQ ID NO: 294 - DNA, 20 bp, Homo sapiens
ctccacctcatcagctgagc

SEQ ID NO: 295 - DNA, 20 bp, Homo sapiens
cacctcatcagctgagcggg

SEQ ID NO: 296 - DNA, 20 bp, Homo sapiens
acagttggaactgctggcac

SEQ ID NO: 297 - DNA, 20 bp, Homo sapiens
tctccgacagttggaactgc

SEQ ID NO: 298 - DNA, 20 bp, Homo sapiens
cagcatgcctctccgacagt

SEQ ID NO: 299 - DNA, 20 bp, Homo sapiens
agcagttccaactgtcggag

SEQ ID NO: 300 - DNA, 20 bp, Homo sapiens
actgtcggagaggcatgctg

SEQ ID NO: 301 - DNA, 20 bp, Homo sapiens
ggcagcggtgatggacagca

SEQ ID NO: 302 - DNA, 20 bp, Homo sapiens
gcccatattggcagcggtga

SEQ ID NO: 303 - DNA, 20 bp, Homo sapiens
ggtgaggcccatattggcag

SEQ ID NO: 304 - DNA, 20 bp, Homo sapiens
gtccatcaccgctgccaata

SEQ ID NO: 305 - DNA, 20 bp, Homo sapiens
tccatcaccgctgccaatat

SEQ ID NO: 306 - DNA, 20 bp, Homo sapiens
ggagaaggtgaggcccatat

SEQ ID NO: 307 - DNA, 20 bp, Homo sapiens
ggtcagccgaggagaaggtg

SEQ ID NO: 308 - DNA, 20 bp, Homo sapiens
caccaggtcagccgaggaga

SEQ ID NO: 309 - DNA, 20 bp, Homo sapiens
tatgggcctcaccttctcct

SEQ ID NO: 310 - DNA, 20 bp, Homo sapiens
aaacaccaccaggtcagccg

SEQ ID NO: 311 - DNA, 20 bp, Homo sapiens
caccttctcctcggctgacc

SEQ ID NO: 312 - DNA, 20 bp, Homo sapiens
cttctcctcggctgacctgg

SEQ ID NO: 313 - DNA, 20 bp, Homo sapiens
acagctcagcaaacaccacc

SEQ ID NO: 314 - DNA, 20 bp, Homo sapiens
tctgtccaatgcggtgcacg

SEQ ID NO: 315 - DNA, 20 bp, Homo sapiens
gaggaccgcgtgcaccgcat

SEQ ID NO: 316 - DNA, 20 bp, Homo sapiens
cggagctggtctgtccaatg

SEQ ID NO: 317 - DNA, 20 bp, Homo sapiens
gtagtgaatgcccacggagc

SEQ ID NO: 318 - DNA, 20 bp, Homo sapiens
cattggacagaccagctccg

SEQ ID NO: 319 - DNA, 20 bp, Homo sapiens
attggacagaccagctccgt

SEQ ID NO: 320 - DNA, 20 bp, Homo sapiens
cacgaggtagtgaatgccca

SEQ ID NO: 321 - DNA, 20 bp, Homo sapiens
cgtgggcattcactacctcg

SEQ ID NO: 322 - DNA, 20 bp, Homo sapiens
cagctgtgccctttgccacg

SEQ ID NO: 323 - DNA, 20 bp, Homo sapiens
cattcactacctcgtggcaa

SEQ ID NO: 324 - DNA, 20 bp, Homo sapiens
attcactacctcgtggcaaa

SEQ ID NO: 325 - DNA, 20 bp, Homo sapiens
gcacagctgatgactacctt

SEQ ID NO: 326 - DNA, 20 bp, Homo sapiens
ttaatcttctcttgaatcag

SEQ ID NO: 327 - DNA, 20 bp, Homo sapiens
tttaatcttctcttgaatca

SEQ ID NO: 328 - DNA, 20 bp, Homo sapiens
ctttaatcttctcttgaatc

SEQ ID NO: 329 - DNA, 20 bp, Homo sapiens
tcaagagaagattaaagttc

SEQ ID NO: 330 - DNA, 20 bp, Homo sapiens
attaaagttctggcagaagc

SEQ ID NO: 331 - DNA, 20 bp, Homo sapiens
ttaaagttctggcagaagcc

SEQ ID NO: 332 - DNA, 20 bp, Homo sapiens
ttctgtcatttctgaaaaat

SEQ ID NO: 333 - DNA, 20 bp, Homo sapiens
atccactgattacctctaca

SEQ ID NO: 334 - DNA, 20 bp, Homo sapiens
cgtagatcttctgctgcttt

SEQ ID NO: 335 - DNA, 20 bp, Homo sapiens
tcgtagatcttctgctgctt

SEQ ID NO: 336 - DNA, 20 bp, Homo sapiens
tctcaaaggacttctggaat

SEQ ID NO: 337 - DNA, 20 bp, Homo sapiens
cttctttctcaaaggacttc

SEQ ID NO: 338 - DNA, 20 bp, Homo sapiens
atcacttccttctttctcaa

SEQ ID NO: 339 - DNA, 20 bp, Homo sapiens
cagaagtcctttgagaaaga

SEQ ID NO: 340 - DNA, 20 bp, Homo sapiens
tgagaaagaaggaagtgata

SEQ ID NO: 341 - DNA, 20 bp, Homo sapiens
aggaagtgatatggagctcc

SEQ ID NO: 342 - DNA, 20 bp, Homo sapiens
caaaggactctgctgcttcc

SEQ ID NO: 343 - DNA, 20 bp, Homo sapiens
tgaagcacttcctgggtcaa

SEQ ID NO: 344 - DNA, 20 bp, Homo sapiens
gcagcagagtcctttgaccc

SEQ ID NO: 345 - DNA, 20 bp, Homo sapiens
atgttcctgaagcacttcct

SEQ ID NO: 346 - DNA, 20 bp, Homo sapiens
gatgttcctgaagcacttcc

SEQ ID NO: 347 - DNA, 20 bp, Homo sapiens
tttgacccaggaagtgcttc

SEQ ID NO: 348 - DNA, 20 bp, Homo sapiens
ggaagtgcttcaggaacatc

SEQ ID NO: 349 - DNA, 20 bp, Homo sapiens
cagggtgtctcccatgttct

SEQ ID NO: 350 - DNA, 20 bp, Homo sapiens
ccagggtgtctcccatgttc

SEQ ID NO: 351 - DNA, 20 bp, Homo sapiens
ggaagtagttcccagaacat

SEQ ID NO: 352 - DNA, 20 bp, Homo sapiens
ccagaacatgggagacaccc

SEQ ID NO: 353 - DNA, 20 bp, Homo sapiens
tgtcaatgagctttcatcca

SEQ ID NO: 354 - DNA, 20 bp, Homo sapiens
ctgtcaatgagctttcatcc

SEQ ID NO: 355 - DNA, 20 bp, Homo sapiens
cagccagtccacagaagaaa

SEQ ID NO: 356 - RNA, 25 nt, Homo sapiens
uuucacagagaaaugcuuaugcagg

SEQ ID NO: 357 - DNA, 21 bp, Homo sapiens
caacaagatgaagagcaccaa

SEQ ID NO: 358 - DNA, 21 bp, Homo sapiens
ccatcagctccatccctttca

SEQ ID NO: 359 - DNA, 21 bp, Homo sapiens
aggtgttgattgggtacaatg